(19) United States
(12) Patent Application Publication
(10) Pub. No.: US 2024/0254209 A1
(43) Pub. Date: Aug. 1, 2024

(54) GENERATION OF HUMAN PEANUT ALLERGEN-SPECIFIC IGE MONOCLONAL ANTIBODIES FOR DIAGNOSTIC AND THERAPEUTIC USE

(71) Applicant: VANDERBILT UNIVERSITY, Nashville, TN (US)

(72) Inventor: Scott A. SMITH, White Bluff, TN (US)

(21) Appl. No.: 18/549,844

(22) PCT Filed: Mar. 9, 2022
(86) PCT No.: PCT/US22/19503

§ 371 Date: Sep. 8, 2023

Related U.S. Application Data
(60) Provisional application No. 63/159,764, filed on Mar. 11, 2021.

(51) Int. Cl. C07K 16/16; A61K 39/00; A61K 39/35; A61P 37/08; G01N 33/68

(52) U.S. Cl. CPC C07K 16/16; A61K 39/35; A61P 37/08; G01N 33/6854; A61K 2039/505; C07K 2317/21; G01N 2333/415; G01N 2800/24

(57) ABSTRACT

The present disclosure is directed to human monoclonal IgE antibodies, and IgG antibodies engineered therefrom. Such engineered antibodies can be used to blunt pathologic IgE responses in subjects, such as in the detection, treatment and prevention of allergies, such as those to peanut allergens.

### PRIORITY CLAIM

This application claims benefit of priority to U.S. Provisional Application Ser. No. 63/159,764, filed Mar. 11, 2021, the entire contents of which are hereby incorporated by reference.

### FEDERAL FUNDING STATEMENT

This invention was made with government support under grant nos. R21AI123307 and R01AI155668 awarded by the National Institutes of Health. The government has certain rights in the invention.

### BACKGROUND

### 1. Field of the Disclosure

The present disclosure relates generally to the fields of medicine, allergies, and immunology. More particular, the disclosure relates to human IgE monoclonal antibodies binding to allergic targets such as peanut antigens.

### 2. Background

Peanut allergy is a type of food allergy to peanuts. It is different from tree nut allergies, with peanuts being legumes and not true nuts. Physical symptoms of allergic reaction can include itchiness, hives, swelling, eczema, sneezing, asthma attack, abdominal pain, drop in blood pressure, diarrhea, and cardiac arrest. Anaphylaxis may occur. Those with a history of asthma are more likely to be severely affected.

The allergy is recognized as one of the most severe food allergies due to its prevalence, persistency, and potential severity of allergic reaction. In the United States, peanut allergy is present in 0.6% of the population. Among children in the Western world, rates are between 1.5% and 3% and have increased over time. It is a common cause of food-related fatal and near-fatal allergic reactions.

The cause of peanut allergy is unclear and at least 11 peanut allergens have been described. The condition is associated with several specific proteins categorized according to four common food allergy superfamilies: Cupin (Ara h 1), Prolamin (Ara h 2, 6, 7, 9), Profilin (Ara h 5), and Bet v-1-related proteins (Ara h 8). Among these peanut allergens, Ara h 1, Ara h 2, Ara h 3 and Ara h 6 are considered to be major allergens which means that they trigger an immunological response in more than 50% of the allergic population. These peanut allergens mediate an immune response via release of Immunoglobulin E (IgE) antibody as part of the allergic reaction.

Prevention may be partly achieved through early introduction of peanuts to the diets of pregnant women and babies. It is recommended that babies at high risk be given peanut products in areas where medical care is available as early as 4 months of age. The principal treatment for anaphylaxis is the injection of epinephrine.

Another preventive approach is immunotherapy, which involves attempting to reduce allergic sensitivity by repeated exposure to small amounts of peanut products; however, there is some evidence that this approach increases rather than decreases the risk of serious allergies. Peanut allergen powder has been approved by the U.S. FDA, but the cost is extremely high. At a minimum, there is an urgent need for additional research into this area to identify both improved preventative and therapeutic options.

### SUMMARY

Thus, in accordance with the present disclosure, there is provided a method of detecting a IgE antibody with binding affinity/specificity for a peanut antigen in a subject comprising (a) providing a test antibody or fragment thereof antibody or antibody fragment characterized by clone paired heavy and light chain CDRs from Tables 3 and 4; (b) contacting the test antibody or fragment thereof with an antibody-containing sample from said subject in the presence of a peanut antigen; and (c) detecting IgE antibody with binding affinity for peanut antigen in said sample by measuring the reduction of binding to peanut antigen by the test antibody or fragment thereof as compared to the binding of the test antibody or fragment thereof in the absence of said sample.

The sample may be a body fluid, or may be blood, sputum, tears, saliva, mucous or serum, urine, exudate, transudate, tissue scrapings or feces. Detection may comprise ELISA, RIA or Western blot, and/or said detection may be quantitative. The method may further comprise performing steps (a) and (b) a second time and determining a change in antibody levels as compared to the first assay. The test antibody or fragment thereof may be encoded by heavy and light chain variable sequences as set forth in Table 1, may be encoded by heavy and light chain variable sequences having 70%, 80%, or 90% identity to heavy and light chain variable sequences as set forth in Table 1, or may be encoded by heavy and light chain variable sequences having 95% identity to heavy and light chain variable sequences as set forth in Table 1. The test antibody or fragment thereof may comprise heavy and light chain variable sequences as set forth in Table 2, may comprise heavy and light chain variable sequences having 70%, 80% or 90% identity to heavy and light chain variable sequences as set forth in Table 2, or may comprise heavy and light chain variable sequences having 95% identity to heavy and light chain variable sequences as set forth in Table 2. The test antibody or fragment thereof may be an IgE antibody or IgG antibody, and the antibody fragment is a recombinant scFv (single chain fragment variable) antibody, Fab fragment, F(ab′)₂ fragment, or Fv fragment.

In another embodiment, there is provided a method of detecting a peanut allergen or antigen in a sample comprising (a) providing a test antibody or fragment thereof antibody or antibody fragment characterized by clone paired heavy and light chain CDRs from Tables 3 and 4; (b) contacting the test antibody or fragment thereof with a sample suspect of containing a peanut allergen or antigen; and (c) detecting a peanut allergen or antigen in said sample by binding of the test antibody or fragment. The sample may be an environmental sample or a food stuff. Detection may comprise ELISA, RIA or Western blot, and may be quantitative.

The test antibody or fragment thereof may be encoded by heavy and light chain variable sequences as set forth in Table 1, may be encoded by heavy and light chain variable sequences having 70%, 80%, or 90% identity to heavy and light chain variable sequences as set forth in Table 1, or may be encoded by heavy and light chain variable sequences having 95% identity to heavy and light chain variable sequences as set forth in Table 1. The test antibody or fragment thereof may comprise heavy and light chain variable sequences as set forth in Table 2, may comprise heavy and light chain variable sequences having 70%, 80% or 90% identity to heavy and light chain variable sequences as set forth in Table 2, or may comprise heavy and light chain variable sequences having 95% identity to heavy and light chain variable sequences as set forth in Table 2. The test antibody or fragment thereof may be an IgE antibody or IgG antibody, and the antibody fragment is a recombinant scFv (single chain fragment variable) antibody, Fab fragment, F(ab′)₂ fragment, or Fv fragment.

In yet another embodiment, there is provided a method of preventing or treating a peanut-related allergic reaction in a subject comprising delivering to said subject an IgG antibody or antibody fragment, wherein said antibody or antibody fragment is characterized by clone paired heavy and light chain CDRs from Tables 3 and 4. The antibody or fragment thereof may be encoded by heavy and light chain variable sequences as set forth in Table 1, may be encoded by heavy and light chain variable sequences having 70%, 80%, or 90% identity to heavy and light chain variable sequences as set forth in Table 1, or may be encoded by heavy and light chain variable sequences having 95% identity to heavy and light chain variable sequences as set forth in Table 1. The antibody or fragment thereof may comprise heavy and light chain variable sequences as set forth in Table 2, may comprise heavy and light chain variable sequences having 70%, 80% or 90% identity to heavy and light chain variable sequences as set forth in Table 2, or may comprise heavy and light chain variable sequences having 95% identity to heavy and light chain variable sequences as set forth in Table 2. The antibody fragment may be a recombinant scFv (single chain fragment variable) antibody, Fab fragment, F(ab′)₂ fragment, or Fv fragment, a chimeric antibody or a bispecific antibody.

The method may further comprised treating said subject with an anti-inflammatory agent, such as one selected from the group consisting of a steroid, an anti-histamine, and anti-leukotriene. The anti-inflammatory agent may be administered chronically. Delivering may comprise antibody or antibody fragment administration, or may comprise genetic delivery with an RNA or DNA sequence or vector encoding the antibody or antibody fragment.

A further embodiment comprises a monoclonal antibody or antibody fragment comprises clone paired heavy and light chain CDRs from Tables 3 and 4. The antibody or fragment thereof may be encoded by heavy and light chain variable sequences as set forth in Table 1, may be encoded by heavy and light chain variable sequences having 70%, 80%, or 90% identity to heavy and light chain variable sequences as set forth in Table 1, or may be encoded by heavy and light chain variable sequences having 95% identity to heavy and light chain variable sequences as set forth in Table 1. The antibody or fragment thereof may comprise heavy and light chain variable sequences as set forth in Table 2, may comprise heavy and light chain variable sequences having 70%, 80% or 90% identity to heavy and light chain variable sequences as set forth in Table 2, or may comprise heavy and light chain variable sequences having 95% identity to heavy and light chain variable sequences as set forth in Table 2. The antibody fragment may be a recombinant scFv (single chain fragment variable) antibody, Fab fragment, F(ab′)₂ fragment, or Fv fragment, a chimeric antibody or a bispecific antibody. The antibody may be an IgE, or is an IgG comprising grafted IgE CDRs or variable regions. The antibody or antibody fragment may further comprise a cell penetrating peptide and/or is an intrabody.

An additional embodiment comprises a hybridoma or engineered cell encoding an antibody or antibody fragment wherein the antibody or antibody fragment is characterized by clone paired heavy and light chain CDRs from Tables 3 and 4. The antibody or fragment thereof may be encoded by heavy and light chain variable sequences as set forth in Table 1, may be encoded by heavy and light chain variable sequences having 70%, 80%, or 90% identity to heavy and light chain variable sequences as set forth in Table 1, or may be encoded by heavy and light chain variable sequences having 95% identity to heavy and light chain variable sequences as set forth in Table 1. The antibody or fragment thereof may comprise heavy and light chain variable sequences as set forth in Table 2, may comprise heavy and light chain variable sequences having 70%, 80% or 90% identity to heavy and light chain variable sequences as set forth in Table 2, or may comprise heavy and light chain variable sequences having 95% identity to heavy and light chain variable sequences as set forth in Table 2. The antibody fragment may be a recombinant scFv (single chain fragment variable) antibody, Fab fragment, F(ab′)₂ fragment, or Fv fragment. The antibody may be a chimeric antibody, a bispecific antibody, is an IgE, or is an IgG. The antibody or antibody fragment may further comprise a cell penetrating peptide and/or is an intrabody.

A yet further embodiment is a vaccine formulation comprising one or more IgG antibodies or antibody fragments characterized by clone paired heavy and light chain CDRs from Tables 3 and 4. The antibody or fragment thereof may be encoded by heavy and light chain variable sequences as set forth in Table 1, may be encoded by heavy and light chain variable sequences having 70%, 80%, or 90% identity to heavy and light chain variable sequences as set forth in Table 1, or may be encoded by heavy and light chain variable sequences having 95% identity to heavy and light chain variable sequences as set forth in Table 1. The antibody or fragment thereof may comprise heavy and light chain variable sequences as set forth in Table 2, may comprise heavy and light chain variable sequences having 70%, 80% or 90% identity to heavy and light chain variable sequences as set forth in Table 2, or may comprise heavy and light chain variable sequences having 95% identity to heavy and light chain variable sequences as set forth in Table 2. The antibody fragment may be a recombinant scFv (single chain fragment variable) antibody, Fab fragment, F(ab′)₂ fragment, or Fv fragment, a chimeric antibody or a bispecific antibody. At least one of said antibodies or antibody fragments may further comprise a cell penetrating peptide and/or is an intrabody.

In yet an additional embodiment, there is provided a method of de-sensitizing a subject to a peanut allergen comprising (a) administering to said subject a peanut allergen; and (b) administering to said subject an IgG antibody or antibody fragment characterized by clone paired heavy and light chain CDRs from Tables 3 and 4. The peanut allergen and the IgG antibody may be mixed together prior to administering, may be administered to said subject separately, and/or are administered to said subject multiple times. The subject may be a human or a non-human mammal. The peanut allergen may be administered with an adjuvant.

Another embodiment is a method of producing an IgG immune response to a peanut allergen comprising (a) identifying an IgE epitope in an allergen by mapping the binding of an IgE antibody binding site; (b) modifying one or more residues in said IgE antibody binding site to reduce or eliminate IgE antibody binding to said binding site, thereby producing a hypoallergenic allergen; (c) immunizing a subject with said hypoallergenic allergen to produce and IgG response to said hypoallergenic allergen, while producing a reduced or no IgE response as compared to the allergen of step (a). The IgE antibody binding to said binding site may be reduced by at least 50%, by at least 90%, or may be eliminated. The hypoallergenic allergen may be administered to said subject with an adjuvant and/or is administered multiple times.

Also provide is method of determining the antigenic integrity of a peanut antigen comprising (a) contacting a sample comprising said peanut antigen with a first antibody or antibody fragment having clone-paired heavy and light chain CDR sequences from Tables 3 and 4, respectively; and (b) determining antigenic integrity of said peanut antigen by detectable binding of said antibody or antibody fragment to said antigen. The sample may comprise recombinantly produced antigen or a vaccine formulation or vaccine production batch. Detection may comprise ELISA, RIA, western blot, a biosensor using surface plasmon resonance or biolayer interferometry, or flow cytometric staining. The first antibody or antibody fragment may be encoded by clone-paired variable sequences as set forth in Table 1, encoded by light and heavy chain variable sequences having 70%, 80%, or 90% identity to clone-paired variable sequences as set forth in Table 1, or encoded by light and heavy chain variable sequences having 95% identity to clone-paired sequences as set forth in Table 1. The first antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The first antibody fragment may be a recombinant ScFv (single chain fragment variable) antibody, Fab fragment, F(ab′)₂ fragment, or Fv fragment. The method may further comprises performing steps (a) and (b) a second time to determine the antigenic stability of the antigen over time.

The method may further comprise (c) contacting a sample comprising said antigen with an antibody or antibody fragment having clone-paired heavy and light chain CDR sequences from Tables 3 and 4, respectively; and (d) determining antigenic integrity of said antigen by detectable binding of said antibody or antibody fragment to said antigen. The second antibody or antibody fragment may be encoded by clone-paired variable sequences as set forth in Table 1, encoded by light and heavy chain variable sequences having 70%, 80%, or 90% identity to clone-paired variable sequences as set forth in Table 1, or encoded by light and heavy chain variable sequences having 95% identity to clone-paired sequences as set forth in Table 1. The second antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The second antibody fragment may be a recombinant ScFv (single chain fragment variable) antibody, Fab fragment, F(ab′)₂ fragment, or Fv fragment. The method may further comprise performing steps (c) and (d) a second time to determine the antigenic stability of the antigen over time.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-F: Purified natural human IgE and peanut target proteins. Purified human peanut-specific IgE mAb 5C5 (FIG. 1A) is covalently coupled to sepharose (FIG. 1B) and used to purify target protein (FIG. 1C) Ara h 2. Human IgE 5C5 binds identically to natural Ara h 2 (nAra h 2) (FIG. 1D) and recombinant E. coli expressed Ara h 2 (rAra h 2) (FIG. 1E). Purified recombinant Ara h 6 was used to produce quantitative dose response curves (and calculate EC₅₀ values) for IgE mAb binding (FIG. 1F).

FIG. 2: Antigenic site mapping of Ara h 2 by competition ELISA. Isotype-switched variant IgG mAb coated ELISA plates are used to capture Ara h 2 and IgE mAb dilution series added. IgE isotype-specific HRP labeled secondary antibody is used to detect binding by the IgE mAb. Competition was said to occur if area under the curve (AUC) of IgE antibody binding is reduced by >75% from that same IgE antibody binding directly to its allergen target protein. Competition was said to not be occurring if AUC is reduced by <25%.

FIG. 3: Antigenic site map of Ara h 2 and Ara h 6 allergen proteins. Unique specific (SP) and cross-reactive (CR) antigenic sites are shown for the major peanut proteins Ara h 2 and 6. Ara h 6 and Ara h 2 serum-blocking studies suggest that mapping is complete—all immunodominant antigenic sites are accounted for.

FIG. 4: Site locations on Ara h 2. The structure of Ara h 2 (PDB 3OB4, (Mueller et al., 2011) is shown with human IgE antibody binding sites CR-A and SP-B highlighted in red. The disordered loop that is not captured in the original structure is shown in black.

FIGS. 5A-C: Human FcεRI transgenic mouse passive systemic anaphylaxis. Mice were sensitized using purified human IgE mAbs specific to Ara h 2 (FIG. 5A), Ara h 6 (FIG. 5B), or Ara h 2 & 6 (FIG. 5C) three days prior to challenge with peanut extract. Median overall survival is shown for the functional pairings, those mAbs capable of binding non-overlapping epitopes.

FIG. 6: Sensitization with single human IgE mAb does not result in anaphylaxis. Mice were sensitized with 100 mg of a single human IgE mAb: 40C7 (Ara h 1-specific), 5C5 (Ara h 2 site A-specific), 13D9 (Ara h 2 site B-specific), or 3C3 (Ara h 3-specific). Drop in temperature is shown following 10% peanut extract (ALK-Abello) challenge.

FIG. 7: Oral challenge of sensitized mice. Purified human peanut specific IgE mAbs 5C5, 13D9, 8F3, and 1H9 were injected (100 μg total) three days prior to oral challenge via gastric lavage using 100 μl freshly prepared peanut butter or peanut powder (Jif).

FIGS. 8A-B: Immunocapture serum IgE blocking analysis. Ara h 6-specific blocking IgG mAbs against antigenic site A (1H9 and site B (8F3) are used to quantify inhibition of their representative IgE population in seven peanut allergic sera (FIG. 8A). Percent inhibition is shown for IgE blocked in peanut and the Ara h 6 component ImmunoCAP test. Vin diagram (FIG. 8B) depicts the results of all sera blocking studies performed, showing the percentage of IgE directed toward the immunodominant antigenic sites of Ara h 2 and 6.

FIG. 9: Isotype-switched variant IgG inhibit passive systemic anaphylaxis. Mice were sensitized with 100 mg total of 5C5, 11F10, and 20G11 (Ara h 2 sites CR-A, CR-B, CR-C); one group of six mice also received IgG blocking mAb 16A8 (Ara h 2 site CR-A), and one group of six mice also received IgG blocking mAbs 16A8 and 13D9 (Ara h 2 sites CR-A, CR-B). Drop in temperature is shown following 10% peanut extract (ALK-Abello) challenge. Without therapeutic blocking only one mouse survived challenge.

### DESCRIPTION OF ILLUSTRATIVE EMBODIMENTS

As noted above, there is an urgent need to both better understand the immunologic biologic basis for peanut allergy as well as to provide improved preventative and therapeutic options for health professionals. The inventor here provides new human IgE antibodies to peanut antigens and proposes their use for preventing and treating peanut allegoric reactions. These and other aspects of the disclosure are described in detail below.

### I. IgE ANTIBODIES

### A. Biology

Immunoglobulin E (IgE), first discovered in 1966, is a kind of antibody (or immunoglobulin (Ig) “isotype”) that has only been found in mammals. IgE is synthesised by plasma cells. Monomers of IgE consist of two heavy chains (ε chain) and two light chains, with the ε chain containing 4 Ig-like constant domains (Cε1-Cε4). IgE's main function is immunity to parasites such as helminths like Schistosoma mansoni, Trichinella spiralis, and Fasciola hepatica. IgE is utilized during immune defense against certain protozoan parasites such as Plasmodium falciparum.

IgE also has an essential role in type I hypersensitivity, which manifests in various allergic diseases, such as allergic asthma, most types of sinusitis, allergic rhinitis, food allergies, and specific types of chronic urticaria and atopic dermatitis. IgE also plays a pivotal role in responses to allergens, such as: anaphylactic drugs, bee stings, and antigen preparations used in desensitization immunotherapy.

Although IgE is typically the least abundant isotype—blood serum IgE levels in a normal (“non-atopic”) individual are only 0.05% of the Ig concentration, compared to 75% for the IgGs at 10 mg/ml, which are the isotypes responsible for most of the classical adaptive immune response—it is capable of triggering the most powerful inflammatory reactions.

IgE primes the IgE-mediated allergic response by binding to Fc receptors found on the surface of mast cells and basophils. Fc receptors are also found on eosinophils, monocytes, macrophages and platelets in humans. There are two types of Fcε receptors, FcεRI (type I Fcε receptor), the high-affinity IgE receptor, and FcεRII (type II Fcε receptor), also known as CD23, the low-affinity IgE receptor. IgE can upregulate the expression of both types of Fcε receptors. FcεRI is expressed on mast cells, basophils, and the antigen-presenting dendritic cells in both mice and humans. Binding of antigens to IgE already bound by the FcεRI on mast cells causes cross-linking of the bound IgE and the aggregation of the underlying FcεRI, leading to the degranulation and the release of mediators from the cells. Basophils, upon the cross-linking of their surface IgE by antigens, release type 2 cytokines like interleukin-4 (IL-4) and interleukin-13 (IL-13) and other inflammatory mediators. The low-affinity receptor (FcεRII) is always expressed on B cells; but IL-4 can induce its expression on the surfaces of macrophages, eosinophils, platelets, and some T cells.

There is much speculation into what physiological benefits IgE contributes, and, so far, circumstantial evidence in animal models and statistical population trends have hinted that IgE may be beneficial in fighting gut parasites such as Schistosoma mansoni, but this has not been conclusively proven in humans. Epidemiological research shows that IgE level is increased when infected by Schistosoma mansoni, Necator americanus, and nematodes in human. It is most likely beneficial in removal of hookworms from the lung.

Although it is not yet well understood, IgE may play an important role in the immune system's recognition of cancer, in which the stimulation of a strong cytotoxic response against cells displaying only small amounts of early cancer markers would be beneficial. If this were the case, anti-IgE treatments such as omalizumab (for allergies) might have some undesirable side effects. However, a recent study, which was performed based on pooled analysis using comprehensive data from 67 phase I to IV clinical trials of omalizumab in various indications, concluded that a causal relationship between omalizumab therapy and malignancy is unlikely.

Atopic individuals can have up to 10 times the normal level of IgE in their blood (as do sufferers of hyper-IgE syndrome). However, this may not be a requirement for symptoms to occur as has been seen in asthmatics with normal IgE levels in their blood—recent research has shown that IgE production can occur locally in the nasal mucosa.

IgE that can specifically recognize an “allergen” (typically this is a protein, such as a peanut allergen, grass or ragweed pollen, etc.) has a unique long-lived interaction with its high-affinity receptor FcεRI so that basophils and mast cells, capable of mediating inflammatory reactions, become “primed”, ready to release chemicals like histamine, leukotrienes, and certain interleukins. These chemicals cause many of the symptoms are associated with allergy, such as airway constriction in asthma, local inflammation in eczema, increased mucus secretion in allergic rhinitis, and increased vascular permeability, it is presumed, to allow other immune cells to gain access to tissues, but which can lead to a potentially fatal drop in blood pressure as in anaphylaxis.

IgE is known to be elevated in various autoimmune disorders such as lupus (SLE), rheumatoid arthritis (RA) and psoriasis, and is theorized to be of pathogenetic importance in RA and SLE by eliciting a hypersensitivity reaction.

Regulation of IgE levels through control of B cell differentiation to antibody-secreting plasma cells is thought to involve the “low-affinity” receptor FcεRII, or CD23. CD23 may also allow facilitated antigen presentation, an IgE-dependent mechanism whereby B cells expressing CD23 are able to present allergen to (and stimulate) specific T helper cells, causing the perpetuation of a Th2 response, one of the hallmarks of which is the production of more antibodies.

Diagnosis of allergy is most often done by reviewing a person's medical history and finds a positive result for the presence of allergen specific IgE when conducting a skin or blood test. Specific IgE testing is the proven test for allergy detection; evidence does not show that indiscriminate IgE testing or testing for immunoglobulin G (IgG) can support allergy diagnosis.

### B. IgE-Mediated Allergic Diseases

The allergic response itself offers no evident advantage and is instead understood to be a side effect of the primary function of the IgE class of antibodies: to prevent infection by helminth worms (such as hookworm and schistosomes). Through mechanisms that are yet to be elucidated, allergens appear to be innocuous antigens that inappropriately produce an IgE antibody response that is typically specific for helminths.

For more than 50 years, the prevalence of allergic diseases has risen steadily in the industrialized world (Food Allergy Among U.S. Children: Trends in Prevalence and Hospitalizations. In the US, allergy is the fifth leading chronic disease in people of all ages and the third most common chronic disease in children (Sicherer et al., 1999 and American Academy of Allergy Asthma and Immunology: Food Allergy). IgE-mediated allergic diseases include asthma, atopic dermatitis, allergic rhinitis, allergic conjunctivitis, anaphylaxis, drug allergies, insect venom allergies, etc. These diseases are invoked and perpetuated by proteins contained in an array of plant and animal species that humans are exposed to on a daily basis. These allergen proteins exist in things like foods, venoms, drugs, trees, molds, mites, cockroaches, dogs, cats, latex, etc. Although allergy is among the country's most common diseases, it is often overlooked. New diagnostics and therapeutics are needed. Gaining a basic understanding of the molecular interactions at the heart of the pathogenesis of allergic diseases will open up new strategies for developing allergy diagnostics and therapeutics.

Asthma affects nearly 300 million individuals worldwide, about 25 million people in the U.S. alone. It affects all age groups, but it is children that are at the highest risk, with a prevalence that is rapidly growing. Asthma is the most prevalent cause of childhood disability in the U.S. and affects the poor disproportionately. Despite the prevalence, significant morbidity, and cost of this disease, little progress has been made with regard to understanding the pathogenesis or development of new strategies for treatment or prevention. Many of the allergens responsible for asthma are also associated with allergic rhinitis, affects between 10 and 30 percent of the population in developed countries. The most common indoor/outdoor triggers are dust mites, cockroaches, and cat, dog and rodent dander. Also of great importance, particularly in the case of allergic rhinitis, are trees, grasses, weed pollens, and mold spores.

Skin allergies are also very common and are one of the most important groups of allergic diseases that include eczema, hives, chronic hives and contact allergies. In the U.S., 8.8 million children have skin allergies, affecting the very young (age 0-4) disproportionately. Primary allergen culprits again include contact with dust mites and cockroaches, foods or even latex.

The most recent estimates suggest that up to 15 million Americans have allergies to food, and this number is rapidly rising. The Centers for Disease Control and Prevention reported that food allergies among children increased about 50% between 1997 and 2011, but there is no clear answer as to why (Food Allergy Among U.S. Children: Trends in Prevalence and Hospitalizations). The Centers for Disease Control also reported that food allergies result in more than 300,000 ambulatory-care and more than 200,000 emergency department visits a year among children (Sicherer et al., 1999). The economic cost of food allergies in children has reached nearly $25 billion per year. Food allergy is the leading cause of anaphylaxis outside the hospital setting. Eight foods account for 90 percent of all reactions: milk, eggs, peanuts, tree nuts, soy, wheat, fish and shellfish. Peanut and tree nut allergies, which tend to develop in childhood, are usually life-long, whereas cow's milk, egg and soy allergies are eventually outgrown. Approximately 3 million people report allergies to peanuts and tree nuts (Sicherer et al., 1999). The number of children living with peanut allergy has tripled between 1997 and 2008. There is no cure for food allergies. Strict avoidance of food allergens and early recognition and management of allergic reactions is the current strategy applied in clinical practices around the world. Unfortunately, even trace amounts of a food allergen can cause a reaction.

Despite the fact that IgE causes so much human suffering in the form of allergic disease, it was not until 1967 before the “reagin” molecule was discovered (Johansson and Bennich, 1967). This is due to its very low serum concentration relative to other antibody isotypes—over one hundred thousand-fold less than IgG in healthy individuals. Only one IgE secreting cell line (U266), or its derivatives (SKO-007), has been available to study—the atypical multiple myeloma described in the original paper (Johansson and Bennich, 1967 and Olsson and Kaplan, 1980). This IgE molecule has been of integral importance, used in thousands of studies as a reagent or for the generation of reagents. However, its target has never been identified, thus forcing investigators who wish to study the naturally occurring IgE antibody response to use polyclonal serum.

### II. MONOCLONAL ANTIBODIES AND PRODUCTION THEREOF

### A. General Methods

It will be understood that IgE monoclonal antibodies will have several applications. These include the production of diagnostic kits for use in detecting peanut allergens, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

In the case of human antibodies against natural pathogens, a suitable approach is to identify subjects that have been exposed to the pathogens, such as those who have been diagnosed as having contracted the disease, or those who have been vaccinated to generate protective immunity against the pathogen. Circulating anti-pathogen antibodies can be detected, and antibody producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984).

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. Here, antibodies with specificity for peanut antigens are provided.

In another aspect, there are provided monoclonal antibodies having clone-paired CDR's from the heavy and light chains as illustrated in Tables 3 and 4, respectively. Such antibodies may be produced by the clones discussed below in the Examples section using methods described herein. These antibodies bind to peanut antigens that are discussed above.

In yet another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 1 and the amino acid sequences of Table 2.

### C. Engineering of Antibody Sequences

In various embodiments, one may choose to engineer sequences of the identified antibodies for a variety of reasons, such as improved expression, improved cross-reactivity or diminished off-target binding. A particularly useful engineering of the disclosed IgE antibodies will be those converted into IgG's. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies were generated by subcloning heavy and light chain Fv DNAs from the cloning vector into an IgG plasmid vector, transfected into 293 Freestyle cells or CHO cells, and antibodies were collected an purified from the 293 or CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency. Modifications in the Fc region can be introduced to extend the in vivo half-life of the antibody, or to alter Fc mediated functions such as complement activation, antibody dependent cellular cytotoxicity (ADCC), and FcR-mediated phagocytosis.

Of central importance to the present disclosure is isotype modification involving changing a naturally occurring human IgE isotype variable sequence to an IgG isotype. By making this unnatural modification of the IgE antibody, a pathogenic molecule can be made to possess therapeutic functions. Aside from the theoretical benefit that IgE isotype antibodies may have in control of helminth infections, IgE antibodies are necessary for causing IgE-mediated allergy. The function of an IgE antibody is conveyed through its Fc region, which directs binding of the antibody to specific Fc receptors on various cells. By changing a natural human IgE to an IgG, one completely alters the Fc receptors that can be engaged—this has never been shown to occur naturally in humans since the IgG isotypes are deleted from the B cell DNA when it class-switched to IgE. It is the IgE antibody's ability to bind the Fc receptors, FcεRI and FcεRII, which endow its pathogenic function. By engineering a human IgE variable sequence into an IgG antibody isotype, the pathogenic molecule can no longer perform its harmful functions. Additionally, the engineered IgG antibody can then provide new, therapeutic functions through engagement with various Fcγ receptors, such as those found on the mast cell, including FcγRIIB. IgG antibodies that bind FcγRIIB on the surface of mast cells result in inhibitory signaling and inhibition of mediator release. For example, an allergen specific IgE antibody bound to FcεRI on mast cells will signal the release of inflammatory mediators upon binding its specific allergen—resulting in the diseases associated with allergy. However, an IgG made from the allergen specific IgE variable sequences would bind FcγRIIB on mast cells and inhibit mediator release upon binding the specific disease inciting allergen.

Other types of modifications include residue modification designed to reduce oxidation, aggregation, deamidation, and immunogenicity in humans. Other changes can lead to an increase in manufacturability or yield, or reduced tissue cross-reactivity in humans.

Modified antibodies may be made by any technique known to those of skill in the art, including expression through standard molecular biological techniques, or the chemical synthesis of polypeptides. Methods for recombinant expression are addressed elsewhere in this document.

### D. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a stepwise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage diplay and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

### F. Purification

In certain embodiments, the antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### G. IgE Antibody Generation Protocols

The following are a series of exemplary protocols for use in practicing the disclosed methods and producing the disclosed compositions.

### (i) Hybridoma Process Outline

1. Growth and maintenance of rh-IL-21, CD40L, BAFF-NIH3T3 cells (NIH3T3)
2. Growth and maintenance of HMMAs (HMMA2.5)
3. Isolation of subject PBMCs from blood
4. NIH3T3 activation of B-cells from subject PBMCs (96-well-plates)
5. ELISA screening of NIH3T3 activated B-cell cultures (384-well format)
6. HMMA ctyofusion and plating in growth medium (cells in 384-well-plates)
7. HAT selection medium is added
8. ELISA screening of hybridomas (384-well format)
9. Limiting dilution/enrichment dilution and flow cytometric sorting (384-well format)
10. ELISA screening of limiting dilution products
11. Transfer IgE positive hybridomas to a 48-well plate
12. Freeze back an aliquot then do an ELISA on 48-well plates (96-well format)
13. Transfer IgE positive hybridomas to a 12-well plate
14. Transfer IgE positive hybridomas to a T-75 flask
15. Grow final clonal hybridoma in 1 L SFM in 4×T-225 flasks
16. Grow the residual hybridoma cells in T-75 flask for RNA production (freeze back three aliquot pellets)
17. Harvest SFM and purify mAb by chromatography
   (ii) Polyclonal Activation of Human B cells with rh-IL-21, CD40L, BAFF-NIH3T3 Feeder Cells

### Materials

1. Subject sample
   a. PBMCs: 1×10⁻⁶ cells per plate
   b. Subject Tonsils/Adenoids: 1×10⁻⁶ cells per plate
2. Medium A (Stemcell Technologies, 03801)
3. Trypan blue (Gibco 15250-061)
4. CpG
   a. Order the oligonucleotide ZOEZOEZZZZZOEEZOEZZZT (SEQ ID NO: 219) from invitrogen at the 10 μmole scale (desalted)
   b. Dissolve in nuclease free water at a concentration of 2.5 mg/ml
   c. Aliquot and store at −20° C.
5. Irradiated rh-IL-21, CD40L, BAFF-NIH3T3 cell line
   a. rh-IL-21, CD40L, BAFF-NIH3T3 cells grown in Medium A are trypsinized, washed, and resuspened in Medium A
   b. Irradiate cells for 15-20 minutes using Cesium 137 irradiator
6. Filtered conditioned media from rh-IL-21, CD40L, BAFF-NIH3T3 cell line (containing rh-IL-21 and BAFF)
   a. Harvest supernatant of nearly confluent rh-IL-21, CD40L, BAFF-NIH3T3 cells grown in Medium A.
   b. Centrifuge supernatant at 2500 RPM to pellet cellular debris.
   c. Sterile filter supernatant through 0.22 μm filter and store at 4° C.
7. Goat anti-human Kappa unlabeled antibody (Southern Biotech; 1 mg/ml; Cat No: 2060-01)
8. Goat anti-human Lambda unlabeled antibody (Southern Biotech; 1 mg/ml; Cat No:2070-01)
9. rh-IL-21, CD40L, BAFF-NIH3T3 growth media (prepares en+ough for one 96 well plate at 300 μl/well)
   a. Add cells to solution containing the following components:
      i. 20 ml of Medium A
      ii. 12 ml of rh-IL-21, CD40L, BAFF-NIH3T3 conditioned media
      iii. 20 μl CpG stock
      iv. 1 μl of Goat anti-human Kappa unlabeled antibody (1 mg/ml)
      v. 1 μl of Goat anti-human Lambda unlabeled antibody (1 mg/ml)
      vi. 5×10⁵ irradiated rh-IL-21, CD40L, BAFF-NIH3T3 cells
         1. Add 250 μl of Pen/Strep/Glutamine (100×) and 250 μl of Amphotericin B (250 μg/ml) per plate of Tonsil/Adenoids
10. 96-well plates (Corning: 3997)
11. Matrix electronic Pipette 850 μl (Thermo Scientific 2014)
12. Matrix tips (Thermo Scientific 8042)
13. 500 ml Rapid Flow filter unit, 0.22 μm (Fisher 09-741-05)
14. Hyclone Pen/Strep/Glutamine solution (Thermo SV30082.01)
15. Amphotericin B; 250 μg/ml solution (Fisher MT-30-003-CF)

### Protocol

1. When using a frozen stock of Subject PBMCs or Tonsils/Adenoids (TAs), thaw samples rapidly in 37° C. water bath. Remove stock from the water bath as soon as it has thawed. When using freshly isolated PBMCs or TAs, skip steps 1-3.
2. Drop wise, add 1 ml of warmed Medium A to the cells
3. Resuspend the cells in 10 ml warmed Medium A
4. Centrifuge the cell suspension at 1,100 RPM for 5 min
5. Discard the supernatant and resuspend cells in 1 ml warmed Medium A
6. Count cells and assess viability with trypan blue staining
7. Add the cells to rh-IL-21, CD40L, BAFF-NIH3T3 growth media and plate them out into a 96-well plate. One plate for every 1 million viable PBMCs. Using an electronic multichannel pipette, dispense 300 μl/well of mixture containing PBMCs/TAs into a 96-well plate
8. Incubate plates at 37° C. with 5% CO₂ for 7-8 days
   a. Monitor cells closely as different cells grow at different rates
   b. Fresh TAs grow much more readily than frozen PBMCs or PBMCs from Red Cross filters
9. Screen plates by ELISA (see the Standard Human IgE Fluorescent ELISA protocol) after 7-8 days of incubation; check plates daily for growth of B cells.
10. Wells that are determined by ELISA to be producing desired IgE antibodies then are used for electrical cytofusion with HMMA cells (see B-cell/HMMA fusion protocol).
   (iii) Growth and Maintenance of HMMA 2.5 Cells

### Materials

1. HMMA 2.5 cells
2. 50 ml conical tubes (Falcon 352070)
3. Medium A (Stemcell Technologies, 03801)
4. Canted-neck tissue culture flasks (Falcon)
   a. T-25 (Falcon 353109)
   b. T-75 (Falcon 353136)
   c. T-150 (Falcon 355001)
   d. T-225 (Falcon 353139)
5. Cell scraper (Falcon 353087) or (Techno Plastic Products 99003)

### Protocol

1. If starting with a frozen stock of HMMA cells, thaw an aliquot of the cells rapidly at 37° C. Remove the stock from the water bath as soon as it has thawed
2. Gently transfer the cells to a 50 ml conical tube
3. Drop wise, add 1 ml of warmed Medium A to the cells
4. Resuspend the cells in 10 ml of warmed Medium A
5. Centrifuge the cells for 5 minutes at 1100 RPM in a swinging bucket centrifuge
6. Discard the supernatant
7. Resuspend the cells in 25-30 ml of warmed Medium A and transfer to a T-75 flask
8. Incubate at 37° C. with 5% CO₂
9. Split cells just before they become confluent and/or the medium starts to turn yellow
   a. Aspirate off the old media
   b. Add back fresh, warm Medium A
   c. Scrape the cells off the bottom of the flask
   d. Transfer the cells to a bigger flask, or split them amongst flasks of the same size
10. Split cells 3-5 days prior to performing fusions.
11. Cells should be about 80-90% confluent, and as close to 100% viable as possible, prior to harvesting for use in electrofusion. Do not replace culture medium less than 12 hours prior to fusion

### (iv) B-cell/HMMA Fusion

### Materials

1. BTX cytofusion media [gram amounts are for 500 ml of cytofusion media]
   a. 300 mM Sorbitol (Fisher, #BP439-500) [27.3 g]
   b. 0.1 mM Calcium Acetate (Fisher, #AC21105-2500) [0.008 g or 8 mg]
   c. 0.5 mM Magnesium Acetate (Fisher, #AC42387-0050) [0.0536 g or 53.6 mg]
   d. 1.0 mg/ml BSA (Sigma, #A2153) [0.5 g]
   e. Filter sterilize and store at 4° C.
2. BTX cytofusion cuvettes (BTX620: 2 mm gap width; 400 μl)
3. Cytofusion device:
   a. BTX ECM 2001
   b. BTX cuvette holder (BTX Safety Stand, Model 630B)
4. 384-well cell culture plates (Nunc, #164688)
5. 50×HAT (Sigma, #H0262)
6. Medium A (Stemcell Technologies, #03801)
7. Medium E (Stemcell Technologies, #03805)
8. HAT media
   a. 400 ml Medium A
   b. 100 ml Medium E
   c. One vial 50×HAT
9. Matrix electronic Pipette 850 μl (Thermo Scientific 2014)
10. Matrix tips (Thermo Scientific 8042)
11. Histopaque-1077 (Sigma-Aldrich; REF: 10771-6X100ML)

### Protocol

1. Perform Histopaque-1077 gradient on HMMAs as described in Isolation of Peripheral blood mononuclear cells from human blood protocol.
2. Count HMMA cells and resuspend them in warmed BTX cytofusion media at 5 million cells/ml. You will need 120 μl of 5×10⁶ cells/ml for each fusion; transfer them to a 1.5 ml microcentrifuge tube that contains 1 ml of warmed BTX cytofusion media; you may need several tubes depending on the desired number of fusions.
3. Gently resuspend the contents of an IgE positive B cell culture well (as determined by ELISA, see the Standard Human IgE Fluorescent ELISA protocol) and transfer them to a 1.5 ml microcentrifuge tube that contains 1 ml of warmed BTX cytofusion media.
4. Centrifuge the microcentrifuge tubes containing the HMMA cells and the microcentrifuge tubes containing the IgE positive B-cells (they remain in separate tubes at this point) at 3,000 RPM for 3 min in a tabletop centrifuge
5. Decant the supernatant
6. Resuspend the cell pellets in 1 ml of warmed BTX cytofusion media
7. Repeat the centrifugation, disposal of the supernatant, and resuspension of the pellet in cytofusion media two times (resulting in a total of 3 centrifugations). After the last centrifugation, DO NOT resuspend the pellot. Simply decant the supernatant and wait until step 9 to resuspend the cells
8. Resuspend the HMMA cell pellet in 1 ml of BTX cytofusion media (so that the concentration remains at 5 million cells/ml)
9. Use 120 μl of the HMMA cell solution at 5 million cells/ml to resuspend the positive B-cells in each microcentrifuge tube prior to transfer to a cytofusion cuvette
10. Transfer the mixture of HMMA and B-cells (volume approximately 200-250 μl) to a cytofusion cuvette
11. Place the cuvette(s) (device holds one or two cuvettes) into the cytofusion device, using a BTX cuvette holder. Run the program with the following settings:
   a. Pre: 40v×30 see AC current
   b. Pulse: 300v×0.04 msec DC current
   c. Post: 40v×7 see AC current
12. After the fusion, incubate the cuvettes at 37° C. with 5% CO₂ for 20-30 minutes
13. Add the contents of cuvettes to 20 ml of HAT medium.
14. Use an electronic Matrix pipette to plate the fusion products at 50 μl/well into a 384-well cell culture plate
15. Incubate the plates at 37° C. with 5% CO₂ for 13-15 days prior to screening hybridomas for antibody production (see the Standard Human IgE Fluorescent ELISA protocol)

### (v) Subcloning of Hybridomas by Limiting Dilution

### Materials

1. Medium E (Stemcell Technologies, #03805)
2. 384-well cell culture plates (Nunc, 164688)
3. 48-well cell culture plates (Corning Inc. 3548)
4. Matrix electronic pipette 850 μl (Thermo Scientific 2014)
5. Matrix tips (Thermo Scientific 8042)

### Protocol

1. Enrichment dilution of the ELISA hits (option 1)
   a. Gently resuspend hits from a 384-well plate
   b. Place one drop of the cell suspension into a basin containing 21.5 ml of Medium E. Mix well
   c. Put the remainder of the cell suspension into one well of a 48-well plate containing 1 ml of Medium E
   d. Repeat for up to 5 hits; add the single drop of cells to the same basin and make individual cultures in the 48-well plate
   e. Plate 50 μl per well using an electronic Matrix pipette onto a 384-well plate
2. Enrichment dilution of the ELISA hits (option 2: a more stringent method of limiting dilution)
   a. Gently resuspend hits from a 384-well plate
   b. Place 1 μl of the cell suspension into a basin containing 20 ml of Medium E. Mix well
   c. Place 5 μl of the cell suspension into a separate basin containing 20 ml of Medium E. Mix well
   d. Place 10 μl of the cell suspension into a third basin containing 20 ml of Medium E. Mix well
   e. Plate the contents of each basin onto a separate 384-well plate at 50 μl per well
   f. Put the rest of the cell suspension into one well of a 48-well cell culture plate containing 750 μl of Medium E
3. Incubate the plates for 13-15 days at 37° C. with 5% CO₂, then recheck the 48-well plate and 384-well plates by ELISA
4. If no hits are found on the 384-well plate, repeat the enrichment dilution and plating of a 384-well plate if one or more of the 48-well cultures are active

### (vi) Subcloning Hybridomas by Flow Cytometry

### Materials

1. Medium E (Stemcell Technologies, #03805)
2. Flow cytometry tubes (Falcon 352235)
3. 48-well cell culture plates (Corning Inc. 3548)
4. 384-well cell culture plates (Nunc, 164688)
5. Hybridoma culture growing in a 384-well plate
6. Propidium iodide (Molecular Probes P-3566)

### Protocol

1. Gently resuspend a hit from a 48-well plate and place into a flow tube containing 1 ml of Medium E
2. Dispense 50 μl/well of Medium E onto on 384-well plate per hybridoma
3. Add 1 μl of propidium iodide to each tube of hybridomas
4. The flow core staff will process the samples, sorting 1 viable cell per well into 384-well plate
5. Incubate the plates at 37° C. with 5% CO₂ for 13-15 days
6. Screen the plates by ELISA or functional assay
7. If no hits are found on the 384-well plate, repeat the limiting dilution and plating of the 48-well culture hits or thaw frozen aliquot of that hybridoma line and repeat cloning procedure
   (vii) Thawing Hybridomas by Limiting Dilution Cloning

### Materials

1. 50 ml conical tubes (Falcon, 352070)
2. Medium A (Stemcell Technologies, #03801)
3. 384-well cell culture plates (Nunc, 164688)
4. Matrix electronic Pipette 850 μl (Thermo Scientific 2014)
5. Matrix tips (Thermo Scientific 8042)

### Protocol

1. Thaw an aliquot of the cells rapidly at 37° C. Remove stock from the water bath as soon as it has thawed
2. Drop wise, add 1 ml of warmed Medium A to the cells then gently transfer the cells to a 50 ml conical tube containing 10 ml of warmed Medium A
3. Centrifuge the cells for 5 minutes at 1100 RPM in a swinging bucket centrifuge
4. Discard the supernatant
5. Resuspend the cell pellet in 900 μl of Medium A
6. Prepare 5 different basins each containing 20 ml of Medium E
7. Into the 5 basins place 1 μl, 5 μl, 25 μl, 100 μl, and the remainder of the washed cells (one for each basin)
5. Plate the contents of each basin onto a separate plate at 50 μl per well using an electronic Matrix pipette
   (viii) Expanding Hybridomas

### Materials

1. 12 well cell culture plates (Falcon 353043)
2. Medium E (Stemcell Technologies, #03805)
3. T-75 Flasks (Falcon 353136)
4. T-225 Flasks (Falcon 353139)
5. Hybridoma Serum Free Media (Gibco 12045)
6. DMSO (Sigma D2650)
7. Cryovial tubes (Sarstedt 72.694.996)
8. Cell scrapers (Falcon 353087) or (Techno Plastic Products 99003)

### Protocol

1. Grow hybridoma culture in a 48-well plate in an incubator at 37° C. with 5% CO₂ until cells are 25% confluent
2. Check antibody production by ELISA (see the Standard Human IgE Fluorescent ELISA protocol)
3. Gently resuspend cells, and take an aliquot of cells for freezing (see the Freezing cells protocol)
4. Transfer the remainder of the cells to a 12 well plate containing 2 ml of Medium E
5. Grow 12 well plates in an incubator at 37° C. with 5% CO₂ until cells are 25% confluent
6. Check antibody production by ELISA (see Standard Human IgE Fluorescent ELISA protocol)
7. Freeze back an aliquot that represents 25% of the culture (see Freezing cells protocol)
8. Transfer the remainder of the cells in the 12 well plate to a T-75 flask and add Medium E to 30 ml
9. Every 3-5 days, feed the cells by aspirating off the old media and adding back fresh, warm media. Feed the cells every 3-5 days until the cells are 80% confluent
10. Mark 250 ml on four Corning T-225 flasks
11. Scrape cells off of the bottom of the T-75 flask using a cell scraper
12. Add the cell suspension to 1 L of Serum Free Media and divide equally to each of the four T-225 flasks
13. Freeze back an aliquot of the cells (see the Freezing cells protocol)
14. Add 30 ml of Medium E to the cells which remain in the T-75 Flask
15. Grow hybridomas in an incubator at 37° C. with 5% CO₂ in T-225 flasks for mAb production (see the chromatographic purification of full-length antibodies protocol) and T-75 flasks for RNA production
16. Freeze back 3 aliquot pellets of cells from the T-75 flasks for RNA production (see the Freezing cells protocol)
17. Grow the hybridomas in an incubator at 37° C. with 5% CO₂ in the T-225 flasks until cells are <10% viable using visual inspection
18. Harvest the medium for antibody purification by first centrifuging medium for 10 min at 2500 RPM followed by sterile filtration via 0.22 μm filter. Before purifying, perform an ELISA on the supernatant

### (ix) Freezing Hybridoma Cells

### Materials

1. Freezing Media
   a. 90% FBS (Sigma F-2442) or Medium E (Stemcell Technologies, #03805)
   b. 10% DMSO (Sigma D2650)
   c. Filter sterilize
2. 0.45 μm filter (Nalgene 167-0045)
3. Sarstedt cryovial tubes (Sarstedt 72.694.996)
4. Mr. Frosty freezing controlled freezing chamber

### Protocol

1. Label cryovials
2. Gently pipette the culture to resuspend any cells that have adhered to the bottom. When aspirating the cells, make sure to pipette up and down multiple times in a clockwise fashion around the side of the well, to ensure you really get the cells (even after doing this a few times, there are still some cells in the wells
3. Transfer cells to a cryovial tube and centrifuge in a tabletop centrifuge at 3000 RPM for 5 minutes
4. Discard supernatant and
   a. Option 1: slowly resuspend cells using 1 ml of freezing media
   b. Option 2: resuspend cells in 900 μl of FBS or Medium E and then slowly add 100 μl of DMSO
5. Place in a Mr. Frosty and put in the −80° C. freezer for at least 100 minutes (1 degree cooling per minute)
6. Store in liquid nitrogen
   (x) Isolation of Peripheral Blood Mononuclear Cells from Human Blood

### Materials

1. Na heparin green top blood collection tubes (BD Vacutainer 367874)
2. Serum red top blood collection tubes with clot activator (BD Vacutainer 367820)
3. 1× Sterile D-PBS (cellgro, 21-031-CM)
4. 50 ml conical tubes (Falcon, 352070)
5. Ficoll 1077 (Sigma 10771, Histopaque-1077)
6. Medium A (Stemcell Technologies, 03801)
7. Trypan blue (Gibco 15250-061)
8. DMSO (Sigma D2650)
9. Sarstedt cryovial tubes (Sarstedt 72.694.996)
10. Mr. Frosty controlled freezing chambers

### Protocol

1. Obtain peripheral blood from the subject by venipuncture. Have blood drawn into a Na heparin green top tube. If desired, have another aliquot drawn into a red top tube in order to freeze away an aliquot of subject sera (you may also save subject plasma in step 6). The approximate yield of peripheral blood mononuclear cells (PBMCs) is 1-2E6 cells/ml of peripheral blood
2. Add 15 ml of warmed 1×D-PBS to a 50 ml conical tube. One conical tube is needed for every 10 ml of blood drawn
3. Add 10 ml of blood to each 50 ml conical tube containing 1×D-PBS
4. Underlay the 25 ml of blood and D-PBS with 14 ml of warmed Ficoll
5. Centrifuge in a swinging bucket centrifuge for 25 minutes at 2500 RPM, with the brake and acceleration set to zero, or as low as possible
6. Remove and discard most of the plasma on top, down to about 2-3 mm from the buffy layer. Save 1 ml for testing, if desired (freeze plasma at −80° C.). Alternatively, blood can be collected into a red top tube
7. Remove buffy coat by tilting tube and removing cells until middle of liquid in tube starts to clear then pipette the material into a new 50 ml conical tube. Be sure to move the pipette around the sides of the tube in order to collect all PBMCs.
8. Add up to 50 ml of warmed Medium A to tube containing buffy coat layers
9. Centrifuge at 1800 rpm for 18 min in a swinging bucket centrifuge
10. Remove supernatant and resuspend cells in 2 ml of warmed Medium A for every initial 10 ml of blood
11. Add 10 μl of cells to 390 μl of trypan blue and count 2 quadrants
12. When continuing on to perform B cell cultures from the PBMCs without freezing see the B-cells from subject PBMCs protocol
13. For freezing PBMCs, resuspend cells at 5-10E6 cells per 900 μl in Medium A, then add 1/10final volume of DMSO
14. Freeze PBMCs in 1 ml aliquots in Cryovial tubes.
15. Place tubes in a Mr. Frosty freezing chamber and put in the −80° C. freezer for at least 100 minutes (1° C. cooling per minute)
16. Move samples to liquid nitrogen for storage

### (xi) Standard Human IgE Fluorescent ELISA

### Materials

1. Capture antibodies:
   Omalizumab (Xolair); 2.0 mg/ml
2. Secondary antibody
   Mouse anti-human IgE FC-HRP; Clone B3102E8-HRP; Southern Biotech; Cat No: 9160-05
3. Carbonate buffer
   Dissolve the following in 1 L of distilled water:
      i. 1.59 g Na₂CO₃
      ii. 2.93 g NaHCO
      iii. Adjust pH to 9.6
      iv. Filter solution at 0.22 μm
      v. Store at room temperature
4. 384 well; black, w/o lid; non-treated, non-sterile; Thermo Scientific No. 262260
5. ELx405 Plate Washer (Biotek)
6. Matrix Pipette (Thermo)
7. 64-channel multipipette (CappAero C10-64) or standard 12 channel pipette
8. QuantaBlu Fluorogenic Peroxidase Substrate Kits; Thermo Prod #15169
   QuantaBlu Substrate Solution, 250 ml
   QuantaBlu Stable Peroxide Solution, 30 ml
   QuantaBlu Stable Stop Solution, 275 ml
9. PBS 10× Molecular Biology Grade; Cellgro REF 46-013-CM
10. Block (1 L)
   100 ml of 10×PBS Molecular Biology Grade (Cellgro REF 46-013-CM)
   12-15 g of powdered milk (Great Value Instant Nonfat Dry Milk from Walmart)
   20 ml goat serum (Gibco 16210-072)
   Fill up to 1 L with dH₂O
   Add 500 μl of Tween 20 (Sigma P7949)
   Store at 4° C.
11. 1×Wash buffer (1 L)
   100 ml of 10×PBS Molecular Biology Grade (Cellgro REF 46-013-CM)
   1 ml of Tween 20 (Sigma P7949)
   900 ml water
   Store at room temperature
12. Medium A (Stemcell Technologies, 03801)
13. Molecular Devices Spectramax M3 (or equivalent fluorescence plate reader)

### Protocol

1. Dilute capture antibody in carbonate buffer for the number of plates you want to coat (make 10.5 ml per plate; there will be extra).
   a. Omalizumab (2 mg/ml); 1:1000
2. Coat plates overnight at 4° C.:
   a. Use 25 μl/well for a 384-well plate (10.5 ml)
   b. Note: If you forget to coat plates overnight, you can coat plates the same day at 37° C. for 3 hours
3. Wash each plate(s) 5 times with 1× wash buffer (or water) by running program 8 (384-5) on the 405.
   a. Alternatively, you can simply dump the contents into the sink and tap the surface of the ELISA plate on paper towels
4. Fill all wells with block:
   a. Use 115 μl/well for a 384-well plate (49 ml)
   b. Incubate at room temperature for at least 1 hour
      i. Don't shortcut this step
      ii. Block entire plate even if you aren't using every well
      iii. Start block first thing in the morning after the wash step
   c. Wash each plate(s) 5 times with 1× wash buffer (or water) by running program 8 (384-5) on the 405.
   d. Add block to all wells:
      i. Use 25 μl/well for a 384 well plate (10.5 ml)
5. Transfer 25 to 75 μl of rh-IL-21, CD40L, BAFF-NIH3T3 B-cell or hybridoma supernatant using a 12 channel pipette (if source pate is 96-well) or 64-channel multipipette (if source plate is 384-well)
   a. Perform this step in the laminar flow hood.
   b. Be careful not to suck up the rh-IL-21, CD40L, BAFF-NIH3T3 or B-cells using the pipette (don't pull supernatant when in contact with the bottom of the well)
6. Incubate plates for at least 30 minutes and up to one hour
   a. Always be sure to incubate the supernatants longer than the incubation time used for the secondary antibody
7. Wash each plate(s) 5 times with 1× wash buffer (or water) by running program 8 (384-5) on the 405.
8. Dilute the secondary antibody in block solution:
   a. Mouse anti-human IgE FC-HRP; Clone B3102E8-HRP
      i. Use 1:1000 dilution in block (1 μg/ml final)
      ii. Add 25 μl/well for 384 well plate (10.5 ml)
      iii. Add 100 μl/well for 96 well plate (10.5 ml)
   b. Incubate for 30 minutes at room temperature
      i. Note: Secondary antibodies conjugated to HRP are extremely difficult to get rid of
         1. Discard reservoir and tips that have come into contact with 2° HRP
9. Wash each plate(s) 7 times with 1× wash buffer by running program 9 (384-7) on the 405. Flip plate to opposite orientation and repeat for another 7 washes with 1× wash buffer.
10. Prepare fresh QuantaBlu Working Solution (WS) (WS is stable for 24 hrs at room temperature)
   a. Mix 9 parts of QuantaBlu Substrate Solution to 1 part of QuantaBlue Stable Peroxide Solution. Note: To reduce variability, equilibriate WS to RT before adding to the wells
   b. Prepare 10.5 ml of WS per plate:
      i. Add 9.45 ml QuantaBlu Substrate
      ii. Add 1.05 ml of QuantaBlu Stable Peroxide Solution
11. Add QuantaBlu Working Solution (WS) to each well and incubate at room temperature for 20-30 minutes
   a. Add 25 μl/well for 384 well plate (10.5 ml)
   b. Add 100 μl/well for 96 well plate (10.5 ml)
12. Stop peroxidase activity by adding 50 μl of QuantaBlu Stop Solution to each well
   a. Add 25 μl/well for 384 well plate (10.5 ml)
   b. Add 100 μl/well for 96 well plate (10.5 ml)
13. Measure relative fluorescence units (RFU) of each well with Molecular Devices Spectramax M3 (or equivalent fluorescence plate reader)
   a. The excitation and emission maxima for QuantaBlu Substrate are 325 nm and 420 nm respectively.
   b. Select Corning 384 well plate black as plate type
14. Transfer positive wells from the original culture plate to:
   a. If you were screening rh-IL-21, CD40L, BAFF-NIH3T3 activated B-cells, gently resuspend the positives cells and transfer each hit to microcentrafuge tube to prepare for cytofusion (see B-cell/HMMA fusion protocol)
   b. If you were screening hybridomas, transfer each hit to the next biggest well or flask containing Medium E (the order is 384-well plates to 48-well plates to 12-well plates to a T-75 flask to a T-225 flask)

### III. ACTIVE/PASSIVE IMMUNIZATION AND TREATMENT/PREVENTION OF ALLERGIC DISEASE

### A. Formulation and Administration

The present disclosure provides pharmaceutical compositions comprising engineered IgG antibodies and for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines are also envisioned where antibodies like those disclosed are produced in vivo in a subject at risk of peanut allergy. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### IV. ANTIBODY CONJUGATES

Antibodies of the present disclosure may be linked to at least one agent to from an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium⁹⁹m and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. IMMUNODETECTION METHODS

In still further embodiments, the present disclosure concerns immunodetection methods for binding, purifying, removing, quantifying and otherwise generally detecting peanut antigens.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of antibodies directed to specific epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing peanut allergens and contacting the sample with a first antibody in accordance with the present disclosure, as the case may be, under conditions effective to allow the formation of immunocomplexes.

These methods include methods for purifying allergens from a sample. The antibody will preferably be linked to a solid support, such as in the form of a column matrix, and the sample suspected of containing the allergen or antigen will be applied to the immobilized antibody. The unwanted components will be washed from the column, leaving the allergen antigen immunocomplexed to the immobilized antibody, which is then collected by removing the allergen or antigen from the column.

The immunobinding methods also include methods for detecting and quantifying the amount of allergen or antigen in a sample and the detection and quantification of any immune complexes formed during the binding process. Here, one would obtain a sample suspected of containing allergen or antigen and contact the sample with an antibody that binds the allergen or antigen, followed by detecting and quantifying the amount of immune complexes formed under the specific conditions. In terms of antigen detection, the biological sample analyzed may be any sample that is suspected of containing allergen or antiben, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to allergen or antigen present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the allergen antigen is added to the wells. After binding and washing to remove non-specifically bound immune complexes, the bound antigen may be detected. Detection may be achieved by the addition of another anti-allergen/antigen antibody that is linked to a detectable label. This type of ELISA is a simple “sandwich ELISA.” Detection may also be achieved by the addition of a second anti-allergen/antigen antibody, followed by the addition of a third antibody that has binding affinity for the second antibody, with the third antibody being linked to a detectable label.

In another exemplary ELISA, the samples suspected of containing the allergen or antigen are immobilized onto the well surface and then contacted with the anti-allergen/antigen antibodies of the disclosure. After binding and washing to remove non-specifically bound immune complexes, the bound anti-allergen/antigen antibodies are detected. Where the initial anti-allergen/antigen antibodies are linked to a detectable label, the immune complexes may be detected directly. Again, the immune complexes may be detected using a second antibody that has binding affinity for the first anti-allergen/antigen antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of anti-peanut allergen antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventors propose the use of labeled anti-peanut allergen antibodies to determine the amount of anti-peanut allergen antibodies in a sample. The basic format would include contacting a known amount of anti-peanut allergen monoclonal antibody (linked to a detectable label) with peanut allergen. The antigen or allergen is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probing. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### D. Immunodetection Kits

In still further embodiments, the present disclosure concerns immunodetection kits for use with the immunodetection methods described above. As the antibodies may be used to detect peanut allergen, or antibodies binding thereto, may be included in the kit. The immunodetection kits will thus comprise, in suitable container means, a first antibody that binds to an antigen, and optionally an immunodetection reagent.

In certain embodiments, the antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### E. Vaccine and Antigen Quality Control Assays

The present disclosure also contemplates the use of antibodies and antibody fragments as described herein for use in assessing the antigenic integrity of an antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, to the greatest extent possible, that they are efficacious in preventing or treating life-threatening disease, without themselves causing harm.

The increasing globalization in the production and distribution of vaccines has opened new possibilities to better manage public health concerns but has also raised questions about the equivalence and interchangeability of vaccines procured across a variety of sources. International standardization of starting materials, of production and quality control testing, and the setting of high expectations for regulatory oversight on the way these products are manufactured and used, have thus been the cornerstone for continued success. But it remains a field in constant change, and there is great pressure on manufacturers, regulatory authorities, and the wider medical community to ensure that products continue to meet the highest standards of quality attainable.

Thus, one may obtain an antigen or vaccine from any source or at any point during a manufacturing process. The quality control processes may therefore begin with preparing a sample for an immunoassay that identifies binding of an antibody or fragment disclosed herein to a viral antigen. Such immunoassays are disclosed elsewhere in this document, and any of these may be used to assess the structural/antigenic integrity of the antigen. Standards for finding the sample to contain acceptable amounts of antigenically intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, viral antigens may contain more than one protective epitope. In these cases, it may prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, may be determined.

### VI. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Research subjects. The protocol for recruiting and collecting blood samples from peanut allergic subjects was approved by the Vanderbilt University Medical Center Institutional Review Board (IRB 141330 and 142030). The inventor identified a panel of peanut allergic subjects in Tennessee who were diagnosed with food allergy (FA). The relevant clinical information is summarized in Table A. Diagnosis was based on clinical history and serum testing, when available, for the presence and quantity of IgE antibody to peanut. One hundred milliliters of blood was collected for adults and 10 mL for pediatric donors and processed to isolate PBMCs by density gradient separation on Ficoll. The cells were immediately cryopreserved and stored in liquid nitrogen.

Human hybridoma generation and IgE mAb purification. IgE-secreting human hybridomas were generated using methodology that was recently described in great detail (Wurth et al., 2018). Previously cryopreserved samples were thawed, washed, and counted before plating. For every 2 million viable cells, the following was added: 30 ml of prefusion medium (ClonaCell-HY 03801; Stemcell Technologies), 20 ml of CpG stock (2.5 mg/ml; ODN 2006), 1 ml each of mouse anti-human kappa (Southern Biotech; 9230-01) and mouse anti-human lambda (Southern Biotech; 9180-01), and 1 million gamma-irradiated NIH3T3 fibroblast line genetically engineered to constitutively express cell-surface human CD154 (CD40 ligand), secreted human B cell activating factor (BAFF) and human IL-21. The mixture then was plated into 96-well flat bottom culture plates at 300 ml/well and incubated at 37° C. with 5% CO₂ for 7 days, prior to screening for IgE secretion using an ELISA. Omalizumab was used as a capture antibody, coating 384-well black ELISA plates at a concentration of 10 mg/ml. After blocking, 100 ml of supernatant was transferred from each well of the 96-well plates containing B cell lines, using a VIAFLO-384 electronic pipetting device (Integra Biosciences). Secondary antibody (mouse anti-human IgE Fc; Southern biotech, 9160-05) was applied at a 1:1,000 dilution in blocking solution using 25 ml/well. After 10 washes with PBS, fluorogenic peroxidase substrate solution (QuantaBlu; Thermo Scientific 15162) was added at 25 ml/well, as per manufacturer instructions. Relative fluorescence intensity was determination on a Molecular Devices plate reader. Wells are counted as positive if the relative fluorescence intensity is >5 times background. IgE B cell frequencies then are expressed as the number of IgE positive wells per 10 million peripheral blood mononuclear cells. A secondary screen, by ELISA using commercial peanut extract (ALK-Abello), was performed to allow for the identification of peanut specific B cell cultures in some cases.

HMMA2.5 non-secreting myeloma cells were counted and suspended in cytofusion medium composed of 300 mM sorbitol, 1.0 mg/ml of bovine serum albumin, 0.1 mM calcium acetate, and 0.5 mM magnesium acetate. Cells from IgE positive wells were pipetted gently into microcentrifuge tubes containing 1 ml of cytofusion medium. B cells and HMMA2.5 cells were washed three times in cytofusion medium to ensure equilibration. HMMA2.5 cells were then suspended in cytofusion medium to achieve a concentration of 10 million cells/ml. The HMMA2.5 cell suspension was added to each sample tube and the mixture pipetted into cuvettes (BTX, 450125). Cytofusion was performed using a BTX cuvette holder (BTX Safety stand, model 630B) with a BTX ECM 2001 generator (BTX; 45-0080) programed to run with following settings: a prefusion AC current of 70 V for 40 s, followed by a DC current pulse of 360 V for 0.04 ms and then a post-fusion AC current of 40 V for 9 s. After fusion the content of each cuvette was then added to 20 ml of hypoxanthine-aminopterin-thymidine (HAT) medium containing ouabain, composed of the following: 500 ml of post-fusion medium (Stemcell Technologies, 03805), one vial 50×HAT (Sigma, H0262), and 150 ml of a 1 mg/ml stock of ouabain (Sigma, 013K0750). Fusion products then were plated into 384-well plates and incubated for 14 days before screening hybridomas for IgE antibody production by ELISA.

Wells containing hybridomas producing IgE antibodies were cloned biologically by indexed single cell flow cytometric sorting into 384-well culture plates. Once clonality was achieved, each hybridoma was expanded in post-fusion medium in 75-cm² flasks. IgE mAb was expressed by large-scale growth of the hybridoma in serum free medium (Gibco Hybridoma-SFM; Invitrogen, 12045084) in 225-cm² flasks. IgE antibody was then purified by immunoaffinity chromatography (Omalizumab covalently coupled to GE Healthcare NHS activated HiTRAP; 17-0717-01) and visualized by SDS-PAGE for purity.

Peanut allergen specificity and EC₅₀ assays by ELISA. The final allergen specificity of each IgE mAb was confirmed/defined using Thermo/Phadia ImmunoCAP. Medium from cultured IgE secreting human hybridoma clones were used to measurement on ImmunoCAP devise—performed at the Johns Hopkins Allergy and Clinical Immunology Reference Laboratory.

Half maximal effective concentration (EC₅₀s) were obtained for peanut allergen protein binding of human IgE mAbs. Peanut allergens Ara h 1, 2, 3 and Ara h 6 were expressed in E coli with a 6×His-tag and purified using nickel chromatography. Allergen protein then was used to coat 384-well ELISA plates at a concentration of 25 mg/ml. After blocking with 2% cow's milk for 1 h, 25 ml of IgE antibody was added as a dilution series in triplicate, starting at a concentration of 10 mg/ml. After a 1 h incubation at room temperature and washing five times, secondary antibody (mouse anti-human IgE Fc; Southern biotech, 9160-05) was applied at a 1:1,000 dilution in cow's milk blocking solution using 25 ml/well. After 10 washes with PBS, fluorogenic peroxidase substrate solution (QuantaBlu; Thermo Scientific 15162) or TMB (Thermo, 34029) was added at 25 ml/well, as per manufacturer instructions. Relative fluorescence intensity or optical density was determination on a Molecular Devices plate reader.

Immunoprecipitation and mass spectroscopy. Human IgE mAbs that demonstrated binding to peanut extract in ELISA or ImmunoCAP and/or Western blot analysis but did not bind the recombinant major peanut allergen proteins, were used for immunoprecipitation (IP). Each purified mAb was covalently coupled per the manufacturer's instructions to magnetic microbeads (Invitrogen Dynabeads: 14311D). Using peanut extract diluted 50% in PBS, IP was performed in parallel with an irrelevant IgE mAb acting as a control. Eluted target protein was then identified using mass spectrometry proteomics analysis. The target protein was confirmed if there was a peanut (Arachis hypogaea) protein present in the elution of the unknown IgE mAb that was ≥10 times the total spectrum count of the same protein from the elution of the control mAb.

Peanut allergen competition by ELISA. IgE mAbs which represent an immunodominant antigenic site were expressed as IgG switched variant antibodies. Purified IgG antibody was used to coat 384-well ELISA plates at a concentration of 25 mg/ml. Plates were blocked with 2% cow's milk for 1 h. Peanut allergens were expressed in E coli with a 6×His-tag and purified using nickel chromatography. Allergen protein then was added to ELISA plates at a concentration of 25 mg/ml in blocking buffer, to allow for IgG antibody capture. After washing, 25 ml of IgE antibody was added as a dilution series in triplicate, starting at a concentration of 10 mg/ml. After a 1 h incubation at room temperature and washing five times, secondary antibody (mouse anti-human IgE Fc; Southern biotech, 9160-05) was applied at a 1:1,000 dilution in cow's milk blocking solution using 25 ml/well. After 10 washes with PBS, fluorogenic peroxidase substrate solution (QuantaBlu; Thermo 15162) or TMB (Thermo, 34029) was added at 25 ml/well, as per manufacturer instructions. Relative fluorescence intensity or optical density was determination on a Molecular Devices plate reader. Competition was said to occur if the area under the curve of the IgE antibody binding is reduced by >75% from that of the same IgE antibody binding directly to its allergen target protein. Competition was said to not be occurring if the area under the curve of the IgE antibody binding is reduced by <25% from that of the same IgE antibody binding directly to its allergen target protein.

Passive systemic anaphylaxis human FcεRI transgenic mice. Mice were maintained under specific pathogen-free conditions and used in compliance with the revised Guide for the Care and Use of Laboratory Animals (National Academies Press, 2011). These mice with 2 gene mutations express the human Fc of IgE, high affinity I, receptor for α polypeptide (FCER1A), under the control of the human FCER1A promoter and carry the mutation targeted for Fcεr1a^{tmlKnt} (Dombrowicz et al., 1996). Mice that are hemizygous for the transgene and homozygous for the targeted deletion of the mouse FcεRI respond to experimental induction of anaphylaxis with human IgE. Eight-week-old mice are sensitized passively by IP injection with 100 μg total of purified human IgE mAb(s), three days prior to challenge. In experiments involving therapeutic blocking of antigenic sites with IgG switched variant antibodies, mice are simultaneously injected with 1 mg total purified antibody, at the time of IgE sensitization. Implanted temperature probes then are placed subcutaneously along the back of the mice. Mice are challenged with 500 μl of 10% peanut extract via IP injection (ALK-Abello), diluted in sterile PBS. Alternatively, mice are challenged with 500 μl of purified recombinant allergen(s) diluted in sterile PBS. Temperature is then monitored in five minute increments to define the severity of anaphylaxis. Temperatures of sensitized and sham-sensitized mice following allergen challenge were compared independently for each allergen challenge and at each time point using paired 2-tailed t-test assuming unequal variance. Time points with calculated P-values less than 0.05 were considered significant. Error bars for the mouse temperature measurements represent SEM.

Variable gene sequencing of human peanut specific IgE mAbs. Total RNA was extracted from 1 million clonal IgE-expressing human hybridoma cells (RNeasy kit, Qiagen: 74104). Reverse transcription PCR (RT-PCR) then was performed for 30 cycles with a 5′ primer set described previously (Smith et al., 2009) and a 3′ primer specific to the IgE constant using the OneStep RT-PCR kit (Qiagen: 210210). Following gel purification, the cDNA product was cloned into pCR2.1 using a TA cloning kit (Invitrogen: 45-0046). Antibody genes were Sanger sequenced and analyzed using the IMGT database, world-wide-web at imgt.org.

A prototype site-specific IgE mAb found to target a major peanut allergen protein was selected for recombinant expression as an IgG1 isotype switched variant immunoglobulin. Specifically, total RNA from hybridomas is used in RT-PCR reactions using previously described primer sets (Smith et al., 2009). This has been performed for all peanut IgE mAbs listed in Table C and D. VH/VL sequences are cloned into IgG1 mammalian expression vectors for recombinant production of switched variant mAbs. Plasmid DNA containing heavy and light chains then will be co-transfected transiently into HEK 293 cells for expression (Invitrogen; R79007) and mAb purified using affinity chromatography with protein G (GE Healthcare HiTRAP; GE17-0404). Each purified mAb is subjected to a battery of tests to confirm its authenticity by comparing head to head the binding properties of the recombinant antibody to those of the original hybridoma expressed IgE antibody. They are then used as molecular tools for competition assays, serum-blocking studies, to interfere with peanut allergen-specific IgE-mediated anaphylaxis in mice, and to make FAb for structural studies. The inventor has expressed and purified gram quantities of IgG antibody for many of the prototype site-specific IgE mAbs shown in Table C (those highlighted in red).

### Example 2—Results

Identification of ultra-rare human peanut allergen specific IgE mAbs. Using peanut allergic patient IgE serum profiles and skin testing results, obtained by their allergist, the inventor was able to identify thirteen subjects which had high frequencies of circulating IgE encoding B cells—see Table A. Cryopreserved PBMCs were thawed, grown in 96 well cultures, and screened for the IgE isotype to allow for identification of desired B cell clones. A secondary screen, by direct ELISA using peanut extract was also performed in many cases to allow for the identification of peanut specific B cells in culture. An approximate minimum B cell frequency (#IgE expressing cultures/total #PBMCs cultured) were calculated for each subject. Growth and screening of B cells from these peanut allergic subjects revealed that on average their IgE encoding B cell frequency was approximately 6 cells in 10 million peripheral blood mononuclear cells. Remarkably, secondary screening of the same B cell cultures when performed revealed, on average, a peanut specific IgE encoding B cell frequency of only 3.6 B cells per 10 million peripheral blood mononuclear cells. A total of 94 human hybridomas secreting IgE antibody were obtained from peanut-allergic subjects, an average of 7 hybridomas per allergic subject. Finally, screening of purified IgE mAbs obtained from these subjects' hybridomas identified 48 which were specific for allergens contained within peanut extract—shown in Table C.

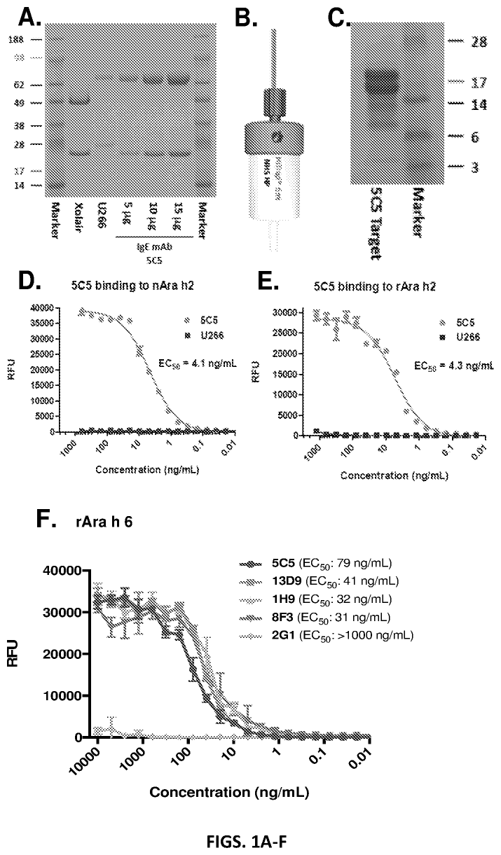

Verification and validation of IgE mAbs peanut allergen specificity. Following the unbiased generation of IgE secreting human hybridomas from peanut allergic subject PBMCs, peanut protein binding and peanut allergen protein specificity was determined. Hybridoma culture supernatant was initially used for this validation. As can be seen in Table B, peanut ImmunoCAP (F13) was used to verify binding to a peanut antigen. Using the available ImmunoCAP components (purified allergen proteins: Ara h 1, F422; Ara h 2, F423; Ara h 3, F424; Ara h 8 F352; Ara h 9 F427), the exact allergen specificity was determined for those which bound Ara h 1, 2, and 3. Nearly all IgE mAbs which could not be determined by this method were found to be specific to Ara h 6. As shown in FIG. 1F, recombinant Ara h 6 was produced and purified to confirm IgE mAb specificity.

Human IgE mAbs were expressed by large scale growth in serum free medium and purified using Omalizumab immunoaffinity chromatography (see FIG. 1A). Major peanut allergen proteins Ara h 1, 2, 3, and 6 were expressed in E. coli and purified using nickel chromatography. Peanut allergens were also purified using human IgE mAb, linked to chromatography resin using amine coupling, allowing for immunopurification from peanut extract. As can be seen in FIGS. 1A-F, E. coli expressed recombinant peanut allergen Ara h 2 was bound by human IgE mAb 5C5 in EC₅₀ assays identically to the naturally-occurring peanut allergen Ara h 2, showing the authenticity of the recombinant protein. Nearly all of the human IgE mAbs which bound to peanut, but not the peanut components, using ImmunoCAP, were found to bind recombinant Ara h 6—see FIG. 1F for example of EC₅₀.

Mapping antigenic sites of the major peanut allergen proteins by competition. A very important concept at the heart of allergic disease, and the understanding of functional mapping studies being performed, is the antigenic site (a non-overlapping antigenic region). In order for cross-linking of Fcε receptors to occur with a native monomeric allergen protein, two different IgE antibodies must bind simultaneously—this is not the case, in theory, for some multimeric allergen proteins. This implies that the two antibodies must be directed toward different antigenic sites on the same allergen protein. Thus, in vivo, to cause anaphylaxis, one must have two different IgE antibodies directed against two different antigenic sites of Ara h 6 for example—one IgE antibody alone could not result in Fcε receptor cross-linking by the Ara h 6 molecule. In vitro, antigenic sites can be easily defined using antibody competition assays. Classically, this is done using ELISA and is a preferred method of evaluation.

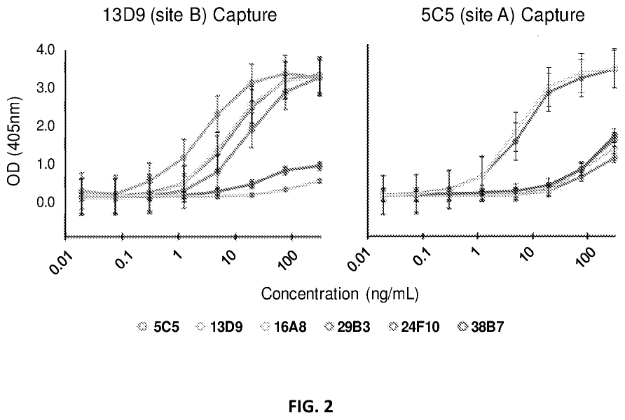

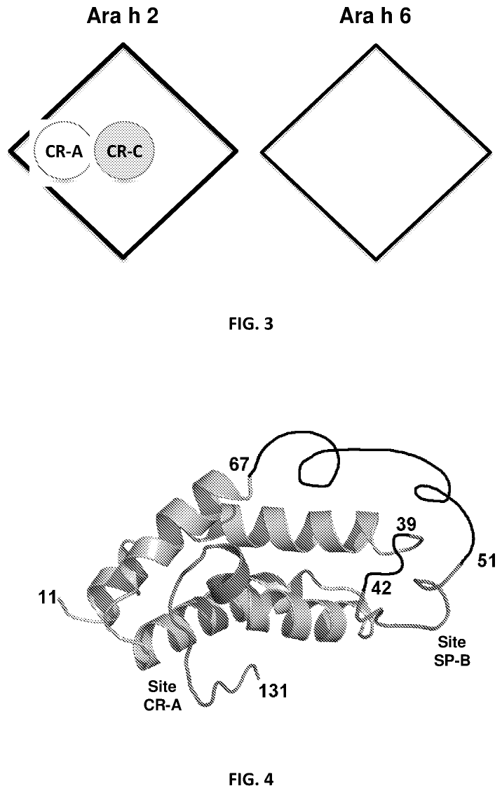

Antigenic sites are defined by competition assays using ELISA. See FIG. 2 for an example of antigenic mapping with mAbs in the inventor's peanut panel (Table B) using competition ELISA. Antibody specific to Ara h 2 site A is used to capture recombinant Ara h 2, if a second antibody is not able to bind simultaneously, it is said to compete for the same antigenic site. If two antibodies can bind the recombinant allergen simultaneously, they do not compete, and thus bind to different spots on the allergen protein. The results of the inventor's comprehensive competition analysis are summarized diagrammatically in FIG. 3. Ara h 2 contains three immunodominant antigenic sites A, B, and C. Antibodies to these sites primarily cross-react (CR) with Ara h 6, with the exception of specific site (SP) B, defined by IgE mAb 38B7. Antibodies which bind primarily to Ara h 6, with weak or no cross-reactivity to Ara h 2, also bind three distinct immunodominant antigenic sites on Ara h 6 (sites A, B, and C). For each competition group, for each major allergen protein, prototype IgE mAbs were selected to represent the population. These prototype mAbs, highlighted in red in Table C, were expressed as recombinant IgG1 switched variant antibodies. These antibodies are used as key tools for competition assays and various mapping approaches, such as serum blocking and skin test blocking studies.

Peptide microarray. The inventor used peptide arrays to help determine the approximate locations of the antigenic sites targeted by his human IgE mAbs. In collaboration with Dr. Hugh Sampson at Mount Sinai he tested Ara h 2-specific IgE mAbs using a Luminex peptide array technology (Shreffler et al., 2004). This has led to the identification of the approximate locations of both Ara h 2 site CR-A and SP-B, see FIG. 4 for graphic illustration summarizing these results. Most of the IgE mAbs did not bind any peptide in the array, suggesting they strictly bind conformational epitopes. Several of the Ara h 2 site CR-A-specific IgE mAbs, however, bound strongly to peptide LPQQCGLRAPQRCDL at the C-terminus of the allergen protein. Ara h 2 site SP-B-specific antibody 38B7, which competes with all Ara h 2 site CR-B antibodies, bound to two peptides DSYERDPYSPSQDPY and PYSPSQDPYSPSPYD. Interestingly, the crystal structure of Ara h 2 was determined using a maltose-binding protein (MBP) fusion protein (Mueller et al., 2011). In that paper the authors used the fusion protein to define an immunodominant population of IgE antibodies in the sera of peanut allergic subjects, which is the population the inventor defines here as site CR-B, the immunodominant antigenic site of Ara h 2. This region has also been described previously as being an immunodominant target of the Ara h 2 and Ara h 6 IgE antibody response seen in human sera (Chen et al., 2016). These peptides make up the disordered loop between helices 2 and 3 of Ara h 2, the region that differs between Ara h 2.01 and Ara h 2.02 isoforms, not present in the crystal structure due to its high degree of flexibility. This region is on the opposite end of the molecule and explains why site CR-A and CR-B IgE mAbs are capable of cross-linking FcεRI in the presence of Ara h 2 and cause such profound anaphylaxis in mice, as described below.

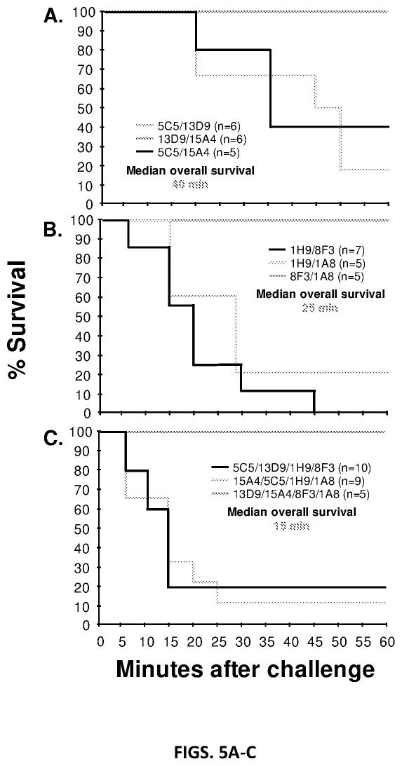

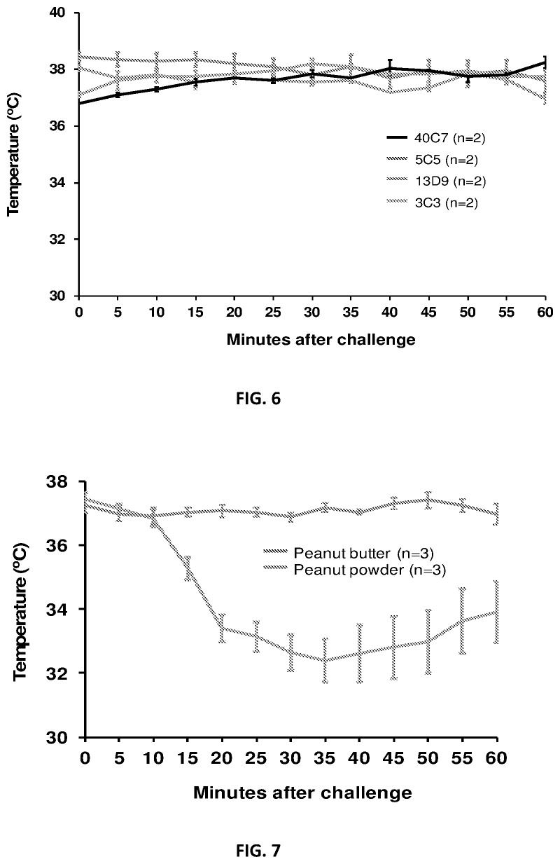

Testing functional activity of peanut allergen-specific human IgE pairings in animals. IgE mAbs which bind to different antigenic sites on the same allergen are studied for functional activity in a mouse model of passive systemic anaphylaxis. The results of competition assays and EC₅₀ measurements allow for the strategic selection of IgE mAbs to be assessed by passive anaphylaxis using human FcεRI transgenic mice. Human FcεRI transgenic mice (B6.Cg-Fcεr1α^{tm1Knt}Tg(FCER1A) 1Bhk/J were purchased from The Jackson Laboratory (stock #010506), brought out of cryogenic storage, bred and genotyped. Anaphylaxis in mice is characterized by hypothermia (Osterfeld et al., 2010). The inventor was able to use these mice to quantify the ability of human IgE mAb(s) to incite anaphylaxis upon challenge with peanut extract or purified allergen proteins (see FIGS. 5A-C and FIG. 6).

Mice sensitized using proposed functional sets of human IgE antibodies, as determined by antigenic site mapping, are assessed for their ability invoke peanut-induce anaphylaxis. Mice are sensitized passively by intraperitoneal (IP) injection with 100 μg total of purified human IgE mAb(s) three days prior to challenge in order to upregulate the transcription and expression of the human FcεRI α-chain (Smrz et al., 2014; Beck et al., 2004). See FIGS. 5A-C for results of experiments showing how this model can be used to validate the in vitro antigenic mapping. In each panel of FIGS. 5A-C, IgE mAbs which bind the same antigenic site on the same allergen protein do not induce anaphylaxis. As can be seen in FIG. 5A, mice sensitized with a pair of human IgE mAbs which bind different antigenic sites on Ara h 2, 5C5 and 13D9, exhibited significant anaphylaxis, leading to a median time of death of 45 min. However, as predicted by mapping, mice that were sensitized with 13D9 and 15A4 show no sign of anaphylaxis because these two Ara h 2-specific mAbs bind the same antigenic site (they are in the same competition group) and are thus not capable of cross-linking FcεRI. This also can be seen with mAbs to Ara h 6 (see FIG. 5B), and using the combination of mAbs to Ara h 2 and Ara h 6 (see FIG. 5C). A median overall survival of 15 min is seen (FIG. 5C) when two functional pairings directed against Ara h 2 and 6 are combined (mAbs 5C5, 13D9, 8F3, and 1H9). This model is exceptional for such analyses as it has a very broad dynamic range. Mice sensitized with functional pairs of Ara h 1 or Ara h 3 mAbs, for example, do not have a fatal outcome, they exhibit a maximum drop in temperature of approximately 6-degrees (data not shown). The results presented are from mice challenged with 500 μl of 10% peanut extract via IP injection (ALK-Abello). The inventor sees similar results when purified natural and recombinant allergens are used. The inventor does not see anaphylaxis occurring in mice sensitized with a single IgE mAb to any peanut allergen protein (FIG. 6), emphasizing the importance of antigenic mapping and the coordination between populations of antibodies within the allergic human to cause allergy severity. By injecting mice IP, the inventor was able to control dosing, allowing us to assess whether two IgE mAbs are able to function in cross-linking the IgE receptor or not and quantify their degree of function. Thus, this mouse model is of great value for functionally mapping human IgE mAbs, allowing for functional comparisons between antibody groups and structural data.

The inventor also was able to induce anaphylaxis via oral challenge with peanut (as mucosal absorption is thought to play a major role; see Dirks et al., 2005), though the results are much less dramatic and the dosing is much more difficult to control—see FIG. 7. While mice do not die when sensitized with mAbs 5C5, 13D9, 8F3, and 1H9 there was still a 5-degree temperature drop as a result of the severe anaphylactic reaction. What can also be seen is that the preparation of peanut is essential when given orally as a 100 μl slurry. Freshly prepared peanut butter, made by crushing dry roasted peanuts in water with a mortar and pestle, resulted in anaphylaxis. The inventor can induce anaphylaxis by feeding peanuts to mice, breaking the long held dogma that this is not possible, which was based previously on inducing IgE in mice.

Variable gene sequence germline usage and mutation rate of human peanut specific IgE mAbs. As can be seen in Table D, the sequences of the inventor's human anti-peanut IgE antibodies are unique, use different germline genes, have variable length CDR3 sequences, and frequently have a substantial number of mutations. Remarkably human antibodies to peanut allergens frequently possess very high rates of mutation, suggesting that repeated allergen exposure results in repeated bouts of somatic hypermutation in peanut allergen-specific B cells. The total number of nucleotide mutations from their respective germline sequences for the heavy and light chains of Ara h 2-specific antibodies 15B8 and 16G12, for example, are 69 and 63 respectively. These unique IgE sequences will provide the allergen-specific reference needed to interrogate sequencing datasets and allow for further discovery of human antibodies to peanut and to define the origin of the IgE, via direct or indirect isotype class-switching in B cell development.

Serum blocking assays to quantify unique subpopulations of IgE using ImmunoCAP. Serum-blocking analyses makes use of the inventor's switched variant IgG mAbs ability to block serum IgE from being measured in ImmunoCAP and/or ELISA. This data, which is essentially quantification of each human subject's unique subpopulations of serum IgE, can then be used in conjunction with that subject's clinical information, and correlates drawn. This information allows for the creation of a complete, comprehensive, and clinical phenotypic map of the human anti-peanut IgE antibody response.

The best way to determine the functional significance of antigenic groups of peanut-specific IgE antibodies made by allergic subjects is to perform serum-blocking analyses. This not only allows for the determination of immune dominance within an individual, but also within the peanut allergic population as a whole. The inventor compiled all of the competition data from his panel of peanut IgE mAbs, see Table C for designated antigenic sites—this information is displayed graphically in FIG. 3. Interestingly, the mAbs that predominantly target Ara h 2 have some degree of in vitro cross-reactivity toward Ara h 6 and thus the antigenic sites are labeled accordingly. However, mAbs which target Ara h 6 are more specific. Many Ara h 6-specific mAbs do not bind to Ara h 2 at any concentration (in ImmunoCAP and/or ELISA), while others bind Ara h 2 with an EC₅₀>100× the concentration for binding to Ara h 6. He has not found any relevant cross-reactive binding between Ara h 2 or 6 specific mAbs and Ara h 1 and/or 3. Prototype IgE mAbs representing each antigenic group have been expressed/purified as switched variant IgG mAbs, allowing for blocking studies to be performed.

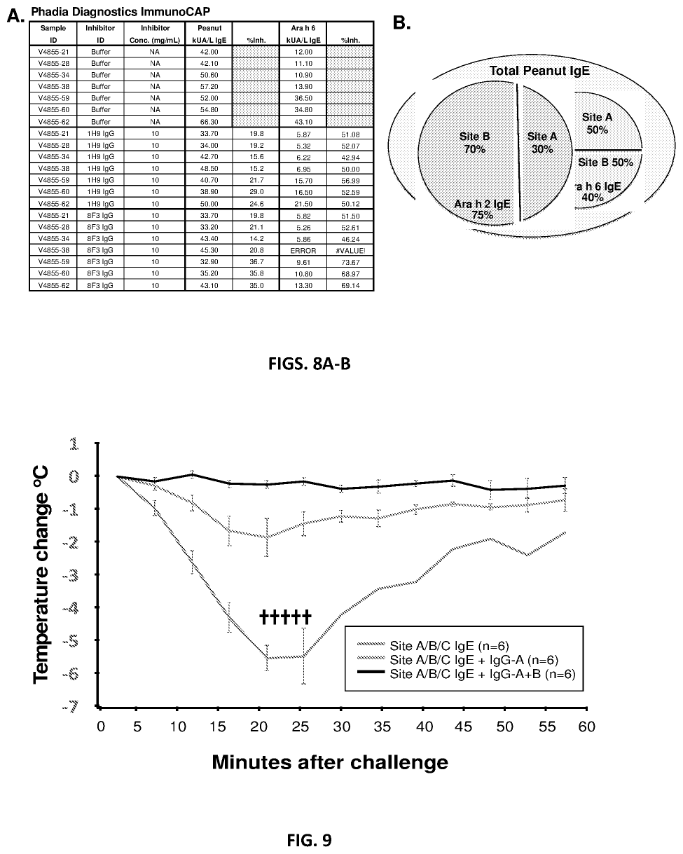

Using seven peanut allergic subjects' frozen serum samples, the inventor performed blocking studies using Ara h 2- and Ara h 6-specific IgE ImmunoCAP. These studies were performed by first making 190 μl aliquots of each serum sample. To each aliquot, 10 μl of Ara h 2 or 6 site-specific IgG (at 20 mg/mL concentration) or PBS is added. Ara h 2 and Ara h 6-specific IgE ImmunoCAP measurements then are performed on each aliquot of each serum sample. The PBS control measurement provides the total IgE that subject makes against Ara h 2 and 6. Each sample containing an IgG will provide a measurement that represents the amount of IgE not blocked by that site-specific IgG. The preliminary results are fascinating and help support the hypothesis that the human anti-peanut allergen IgE serum response is made up of a restricted number of antigenic site-specific groups of antibodies. The results for Ara h 6 are shown in FIG. 8A. Approximately 50% of each individual's Ara h 6-specific IgE were directed toward antigenic site A and 50% against site B. For peanut-specific binding in general, each site blocked approximately 20-25% of the total peanut binding IgE from each subject's serum. Summarizing the results for Ara h 2: approximately 75% of the sera showed 60% of their Ara h 2 IgE was directed toward site CR-B, while 40% was toward site CR-A; approximately 25% of the sera showed 60% of their Ara h 2 IgE was directed toward site CR-B, while 40% was toward an unidentified site (likely site CR-C). This date suggests that site CR-B is the primary immunodominant site of Ara h 2, recognized by all peanut allergic subjects' serum IgE; thus, blocking it would eliminate all Ara h 2-mediated degranulation as the other sites could not cross-link the receptor on their own. FIG. 8B shows the results of peanut allergic subject serum blocking studies summarized in a vin diagram.

Testing therapeutic effect of blocking peanut allergen induced anaphylaxis in animals. The inventor tested whether isotype switched variant IgG mAbs can be used to block passive systemic anaphylaxis induced in mice. Mice were sensitized using the highly functional set of human IgE antibodies directed against Ara h 2, which fully cross-react with Ara h 6, representing sites CR-A, CR-B, and CR-C (see FIG. 9). Mice were sensitized passively by intraperitoneal (IP) injection with 100 μg total of the purified human IgE mAb(s), with or without IgG blocking antibody, three days prior to challenge. Mice that did not receive any IgG blocking antibody had severe rapid anaphylaxis following challenge with 10% peanut extract, where 5 out of 6 mice died within 25 minutes. When mice received a single IgG blocking mAb specific for CR-A, mice had a slight drop in temperature following peanut challenge, approximately two-degrees at 20 minutes. Mice that received two IgG blocking mAbs, representing CR-A and CR-B, had no significant drop in temperature and exhibited no visible evidence of anaphylaxis following peanut challenge.

TABLE A
Subject demographics and hybridoma yield

| | | | | | | | | Peanut- | |
|---|---|---|---|---|---|---|---|---|---|
| | | | | | Peanut- | | IgE B-cell | specific | |
| | | | | Total | specific | Peanut | frequency | IgE B-cell | IgE |
| Subject | | | Allergic | serum IgE | Serum IgE | SPT (mm | (per 10⁷ | frequency | hybridomas |
| code | Age | Sex | disease | (kU IgE/L) | (kUA/L) | wheal) | PBMCs) | (per 10⁷ PBMCs) | generated |
| P1 (32) | 18 | M | AF, Asthma | 677 | 65.1 | 20 × 15 | 12 | 8.4 | 43 |
| P2 (36) | 5 | M | AF, AD | 22497 | ND | 16 × 15 | 9 | 6.7 | 3 |
| P3 (92) | 5 | M | AF, AD | ND | >100 | 16 × 10 | 7 | ND | 3 |
| P4 (126) | 13 | F | AF | ND | ND | 45 × 30 | 9 | 3.0 | 3 |
| P5 (129) | 10 | M | F, Asthma, A | 803 | 73.7 | 8 × 11 | 7 | 2.9 | 5 |
| P6 (137) | 4 | M | AF, AD | 2713 | >100 | 6 × 4 | 5 | 0.3 | 4 |
| P7 (144) | 4 | M | F, Asthma, A | ND | ND | ND | 3 | 1.2 | 6 |
| P8 (154) | 10 | M | AF, Asthma | ND | >100 | 24 × 24 | 8 | 4.6 | 9 |
| P9 (170) | 27 | M | AF | ND | ND | ND | 3 | ND | 8 |
| P10 (180) | 59 | F | AF, Asthma | 1396 | ND | ND | 11 | ND | 5 |
| P11 (181) | 15 | M | AF, Asthma | 1615 | >100 | ND | 6 | ND | 2 |
| P12 (195) | 6 | F | AF | 351 | >100 | 24 × 45 | 2 | ND | 1 |
| P13 (205) | 9 | F | AF | ND | >100 | 19 × 40 | 8 | 1.9 | 2 |

Subject age, total serum IgE, and peanut-specific IgE serum quantification is shown. IgE B cell frequencies is expressed as the number of IgE positive cells per 10 million peripheral blood mononuclear cells. The total IgE expressing human hybridomas generated for each subject is listed. AF, adverse food reaction; AD, atopic dermatitis; SPT = skin prick test; ND = not determined.

TABLE B
Human peanut-specific IgE mAb ImmunoCAP analysis

| | Peanut | Ara h1 | Ara h2 | Ara h3 | Ara h8 | Ara h9 |
|---|---|---|---|---|---|---|
| | F13 | F422 | F423 | F424 | F352 | F427 |
| IgE mAb | kUA/L | kUA/L | kUA/L | kUA/L | kUA/L | kUA/L |
| 3C3 | 3138.00 | 0.60 | 0.85 | 3462.00 | 0.51 | 0.46 |
| 26C4 | 7.86 | <0.1 | <0.1 | <0.1 | <0.1 | <0.1 |
| 21C10 | 715.00 | 0.36 | 0.46 | 0.83 | 0.53 | 0.32 |
| 3B7 | 1502.00 | 0.30 | 0.63 | 1.15 | 0.42 | 0.25 |
| 22F2 | 298.00 | <0.1 | 0.16 | 0.14 | 0.11 | <0.1 |
| 4D11 | 79.50 | 0.12 | 0.14 | 0.19 | 0.17 | <0.1 |
| 7B6 | 420.00 | 0.19 | 0.41 | 0.32 | 0.29 | 0.21 |
| 38B7 | 286.00 | 0.11 | 203.00 | 0.23 | 0.15 | 0.11 |
| 26A5 | 783.00 | 0.15 | 0.46 | 0.25 | 0.15 | 0.14 |
| 3F9 | 326.00 | 0.13 | 0.14 | 0.17 | 0.16 | <0.1 |
| 6E10 | 1108.00 | 0.15 | 0.28 | 0.34 | 0.21 | 0.15 |
| 2C9 | 21.40 | <0.1 | 0.13 | <0.1 | <0.1 | <0.1 |
| 15A7 | 14.00 | <0.1 | 25.80 | 0.14 | <0.1 | <0.1 |
| 4G4 | 1170.00 | 1663.00 | 0.26 | 0.48 | 0.34 | 0.19 |

Hybridoma cell culture supernatant was used for initial identification of allergen specificity using ImmunoCAP analysis. Peanut reactivity was first determined using peanut ImmunoCAP. Component analysis identified IgE antibodies specific for Ara h 1, 2, and 3. All antibodies not identified in this table were determined to bind Ara h 6.

TABLE C
Human peanut-specific IgE mAbs

| | | | | Allergen | |
|---|---|---|---|---|---|
| Human | IgE mAb | Fine | Antigenic | size | Allergen |
| IgE mAbs | reactivity | specificity | site | (kDa) | family |
| 40C7 | Peanut | Ara h 1 | Site A | 65 | Vicilin |
| 4G4 | Peanut | Ara h 1 | Site A | 65 | Vicilin |
| 3B10 | Peanut | Ara h 1 | Site B | 65 | Vicilin |
| [symbol] | Peanut | Ara h 2 | Site CR-A | 17 | Conglutin |
| 32G9 | Peanut | Ara h 2 | Site CR-A | 17 | Conglutin |
| [symbol] | Peanut | Ara h 2 | She CR-A | 17 | Conglutin |
| 29B3 | Peanut | Ara h 2 | Site CR-A | 17 | Conglutin |
| 24F10 | Peanut | Ara h 2 | Site CR-A | 17 | Conglutin |
| 14B3 | Peanut | Ara h 2 | Site CR-A | 17 | Conglutin |
| 15A7 | Peanut | Ara h 2 | Site CR-A | 17 | Conglutin |
| 2F12 | Peanut | Ara h 2 | Site CR-A | 17 | Conglutin |
| 5D7 | Peanut | Ara h 2 | Site CR-A | 17 | Conglutin |
| 16G12 | Peanut | Ara h 2 | Site CR-A | 17 | Conglutin |
| [symbol] | Peanut | Are h 2 | Site CR-B | 17 | Conglutin |
| 11F10 | Peanut | Ara h 2 | Site CR-B | 17 | Conglutin |
| 15A4 | Peanut | Ara h 2 | Site CR-B | 17 | Conglutin |
| 2C9 | Peanut | Ara h 2 | Site CR-B | 17 | Conglutin |
| 15B8 | Peanut | Ara h 2 | Site CR-B | 17 | Conglutin |
| 26C3 | Peanut | Ara h 2 | Site CR-B | 17 | Conglutin |
| 7B6 | Peanut | Ara h 2 | Site CR-B | 17 | Conglutin |
| [symbol] | Peanut | Ara h 2 | Site SP-B | 17 | Conglutin |
| 9H11 | Peanut | Ara h 2 | Site CR-AB | 17 | Conglutin |
| 20G11 | Peanut | Ara h 2 | Site CR-C | 17 | Conglutin |
| [symbol] | Peanut | Ara h 3 | Site A | 57 | Glycinin |
| 15C4 | Peanut | Ara h 3 | Site A | 57 | Glycinin |
| 14G12 | Peanut | Ara h 3 | Site B | 57 | Glycinin |
| 17G11 | Peanut | Ara h 3 | Site B | 57 | Glycinin |
| 24C8 | Peanut | Ara h 3 | Site B | 57 | Glycinin |
| 17D7 | Peanut | Ara h 3 | Site B | 57 | Glycinin |
| 3E6 | Peanut | Ara h 3 | Site B | 57 | Glycinin |
| [symbol] | Peanut | Ara h 6 | Site SP-A | 15 | Conglutin |
| 22F2 | Peanut | Ara h 6 | Site SP-A | 15 | Conglutin |
| 15C2 | Peanut | Ara h 6 | Site SP-A | 15 | Conglutin |
| 26A5 | Peanut | Ara h 6 | Site SP-A | 15 | Conglutin |
| 6E10 | Peanut | Ara h 6 | Site SP-A | 15 | Conglutin |
| [symbol] | Peanut | Ara h 6 | Site SP-B | 15 | Conglutin |
| 1A8 | Peanut | Ara h 6 | Site SP-B | 15 | Conglutin |
| 20M10 | Peanut | Ara h 6 | Site SP-B | 15 | Conglutin |
| 9A11 | Peanut | Ara h 6 | Site SP-B | 15 | Conglutin |
| [symbol] | Peanut | Ara h 6 | Site SP-C | 15 | Conglutin |
| 3F9 | Peanut | Ara h 6 | Site SP-C | 15 | Conglutin |
| 3B7 | Peanut | Ara h 7 | Site A | 15 | Conglutin |
| 4D11 | Peanut | Ara h 7 | Site ? | 15 | Conglutin |
| 14C11 | Peanut | Ara h 7 | Site ? | 15 | Conglutin |
| 1C10 | Peanut | Ara h 9 | Site A | 10 | nsLTP |
| 31A12 | Peanut | Ara h ? | — | — | — |
| 7E6 | Peanut | Ara h ? | — | — | — |
| 3D2 | Peanut | Ara h ? | — | — | — |

All IgE mAbs were obtained from the peripheral blood cells of subjects known to have severe peanut allergy. MAb reactivity was determined using Phadia diagnostics and/or by ELISA and Western blot. nsLTP = non-specific lipid transfer protein. Antigenic sites were determined by competition ELISA. MAbs for which the inventor has made recombinant switched variant IgG are highlighted red. [symbol]
indicates data missing or illegible when filed

TABLE D
Genetic features of peanut allergen-specific human IgE mAbs

| | | | | | | | | Variable Gene | |
|---|---|---|---|---|---|---|---|---|---|
| | | | Germline Gene | | | | | Mutations | |
| IgE | | Light | Segments | | | AA Junction | CDR3 | VH | VH |
| mAb | Allergen | Chain | VH | D | JH | (SEQ ID NO:) | Length | NT | AA |
| 40C7 | Ara h 1 | K | 3-23 | 5-18 | 4 | CTKEGDTGSLWLFDSW (220) | 14 | 24 | 12 |
| 3B10 | Ara h 1 | K | 3-15 | 3-3 | 4 | CVTGNFDYRSSPLGFW (221) | 14 | 39 | 19 |
| 5C5 | Ara h 2 | K | 3-23 | 4-17 | 4 | CAKEDYDDRGFFDFW (222) | 13 | 28 | 15 |
| 32G9 | Ara h 2 | 2 | 1-8 | 2-15 | 4 | CARSQGTFEVYYFVSW (223) | 14 | 33 | 21 |
| 13D9 | Ara h 2 | 2 | 3-11 | 3-10 | 4 | CARRKFGAGSAIFDHW (224) | 14 | 29 | 14 |
| 9H11 | Ara h 2 | K | 3-30 | 1-26 | 4 | CAVGAPLEGYW (225) | 9 | 40 | 22 |
| 11F10 | Ara h 2 | K | 3-9 | 4-17 | 4 | CVKDNGLRTLDFW (226) | 11 | 10 | 9 |
| 15B8 | Ara h 2 | K | 3-30 | 3-3 | 4 | CARDANARFGVMIMAHW (227) | 15 | 47 | 22 |
| 5D7 | Ara h 2 | K | 7-4 | 2-8 | 6 | CARDFGERGNCVNGVCYGGYGMDVW | 23 | 11 | 6 |
| | | | | | | (228) | | | |
| 38B7 | Ara h 2 | K | 3-10 | 6-19 | 4 | CATTRSGWYFDYW (229) | 11 | 37 | 19 |
| 16G12 | Ara h 2 | 2 | 3-69 | 5-12 | 4 | CARDKGPIVPMPMDYW (230) | 14 | 44 | 19 |
| 26C3 | Ara h 2 | 2 | 4-4 | 2-2 | 2 | CARLRRVVPTAIWHFDLW (231) | 16 | 14 | 6 |
| 20G11 | Ara h 2 | K | 3-30 | 3-3 | 1 | CAKDGDYDSWSGLTEHFQHW (232) | 18 | 5 | 4 |
| 15A4 | Ara h 2 | K | 1-8 | 1-14 | 4 | CARGTDLNYW (233) | 8 | 27 | 15 |
| 7B6 | Ara h 2 | K | 4-34 | 3-10 | 1 | CARRRIGSLLKYFQDW (234) | 14 | 7 | 3 |
| 24C8 | Ara h 3 | K | 3-30 | 2-21 | 6 | CAKRTQKFAPYYFNGLDVW (235) | 17 | 24 | 11 |
| 3C3 | Ara h 3 | 2 | 4-39 | 3-9 | 2 | CARRKTYYDFLTDYYNWYFDVW | 20 | 42 | 20 |
| | | | | | | (236) | | | |
| 15C4 | Ara h 3 | 2 | 3-21 | 6-13 | 5 | CTRSYSSKYDNWFDPW (237) | 14 | 31 | 11 |
| 1H9 | Ara h 6 | K | 7-4 | 2-21 | 5 | CATDGSEGSW (238) | 8 | 22 | 10 |
| 8F3 | Ara h 6 | K | 4-39 | 3-16 | 3 | CARYVDYVWLRAFDIW (239) | 14 | 27 | 15 |
| 9A11 | Ara h 6 | K | 3-74 | 4-17 | 2 | CVRDRRAVTTARYFDLW (240) | 15 | 30 | 15 |
| 3F9 | Ara h 6 | 2 | 3-21 | 6-6 | 4 | CARAGAVRPGIGFHYFDNW (241) | 17 | 38 | 19 |
| 21C10 | Ara h 6 | 2 | 3-21 | 2-8 | 2 | CARKTNGAWFLDLW (242) | 12 | 27 | 14 |
| 22F2 | Ara h 6 | K | 7-4 | 1-26 | 5 | CSRDGSGASW (243) | 8 | 21 | 13 |
| 20M10 | Ara h 6 | 2 | 3-21 | 6-13 | 4 | CVRRGRGGAARALDNW (244) | 14 | 23 | 13 |
| 3B7 | Ara h 7 | K | 7-4 | 6-13 | 4 | CARGESLAALGSFAYW (245) | 14 | 7 | 6 |
| 4D11 | Ara h 7 | K | 3-11 | 3-16 | 3 | CARHLVRGTSLAAFDIW (246) | 15 | 20 | 12 |
| 14C11 | Ara h 7 | K | 4-39 | 2-2 | 6 | CARQRAALPPYYYYYFDVW (247) | 17 | 22 | 14 |

Antibody germline gene segment usages are shown for variable (V), diverse (D), and joining (J) regions of heavy chains based on the ImMunoGeneTics, IMGT database. The number of nucleotide and amino acid mutations are shown. As can be seen, all of the above antibody sequences are unique, arise from different germline gene segments, and are not clonally related.

TABLE 1
NUCLEOTIDE SEQUENCES FOR ANTIBODY VARIABLE REGIONS

| | SEQ ID | | |
|---|---|---|---|
| Clone | NO: | Chain | Variable Sequence Region |
| 40C7 | 1 | heavy | GAGGTGCAGCTGGTGGAGTCGGGGGGAGACTTGGTACAGCCGGGGGGGTCCCTGAGACT |
| | | | CTCTTGTGCAGCCTCTAGATTCAGCTTTGGCAGCTATGCCATGAGTTGGGTCCGCCAGGCTC |
| | | | CAGGGAAGGGCCTGGAGTGGGTCTCAGCGATTTCTTCTAGTGGTGGTAGGGCATACTACG |
| | | | CAGACTCCGTGAAGGGCCGGTTCACCATCTCCAGAGACAATTCCAGGAACACCCTGTATCT |
| | | | GCAGTTGAACAGCCTGAGAGGCGAGGACACGGCCTTCTATTACTGTACGAAAGAAGGTGA |
| | | | CACAGGGTCCCTATGGTTATTTGACTCCTGGGGCCAGGGAACCCTGGTCACCGTCTCCTCAG |
| | 2 | light | GACATCGTGATGACCCAGTCTCCAGACTCCCTGGCTGTGTCTCTGGGCGAGAGGGCCACCA |
| | | | TCAACTGCAAGTCCAGCCAGAGTGTTTTAGACAGCTCCAACAATAAGAAGTACTTAGCTTGG |
| | | | TACCAGCAGAAACCAGGACAGCCTCCTAAGCTGCTCATTTACTGGGCATCTACTCGGGAATT |
| | | | CGGGGTCCCTGACCGATTCAGTGGCAGCGGGTCTGGGACAGATTTCACTCTCACCATCAGC |
| | | | AGCCTGCAGGCTGAAGATGTGGCAGTTTATTACTGTCAGCAATATTATAGTACTCCTCCGAC |
| | | | GTTCGGCCAAGGGACCAAGGTGGAAATCAAAC |
| 3B10 | 3 | heavy | GAGGTGCAGCTGGTGGAGTCTGGGGGAGCCCTGGTAAAGCCGGGGGGGTCTCTTCGACTC |
| | | | TCCTGTGAAGGCTCTGGTTTCATGTTCAGTAGCGCCTGGTTGCACTGGGTCCGGCAGGCTCC |
| | | | AGGGATGGGACTGGAATTGGTCGGCCGTATGAAAAGTGAGACTGATGGTGGGACACTTGA |
| | | | CTACACTGCACCCGTGAGAGGCAGATTCACCATCTCAAGAGATGATTCAAAAAATATGTTTT |
| | | | TTCTGCAAATGAATAGCCTGAAAGCCGAGGACACCGCCGTCTATTATTGTGTCACAGGAAA |
| | | | CTTCGATTATAGGAGTTCACCCCTTGGCTTCTGGGGCCAGGGAGCCCTGGTCACCGTCTCCT |
| | | | CAG |
| | 4 | light | GACATCCAGATGACCCAGTCTCCTTCTACTCTGTCTGCGTCTGCAGGAGACAGAGTCACCAT |
| | | | CACTTGCCGGGCCAGTGAGAATATTTATAAGTGGTTGGCCTGGTATCAGCAGAAACCAGGG |
| | | | AAAGCCCCTAAATTGCTGATCTACAAGGTGTCTACTTTAGAAAGTGGGGTCCCATCACGGTT |
| | | | CAGCGGCAGTGGATTTGGGACAGAATTCACTCTCACCATCAGCAGCCTGCAGCCTGGTGAT |
| | | | TTTGCGACTTATTACTGCCAACATTATAACAGTTTCCCCTTCACTTTTGGCCAGGGGACCAAG |
| | | | CTGGAGGTCAAAC |
| 4G4 | 5 | heavy | GAGGTGCAGCTGGTGCAGTCGGGGGGAGGCTTGGTACAGCCTGGGGGGTCCCTGAGACT |
| | | | CTCCTGTGCAGCCTCTAGATTCATCTTTGGCAGCTATGCCATGAGTTGGGTCCGCCAGGCTC |
| | | | CAGGGAAGGGGCTGGAGTGGGTCTCAGCCATTAGTTCTAGTGGTGGCAGGACATACTACG |
| | | | CAGACTCCGTGAAGGGCCGGTTCACCATCTCCAGAGACAATTCCAGGAACACGCTGTACCT |
| | | | GCAGTTGAGCAGCCTGAGAGCCGAGGACACGGCCTTCTATTACTGTACGAAAGAAGGTGA |
| | | | CACAGGGTCCCTATGGCTTTTTGACTCCTGGGGCCAGGGAACCCTGGTCACCGTCTCCTCAG |
| | 6 | light | GACATCGTGATGACCCAGTCTCCAGACTCCCTGGCTGTGTCTCTGGGCGAGAGGGCCACCA |
| | | | TCAACTGCAAGTCCAGCCAGAGTGTTTTAGACAGCTCCAACAATAAGAAGTACTTAGCTTGG |
| | | | TACCAGCAGAAACCAGGACAGTCTCCTAAGCTGCTCATTTACTGGGCATCTACTCGGGAATT |
| | | | CGGGGTCCCTGACCGATTCAGTGGCAGCGGGTCTGGGACAGATTTCACTCTCACCATCAGC |
| | | | AGCCTGCAGGCTGAAGATGTGGCAGTTTATTACTGTCAGCAATATTATACTACTCCTCCGAC |
| | | | GTTCGGCCGAGGGACCAAGGTGGAAATCAAAC |
| 16G12 | 7 | heavy | GAGGTGCAGCTGGTGGAGTCTGGGGGAGGCTTAGTGAAGCCTGGAGGGTCCCTGAAACTC |
| | | | TCCTGTGCAGCCTCTGGATTCACTTTCAGTGACTATTACATGTATTGGGTTCGCCAGACTCCG |
| | | | GAAAAGAGGCTGGAGTGGGTCGCAACCATTAGTGATGGTGGTAGTTACACCTACTATCCAG |
| | | | ACAATGTAAAGGGCCGATTCACCATCTCCAGAGACAATGCCAAGAACACCCTGTACCTGCA |
| | | | AATGAGCCATCTGAAGTCTGAGGACACAGCCATGTATTACTGTGCAAGAGATAAAGGGCCT |
| | | | ATAGTCCCTATGCCTATGGACTACTGGGGTCAAGGAACCTCAGTCACCGTCTCCTCAG |
| | 8 | light | TCCTATGAGCTGACTCAGCCACTCTCAGTGTCCGTGTCCCCAGGACAGACTGTCAACATCAC |
| | | | CTGCTCTGGAGATAAATTGGGGGCCAAATTTACCTGCTGGTATCAGCAGAAGCCAGGCCAG |
| | | | TCCCCTATCCTAGTCCTTTATCAAGATACCAAGCGGCCCCCAGGGATCCCTGAGCGATTCTCT |
| | | | GGCTCCAACTCTGGGAACACAGCCACTCTGACCATCAGCGGGACCCAGGCTATGGATGAGG |
| | | | CTGACTATTACTGTCAGGCGTGGGAAAGTGGTATTGTGGCGTTCGGCGGAGGGACCAAGC |
| | | | TGACCGTCGTAG |
| 15B8 | 9 | heavy | CAGGTGCAGCTGGTGGAGTCTGGGGGACGCGTGGTCCAGCCGGGGGAGTCCCTGAGACTC |
| | | | TCCTGTGAAACATCTGGATTCCCCTTCCATGATCACGGCATGCACTGGGTCCGGCAGGCTCC |
| | | | AGGCAAGGGACTGGAGTGGGTGGCATTTATTCGATTTGATTCCACTTCCAAGTATACTTCAG |
| | | | ACTCCGTGAGGGGCCGATTCAGCATTTCCAGAGACAATTCAAAGAGCACACTATTTCTCCAA |
| | | | ATGAACAACCTGCGACGTGAGGACACGGCTATATATTACTGTGCGAGAGATGCCAACGCGA |
| | | | GATTTGGAGTAATGATCATGGCACATTGGGGCCAGGGAACCCTGGTCACCGTCTCCTCAG |
| | 10 | light | GACATCCAGTTGACCCAGTCTCCATCCTCCCTGTCTGCATCTGTCGGAGACAGAGTCACCAT |
| | | | CACTTGTCAGGCGAGTCAGGACATTGGACACTATTTAAATTGGTATCTGCAAAAACCAGGTC |
| | | | AAGCCCCGCAGGTCCTGATTTACGATGCATCCAATTTGGTAACAGGGGTCCCATCAAGATTC |
| | | | AGTGGAAGTGGATCTGGGACACATTTTACTTTCACCATCAGCAGCCTGCAGCCTGAAGATTT |
| | | | TGGAACATACTATTGTCATCAGCATGAGAAGCTTTACTCGATCTCCTTCGGCCAAGGGACAC |
| | | | GACTGGACATTAAAC |
| 15A7 | 11 | heavy | CAGGTGCAGCTGGTGCAGTCTGGGCCTGAAGTGAGGAAGCCTGGGGCCTCAGTGAAGGTC |
| | | | TCCTGTAAGACTTCTGGATACATGTTCACCGATTATGAAATGAATTGGGTGCGACAGGCCCC |
| | | | TGGACAGGGCCTTGAGTGGATGGGACTCATAAACCCGCACAGTGGTGACACAGGCTACGC |
| | | | ACAGAAGTTCCAGGGCAGAGTTAGCATGACCAGCGACACCTCCACAAGAACTTTCTACTTG |
| | | | GAGCTGACTGGCCTGACATCTGAGGACACGGCCGTGTATTACTGTGCGAGGAGCCAGGGT |
| | | | ACCTTCGAGGTTTACTACTTTGTCTCCTGGGGCCAGGGATCCCTAGTCACCGTCTCCTCAG |
| | 12 | light | TCCTATGTGCTGACTCAGCCACCCTCGGTGTCAGTGGCCCCAGGGAAGACGGCCAGCATTA |
| | | | CCTGTGGGGCAAAGAACATTGCAACAAAACGTGTACACTGGTACCGGCAGAAGCCAGGCC |
| | | | AGGCCCCTGTGGTGGTCGTCTCTGATGAGGAGGATCGATCCTCAGTGATCCCTGAGCGATT |
| | | | CTCTGGCTCCAAATCTGGGGACACGGCCACCCTGGAAATCAGCAGGGTCGAGGCCGGGGA |
| | | | TGAGGCCGATTATTACTGTCAGGTGTGGGATAGTCTGGCTGACCAAGTGGTATTCGGCGGA |
| | | | GGGACCAAGGTGACCGTCCTGG |
| 5D7 | 13 | heavy | CAGGTGCAGTTGGTGCAATCTGGGTCTGAGTTGAAGAAGCCTGGGGCCTCAGTGAAGGTT |
| | | | TCCTGCGAGGCCTCTGGATACAACCTCACTACCTATGCTATGAATTGGGTGCGACAGGCCCC |
| | | | TGGACAAGGGCTTGAGTGGATGGGATGGATCAACACCAACACTGGGAACCCAACGTATGC |
| | | | CCAGGACTTCACAGGACACTTTGTCTTCTCCCTGGACACCTCTGTCAGTACGGCATATCTGC |
| | | | AGATCAGCAGCCTAAAGGCTGAGGACACTGCCGTGTATTACTGTGCGAGAGATTTCGGGG |
| | | | AGCGGGGAAATTGTGTTAATGGTGTATGCTATGGGGGTTACGGTATGGACGTCTGGGGCC |
| | | | AAGGGACCACGGTCACCGTCTCCTCA |
| | 14 | light | GACATCCAGATGACCCAGTCTCCTTCCACCCTGTCTGCATCTGTAGGAGACAGAGTCACCAT |
| | | | CACTTGCCGGGCCAGTCAGAGTATTAGCCGGTGGTTGGCCTGGTATCAGCAGAAACCAGG |
| | | | GAAAGTCCCTAAGCTCCTGATCTATAAGGCGTCTAGATTAGAAGGTGGGGTCCCATCAAGG |
| | | | TTCAGCGGCAGTGGATCTGGGACAGAATTCACTCTCACCATCAGCAGCCTGCAGCCTGATG |
| | | | ATTTTGCAACTTATTACTGCCAAGAATATACTAATTATTCGGGGACGTTCGGCCAAGGGACC |
| | | | AAGGTGGAAATCAGAC |
| 14B3 | 15 | heavy | CAGGTGCAGCTGGTGCAGTCTGGGCCTGAAGTGAAGAAGCCTGGGGCCTCAGTGAAGGTC |
| | | | TCCTGTAAGACTTCTGGATACACGTTCACCGACTCTGAAATGAACTGGGTGCGACAGGCCCC |
| | | | TGGACAGGGCCTTGAGTGGATGGGACTCATAAATCCGCACAGTGGTGACACACGCTATGCA |
| | | | GAGAGGTTCCAGGGCAGAGTCACCATGACCAGCGACACCTCCATAAACACAGTCTACTTGG |
| | | | AGTTGAGTGGCCTGACATCTGAGGACACGGCCATATATTTCTGTGCGAGGAGTCAGGGGA |
| | | | CCTTCGAGGTTTACTACTTTCTCTCCTGGGGCCAGGGATCGCTAATCACCGTCTCCTCAG |
| | 16 | light | |
| 2C9 | 17 | heavy | CAGGTGCAGCTGGTGGAGTCTGGGGGAGGCTTGGTCAAGCCTGGAGGGTCCCTGAGACTC |
| | | | TCCTGTGCAGCCTCTGGATTCAGCTTCAGTGACTTCTACATGAGCTGGATACGCCAGGCTCC |
| | | | AGGCAAGGGACTGGAGTGTATTTCTTATATGAGTAGTAGTGGTGGTAACATATACTATGCA |
| | | | GACTCTGTGAAGGGCCGATTCACCATCTCCAGGGACAACGCCAAGAATTCACTGTCTCTGCA |
| | | | AATGAACAGCCTGAGAGCCGACGACACGGCCCTCTATTATTGTGCGAGACGGAGGTATGGT |
| | | | TCAGGAAGTTCGATCTTTGACTACTGGAGCCAGGGAACCCTGGTCACCGTCTCCTCAG |
| | 18 | light | TCCTATGAGCTGACTCAGCCACCCTCAGTGTCCGTGTCCCCAGGACAGACAGCCAGCGTCAC |
| | | | CTGCTCAGGAGATACATTGGGTGATAGATATGTGAGTTGGTATCAGCAGAAGGCAGGCCA |
| | | | GTCCCCTGTCTTGGTCATCTATCAAAGTGGCCAGCGGCCCTCAGGGATCCCTGAGCGATTCT |
| | | | CTGGCTCCAACTCTGGGAACACAGCCACTCTGAGTATCAGCGAGACCCAGGCGCTGGATGA |
| | | | GGCTGACTATTATTGTCTGACGTGGGACCGCGGCACTCCTGTCTTCGGACCTGGGACCACA |
| | | | GTCACCGTCGTAG |
| 2F12 | 19 | heavy | CAGGTGCAGCTGGTGCAGTCTGGGCCTGAAGTGAAGAAGCCTGGGGCCTCAGTGAAGGTC |
| | | | TCCTGTCAGACTTCTGGATATACCTTCACCGATTATGAAATGAACTGGGTGCGACAGGCCCC |
| | | | TGGACAGGGCCTTGAGTGGATGGGATTAATAAACCCGCACAGTGGTGACACAGGCTATGC |
| | | | ACTGAAGTTCCAGGGCAGAGTCACCATGACCAGCGACACCTCCAAAAGCACAGTGTACTTG |
| | | | GAGTTGAGTGGCCTGACATCTGAAGACACGGCCGTATATTACTGTGCGAGGAGTCAGGGA |
| | | | ACCTTCGAGGTTTACTACTTTGTCTCCTGGGGCCAGGGATCCCTAGTCACCGTCTCCTCAG |
| | 20 | light | TCCTATGTTCTGACTCAGCCACCTTCGGTGTCAGTGGCCCCAGGGAAGACGGCCACCATTAC |
| | | | GTGTGGGGCTAACGGAATTGGCAGGAAACGTGTGCACTGGTATAGCCAGAGGCCAGGCCA |
| | | | GGCCCCTGTGGTGGTCGTTTCTGATGATGACGATCGATCCTCAGTGAACCCTGAACGATTCT |
| | | | CTGCCTCCAAATCTGGGGACACGGCCGCCCTGACAATCACCAGGGTCGAGGCGGGGGATG |
| | | | AGGCCGATTATTACTGTCAGGTGTGGGATAGTAGGACTGACCAAGTGGTGTTCGGCGGAG |
| | | | GGACCAAGGTGACCGTCCTGG |
| 29B3 | 21 | heavy | CAGGTGCAGCTGGTGCAGTCTGGGCCTGAAGTGAAGAAGCCTGGGGCCTCAGTGAAGGTC |
| | | | TCCTGTAAGACTTCTGGATACAGCTTCACCGATTATGAAATGAACTGGGTGCGACAGGCCCC |
| | | | TGGACAGGGCCTTGAGTGGATTGGATTGGTAAACCCGCACAGTGGTGAGACAGGCTATGC |
| | | | ACTGAAGTTCCAGGGCAGAGTCACCATGACCAGCGACACCGCCATAAGTACAGTATACTTG |
| | | | GAGTTGAGTGGCCTGACAACTGAGGACACGGCCATATATTATTGTGCGAGGAGTCAGGGA |
| | | | ACCTTCGAGGTTTATTACTTTGTCTCCTGGGGCCAGGGATCCCTAGTCACCGTCTCCTCAG |
| | 22 | light | |
| 38B7 | 23 | heavy | CAGGTGCAGCTGGTGGAGTCGGGGGGAGGCGTGGTCCAGCCTGGGAGGTCCCTGAGACT |
| | | | CTCCTGTGTAGGTTCTGGATTCACATTCAGAAGGGATGCTAAGTACTGGGTCCGCCAGGCT |
| | | | CCAGGTAAGGGGCTAGAATGGGTGGCAGCGATTTCGCATGATGGCGGTGAGGAAGACTAC |
| | | | GCAGAGTCCGTGAAGGGCCGATTCACCATTTCCAGAGACAATTCCAGGGAAACAGTTTATC |
| | | | TGGAAATGAACAGCCTGAGACCTGAAGACACGGCTGTCTATTATTGTGCGACTACTCGGAG |
| | | | TGGCTGGTACTTTGACTACTGGGGTCAGGGAGCCCTGGTCACCGTCTCCTCAG |
| | 24 | light | GAAATTGTACTGACGCAGTCTCCAGCCACCCTGTCTTTGTCTCCAGGGGCTAGAGCCACCCT |
| | | | CTCCTGCAGGGCCAGTCAGAGTGTTGACACCTACTTAGCCTGGTACCAACAGAGACGTGGC |
| | | | CAGTCTCCCAGGCTCCTCATCTATGATGCATCCAAGAGGGCCACTGGCATCCCAGCCAGGTT |
| | | | CAGTGGCAGTGGGTCTGGGACAGATTTCACTCTCACCATCAGCAGCCTAGAGCCTGAAGAT |
| | | | TTTGCAGTTTATTACTGTCAGCAGCGTAACAACTGGCGGACGTGGACGTTCGGCCAAGGGA |
| | | | CCAAGGTGGAAATCAACC |
| 16A8 | 25 | heavy | CAGGTGCAGCTGGTGCAGTCTGGGCCTGACGTGAGGAAGCCTGGGGCCACAGTGAAGGTC |
| | | | TCCTGTCAGACCTCTGGATATAGGTTCACCGATTATGAAATGAACTGGGTGCGACAGGCCC |
| | | | CTGGACAGGGCCTTGAGTGGATAGGGTTGATAAATCCGCACAGTGGTGACACAGCATATG |
| | | | CACAGAAATTCCAGGGCAGAGTCACCATGACCAGCGACACATCCGATAGCACAGTCTATTT |
| | | | GGAACTGAGTGGCCTGACACCTGATGACACGGCCGTGTACTACTGTGCGAGGAGCCAGGG |
| | | | TACCTTCGAGGTTTACTACTTTGTCTCCTGGGGCCAGGGATCCCTAGTCACCGTCTCCTCAG |
| | 26 | light | TCCTATGTGTTGACTCAGCCTCCCTCGATGTCAGTGGCCCCAGGGACGACGGCCAGCATTCC |
| | | | CTGTGGGGCAAACAACATTGCCAAGAAACGTGTTCACTGGTACCGCCAGAAGCCAGGCCA |
| | | | GGCCCCTGTGGTGGTCGTCTCTGATGATGAGGATCGATCCTCAGTGATCCCTGACCGATTCT |
| | | | CTGGCTCCAAATCTGGGGACACGGCCACCCTGACAATCAGCAGGGTCGAGGCCGGGGATG |
| | | | AGGGCGACTATTATTGTCAGGTGTGGGATAGTAAGACTGACCACGTGGTTTTCGGCGGAG |
| | | | GGACCAAGGTGACCGTCCTGG |
| 11F10 | 27 | heavy | GAAGTGCAGCTGGTGGAGTCTGGGGGAGGCTTGGTACAGCCTGGCAGGTCCCTGAGACTC |
| | | | TCCTGTGCAGCCTCTGGATTCACCTTTGATGATTATACCATGCACTGGGTCCGGCAAGCTCC |
| | | | AGGGAAGGGCCTGGAGTGGGTCTCAAGTATTCGTTGGAATAGTGGTAACTTAGACTATGC |
| | | | GGACTCTGTGAAGGGCCGATTCACCATCTCCAGAGACAACGCCAGGCACTCCCTGTATCTG |
| | | | CAAATGAACAGTCTGAGAGCTGAGGACACGGCCTTATATTACTGTGTAAAAGATAACGGCC |
| | | | TACGGACTCTAGACTTCTGGGGCCAGGGAACCCTGGTCACCGTCTCCTCAG |
| | 28 | light | GATATTGTGATGACTCAGTCTCCACTCTCCCTGCCCGTCACCCCTGGAGAGCCGGCCTCCAT |
| | | | CTCCTGCAGGTCTAGTCAGAGCCTCCTCCATAGGAATGGATACAACTATTTGGATTGGTACC |
| | | | TGCAGAAGCCAGGGCAGTCTCCACAGCTCCTGATCTCTTTGGCTTCTAATCGGGCCTCCGGG |
| | | | GTCCCTGACAGGTTCAGTGGCAGTGGATCAGGCACAGATTTTACACTGAAAATCAGCAGAG |
| | | | TGGAGGCTGAGGATGTTGGGGTTTATTACTGCATGCAAGCTCTACAAACTTGGACGTTCGG |
| | | | CCAGGGGACCAAGGTGGAACTCAAAC |
| 32G9 | 29 | heavy | CAGGTGCAGCTGGTGCAGTCTGGGCCTGAAGTGAGGAAGCCTGGGGCCTCAGTGAGGGTC |
| | | | TCCTGTACGACTTCTGGTTACACATTCATCGATTATGAAATGAACTGGGTGCGACAGGCCCC |
| | | | TGGACAGGGCCTTGAGTGGATGGGATTAATAAACCCGCACAGTGGTGACACAGGCTATGC |
| | | | ACAGAAGTTCCAGGACAGAGTCACCATGACCAGCGACACCTCCTTAAGCACAGCCTACTTG |
| | | | GAGTTGAGTGGCCTGACATCTGAGGACACGGCCGTATATTACTGTGCGAGGAGTCAGGGT |
| | | | ACCTTCGAGGTCTACTACTTTGTCTCCTGGGGCCAGGGATCCCTAGTCACCGTCTCCTCAG |
| | 30 | light | TCCTATGTGCTGACTCAGCCACCCTCGGTGTCAGTGGCCCCAGGAAAGACGGCCAGCATAA |
| | | | CCTGTGAGGCAAAAAACATTGTAAGAAAACGTGTGCATTGGTACCGCCAGAAGCCAGGCC |
| | | | AGGCCCCGGTGGTGGTCGTCTCTGATGATGAGGATCGATCCTCAGTGATCCCTGAGCGAGT |
| | | | CTCTGGCTCCAAATCGGGGGACACGGCCACCCTGACAATCAGCAGGGTCGAGGCCGGGGA |
| | | | TGAGGCCGATTATTACTGTCAGGTGTGGGATAGTACGACTGACCAGGTGGTATTCGGCGGA |
| | | | GGGACCAAGGTGACCGTCCTGG |
| 24F10 | 31 | heavy | CAGGTGCAGCTGGTGCAGTCTGGGCCTGAAGTGAAGAAGCCTGGGGCCTCAGTGAAGGTC |
| | | | TCCTGTCAGACTTCTGGATATACCTTCACCGATTATGAAATGAACTGGGTGCGACAGGCCCC |
| | | | TGGACAGGGCCTTGAGTGGATGGGATTAATAAACCCGCACAGTGGTGACACAGGCTATGC |
| | | | ACTGAAGTTCCAGGGCAGAGTCACCATGACCAGCGACACCTCCAAAAGCACAGTGTACTTG |
| | | | GAGTTGAGTGGCCTGACATCTGAAGACACGGCCGTATATTACTGTGCGAGGAGTCAGGGA |
| | | | ACCTTCGAGGTTTACTACTTTGTCTCCTGGGGCCAGGGATCCCTAGTCACCGTCTCCTCAG |
| | 32 | light | |
| 15A4 | 33 | heavy | |
| | 34 | light | GACATCGTCCTGACCCAGTCTCCAGACTCCCTGGCTGTGTCTCTGGGCGAGAGGGCCACCA |
| | | | TCAACTGCAAGTCCAGCCAGAATATTTTAGACAACTCCAACAATAAGAACTTCATAGCTTGG |
| | | | CACCAGCATAAACCAGGACAGCCTCCTAAACTGCTCATTTACTGGGGTTCTTCCCGGGAATC |
| | | | CGGGGTCCCTGACCGATTCAGTGGCAGCGGGTCTGGGACAGATTTCACTCTCACCATCAGC |
| | | | AACCTGCAGGCTGAAGATGTGGCAGTTTATTACTGTCTCCAATATTATAGTCTTCCTCACACT |
| | | | TTTGGTCAGGGGACCAAGGTGGAGATCAAAC |
| 13D9 | 35 | heavy | CAGGTGCAGCTGGTGGAGTCTGGGGGAGGCTTGGTCAAGCCTGGAGGGTCCCTCAGACTC |
| | | | TCCTGCGTAGCCTCTGGATTCACCTTCAGTGACTTCTACATGAGCTGGATCCGCCAGGCTCC |
| | | | AGGGAAGGGCCTTGAGTGGGTGTCCTACATGAGTGCAACTGGCGGTAATATATACTATGCA |
| | | | GACTCTATGAAGGGCCGATTAACTATCTCCAGGGACAACACCAAGAACTCATTGTTTCTCCA |
| | | | AATGAACAGCCTGAGAGCCGACGACACGGCCCTGTATTATTGTGCGAGGCGGAAGTTTGGT |
| | | | GCAGGGAGTGCGATCTTTGACCACTGGAGCCAGGGAACCCTGGTCACCGTCTCCTCAG |
| | 36 | light | TCCTATGAACTGACTCAACCACCCTCAGTGTCCGTGTCCCCAGGACAGACAGCCAGCGTCAC |
| | | | CTGCTCTGGAGACAAATTGGGTGAAAGATATGTGAGTTGGTATCAGCAGAAGGCAGGCCA |
| | | | GTCCCCTGACTTGGTCATCTATCAAACTAACCAGCGGCCCTCAGGGATCCCTGAGCGATTCT |
| | | | CTGGCTCCGACTCTGGGAACACAGCCACTCTGACTATCAGCGGGACCCAGGGTCTGGATGA |
| | | | GGCAGACTATTACTGTCTGACGTGGGACCGCGGCACTCCTGTCTTCGGAACTGGGACCAAA |
| | | | GTCACCGTCCTAG |
| 5C5 | 37 | heavy | GAGGTGCAGTTGTTGGAGTCAGGGGGAGGCTTGGTACAGCCGGGGGGGTCCCTGAGACT |
| | | | CTCCTGTGCAGCCTCTGGATTCACCTTTAGCAACCATGCCATGAGCTGGGTCCGCCAGACTC |
| | | | CAGGGGAGGGGCTGCAGTGGGTCTCAGCTCTTACTTATAGTGGTAAGACCACATACTACGC |
| | | | AGACTCCGTGAAGGGCCGGTTCACCATCTCCAGAGACAATTCCAAGAATTTACTATTTCTGC |
| | | | AAATGAACAGCCTGAGAGCCGGGGACACGGCCATATATTACTGTGCGAAGGAGGACTACG |
| | | | ATGACCGGGGCTTCTTTGACTTCTGGGGCCAAGGGACAAGGGTCACCGTCTCCTCAG |
| | 38 | light | GACATCCAGATGACCCAGTCTCCATCCTCCCTGTCTGCGTCTGTGGGAGACAGAGTCACCAT |
| | | | CACTTGCCGGGCAAGTCAGACCATTAGTACTTATTTACATTGGTATCAACAAAAACCAGGCA |
| | | | AAGCCCCTAACCTCCTCATCTATGCTGCATCCACTTTGCAAAGTGGGGTCCCATCAAGGTTC |
| | | | AGTGGCAGTGGATCTGGGACAGATTTCAGTCTCACCATCAGTAGTCTGCGTCCTGAAGATTT |
| | | | TGCAATTTACTACTGTCAACAGGGTTACAATAACCCGTACACTTTTGGCCAGGGGACCAAAG |
| | | | TGGATATCAAAA |
| 14G12 | 39 | heavy | CAGGTGCAGCTGGTGGAGTCTGGGGGAGGCGTGGTCCAGCCTGGGAGGTCGCTGCGACTC |
| | | | TCCTGTGCAGGCTCTGGATTCAGGTTCAGTGACTATGGCATGCACTGGGTCCGCCAGGCTC |
| | | | CAGGCAAGGGGCTGGAGTGGGTGGCTATAGTTGCATATGATGAGAGCAAGAAATACTATG |
| | | | CAGACTCCGTAAAGGGCCGATTCACCATCTCCAGAGACAATTCCAAGAACACGCTGTTTCTG |
| | | | CAAATGAACAGCCTGAGAGCTGACGACACGGCTGTCTATTACTGTGCGAAACGGACCCAAA |
| | | | AATTTGCCCCCTATTATTTCAACGGTTTGGATGTCTGGGGCCAAGGGACCACGGTCACCGTC |
| | | | TCCTCA |
| | 40 | light | GATATTGTGATGACTCAGTCTCCACTCTCCCTGCCCGTCACCCCTGGAGAGCCGGCCTCCAT |
| | | | CTCCTGTAGGTCTAGCCAGAGCCTCCTGGATCGTAATGGATACAACTACTTGGATTGGTACG |
| | | | TGCAGAAGCCAGGGCAGTCTCCACAGCTCCTGATGTATTTGGGTTCTGATCGGGCCTCCGG |
| | | | GGTCCCTGCCAGGTTCAGTGGCAGTGGATCAGGCACAGATTTTACACTGAAAATCAGTAGA |
| | | | GTGGAGGTTGAGGATGTTGGGGTTTATTACTGCATGCAAGCTCTACAAATTCCGATCACCTT |
| | | | CGGCCAAGGGACACGACTGGAGATTAAAC |
| 17D7 | 41 | heavy | CAGGTGCAGCTGGTGGAGTCTGGGGGAGGCGTGGTCCAGCCTGGGAGGTCGCTGCGACTC |
| | | | TCCTGTGCAGGCTCTGGATTCAGGTTCAGTGACTATGGCATGCACTGGGTCCGCCAGGCTC |
| | | | CAGGCAAGGGGCTGGAGTGGGTGGCTATAGTTGCATATGATGAGAGCAAGAAATACTATG |
| | | | CAGACTCCGTAAAGGGCCGATTCACCATCTCCAGAGACAATTCCAAGAACACGCTGTTTCTG |
| | | | CAAATGAACAGCCTGAGAGCTGACGACACGGCTGTCTATTACTGTGCGAAACGGACCCAAA |
| | | | AATTTGCCCCCTATTATTTCAACGGTTTGGATGTCTGGGGCCAAGGGACCACGGTCACCGTC |
| | | | TCCTCA |
| | 42 | light | GATATTGTGATGACTCAGTCTCCACTCTCCCTGCCCGTCACCCCTGGAGAGCCGGCCTCCAT |
| | | | CTCCTGTAGGTCTAGCCAGAGCCTCCTGGATCGTAATGGATACAACTACTTGGATTGGTACG |
| | | | TGCAGAAGCCAGGGCAGTCTCCACAGCTCCTGATGTATTTGGGTTCTGATCGGGCCTCCGG |
| | | | GGTCCCTGCCAGGTTCAGTGGCAGTGGATCAGGCACAGATTTTACACTGAAAATCAGTAGA |
| | | | GTGGAGGTTGAGGATGTTGGGGTTTATTACTGCATGCAAGCTCTACAAATTCCGATCACCTT |
| | | | CGGCCAAGGGACACGACTGGAGATTAAAC |
| 3C3 | 43 | heavy | CAGCTGCAGCTCCAGGAGTCGGGCCCAGGACTAGTTAAGCCTTCGGAGACCCTGTCCCTGA |
| | | | CCTGCTCCATTTCTGGTGCCTCCATCAACAGTGATGATTATTATTGGGGCTGGATTCGGCAG |
| | | | GCCCCAGGGGAGGCACTGGAGTGGATTGCGAGTATCGATGCTAGTGGGACCACGTTCTAC |
| | | | AATCCGTCCCTCAGAAGTCGGGTCACCATCTCCGTGGACACGTCCACGAACCAAATCTCCCT |
| | | | GAGGCTGAACTCTGTGACCGCCGCAGACACGGCTATATATTACTGTGCGAGACGGAAGACC |
| | | | TATTACGATTTTTTGACTGATTATTACAATTGGTACTTCGATGTCTGGGGCCGTGGCACCCTG |
| | | | GTCACCGTCTCCTCAG |
| | 44 | light | CAGTCTGCCCTGACTCAGCCTCGCTCAGTGTCCGGGTCTCCTGGGCAGTCAGTCACCTTCTC |
| | | | CTGCACTGGAATTAATAGTGACATTCGTGATTATGATTATGTCTCATGGTATCAACAACACC |
| | | | CAGGCGAGGCCCCCAAACTCATCATTTATGATGTCACTAAACGGGCCTCAGGGGTCCCTTCT |
| | | | CGCTTCTCTGGCTCCAAGTCTGGCAACACGGCCTCCCTGACCATCTCTGGGCTCCAGGCTGA |
| | | | AGATGAGGCGGACTATTACTGCTGTTCGTATTCAGTCACTTACTCTTTCGAGGTCTTTGGAA |
| | | | CTGGGACCCAGGTCTCCGTCCTAG |
| 17G11 | 45 | heavy | CAGGTGCAGCTGGTGGAGTCTGGGGGAGGCGTGGTCCAGCCTGGGAGGTCGCTGCGACTC |
| | | | TCCTGTGCAGGCTCTGGATTCAGGTTCAGTGACTATGGCATGCACTGGGTCCGCCAGGCTC |
| | | | CAGGCAAGGGGCTGGAGTGGGTGGCTATAGTTGCATATGATGAGAGCAAGAAATACTATG |
| | | | CAGACTCCGTAAAGGGCCGATTCACCATCTCCAGAGACAATTCCAAGAACACGCTGTTTCTG |
| | | | CAAATGAACAGCCTGAGAGCTGACGACACGGCTGTCTATTACTGTGCGAAACGGACCCAAA |
| | | | AATTTGCCCCCTATTATTTCAACGGTTTGGATGTCTGGGGCCAAGGGACCACGGTCACCGTC |
| | | | TCCTCA |
| | 46 | light | GATATTGTGATGACTCAGTCTCCACTCTCCCTGCCCGTCACCCCTGGAGAGCCGGCCTCCAT |
| | | | CTCCTGTAGGTCTAGCCAGAGCCTCCTGGATCGTAATGGATACAACTACTTGGATTGGTACG |
| | | | TGCAGAAGCCAGGGCAGTCTCCACAGCTCCTGATGTATTTGGGTTCTGATCGGGCCTCCGG |
| | | | GGTCCCTGCCAGGTTCAGTGGCAGTGGATCAGGCACAGATTTTACACTGAAAATCAGTAGA |
| | | | GTGGAGGTTGAGGATGTTGGGGTTTATTACTGCATGCAAGCTCTACAAATTCCGATCACCTT |
| | | | CGGCCAAGGGACACGACTGGAGATTAAAC |
| 15C4 | 47 | heavy | GAGGTGCAGCTGGTGGAGTCGGGGGGAGGCCTGGTCAAGCCTGGGGGGTCCCTGAGACT |
| | | | CTCCTGTGCAGCCTCTGGATTCACGTTCAGTCTGTACACCATGAACTGGGTCCGCCAGGCTC |
| | | | CAGGGAAGGGGCTGGAGTGGGTCTCATCGATTACGTCAAATAGTAGTCACTTGTACTATGT |
| | | | TGATTCCGTGAAGGGCCGGTTCACCATCTCCAGAGACAACTCCAAGAACTCAGTGTCTCTGC |
| | | | AAATGAACAGCCTGAGAGCCGAGGACACGGCTGTGTATTATTGTACGAGGTCTTACAGCAG |
| | | | CAAATATGACAACTGGTTCGACCCCTGGGGCCAGGGGATGCTGGTCACCGTCTCCTCAG |
| | 48 | light | TCCTATGAGCTGACTCAGCCACCCTCCGTGTCCGTGTCCCCAGGACAGACAGCCAGCATCAT |
| | | | GTGCTCTGGAGATAACTTGGGGGATAAGTATGTTTGCTGGTATCAACAGAAGCCAGGCCAC |
| | | | TCCCCTGAATTGGTCATGTATCGAGATAATAAGAGGCCCTCAGGGGTCCCTGAGCGATTCTC |
| | | | TGGCTCCAAGTCTGGGAACACTGCCACTCTGACCATCAGCGGGTCCCAGGCCATGGATGAG |
| | | | GCTGACTATTACTGTCAGGCGTTGGACAGTGGCAGTTTTTGGGTGTTCGGCGGAGGGACCA |
| | | | AGCTGACCGTCCTGG |
| 24C8 | 49 | heavy | CAGGTGCAGCTGGTGGAGTCTGGGGGAGGCGTGGTCCAGCCTGGGAGGTCGCTGCGACTC |
| | | | TCCTGTGCAGGCTCTGGATTCAGGTTCAGTGACTATGGCATGCACTGGGTCCGCCAGGCTC |
| | | | CAGGCAAGGGGCTGGAGTGGGTGGCTATAGTTGCATATGATGAGAGCAAGAAATACTATG |
| | | | CAGACTCCGTAAAGGGCCGATTCACCATCTCCAGAGACAATTCCAAGAACACGCTGTTTCTG |
| | | | CAAATGAACAGCCTGAGAGCTGACGACACGGCTGTCTATTACTGTGCGAAACGGACCCAAA |
| | | | AATTTGCCCCCTATTATTTCAACGGTTTGGATGTCTGGGGCTAAGGGACCACGGTCACCGTC |
| | | | TCCTCAG |
| | 50 | light | GATATTGTGATGACTCAGTCTCCACTCTCCCTGCCCGTCACCCCTGGAGAGCCGGCCTCCAT |
| | | | CTCCTGTAGGTCTAGCCAGAGCCTCCTGGATCGTAATGGATACAACTACTTGGATTGGTACG |
| | | | TGCAGAAGCCAGGGCAGTCTCCACAGCTCCTGATGTATTTGGGTTCTGATCGGGCCTCCGG |
| | | | GGTCCCTGCCAGGTTCAGTGGCAGTGGATCAGGCACAGATTTTACACTGAAAATCAGTAGA |
| | | | GTGGAGGTTGAGGATGTTGGGGTTTATTACTGCATGCAAGCTCTACAAATTCCGATCACCTT |
| | | | CGGCCAAGGGACACGACTGGAGATTAAAC |
| 1A8 | 51 | heavy | CAGCTGCAGCTGCAGGAGTCGGGCCCAGGACTGGTGAAGCCTTCGGAGACCCTGTCACTCA |
| | | | CCTGCAGTGTCTCTGATGACTCCATCAGTACTCCTAGTTACTTCTGGACCTGGATCCGCCAGC |
| | | | CCCCAGGGAAGGGGCTGGAGTGGATAGCCAGTATCTATTATACTGGGACCACCTACTACAA |
| | | | CCCGTCCCTCAAGAGTCGAGTCACCTTATCCGTCGACACGCCCAAGAGGCAGTTCTTCCTGA |
| | | | GGCTGAGCTCTGTGACCGCCGCAGACACGGCTGTTTATTACTGTGCGAGATATCTTGATTAC |
| | | | GTTTGGTTGAGGGCTTTTGATGTCTGGGGCCAAGGGGCAATGGTCACCGTCTCTTCAG |
| | 52 | light | GAAATTGTGTTGACACAGTCTCCAGCCACCCTGTCTTTGTCTCCAGGGGAAAGAGCCACCCT |
| | | | CTCCTGCAGGGCCAGTCCGAGTGTTGGCAGGTTCTTAGCCTGGTACCAGCAGAAACCTGGC |
| | | | CAGGCTCCCAGGCTCCTCATCTATGATGCATCTCAGAGGGCCACTGACATCCCAGCCAGGTT |
| | | | CAGTGCCAGTGGGTCTGGGACAGACTTCACTCTCACCATCGACAGCCTAGAGCCTGAAGAT |
| | | | TTTGCAATATATTACTGTCAGCACCGTAGCAACTGGCCGGTCACTTTCGGTGGAGGGACCA |
| | | | GGGTGGAGATCAAGC |
| 3F9 | 53 | heavy | GAGGTGCAGCTGGTGGAGTCTGGGGGAGGCCTGGTCAAGCCTGGGGGGTCCCTGAGGCT |
| | | | CTCCTGTATAGGCTCTGGATTCGACTTCAGTAGAGATACTTTCCACTGGGTCCGCCAGGCTC |
| | | | CCGGGAAGGGGCTGGAGTGGATTTCAAGCATCAGTCGTATTGAGACTTACACATACTACGT |
| | | | CGACTCAGTGAAGGGCCGATTCACCGTCTCCAGAGACAACGCCAAGAATTCAGTCTATCTA |
| | | | CAAATGAACAGCCTGAGAGTCGAAGACACGGCTGTCTATTTTTGTGCGAGAGCAGGAGCA |
| | | | GTTCGTCCCGGAATTGGATTTCACTACTTTGACAATTGGGGCCAGGGAAGCCCGGTCACCG |
| | | | TCTCCTCAG |
| | 54 | light | |
| 6E10 | 55 | heavy | |
| | 56 | light | GACATCCAGATGACCCAGTCTCCTTCCACCGTGTCTGCATCTGTAGGAGACAGAGTCACCAT |
| | | | CACTTGTCGGGCCAGTCAGAGCATTGGTCGCTGGTTGGCCTGGCATCAGCAGAAGCCAGG |
| | | | GAAAGCCCCGAAAGTCCTGATTACTAGGGCCTCTAATGTAGAAAGTGGGGTCCCATCAAGG |
| | | | TTCAGCGGCAGTGGATCTGGGACAGAATTCACTCTCACCATCAGCAGCCTGCAGCCTGATG |
| | | | ATTTTGCAACTTATTACTGTCAACAATATAATACTAATTCGGGGACATCCGGCCAAGGGACC |
| | | | AAGGCGGAAATCAAAC |
| 4D11 | 57 | heavy | CAGGTGCAGCTGGTGGAGTCTGGGGGAGGCTTGGTCAAGCCTGGAGGGTCCCTGAGACTC |
| | | | TCCTGTACAGCCTCTGGATTCAACTTTACTAACTACTACATGAGCTGGATCCGCCAGGCTCCA |
| | | | GGGAAGGGCCTGGAGTGGGTTTCATACATTAGTAGTACTACTAATAGCATAGATTACGCAG |
| | | | ACTCTGTGAAGGGCCGATTCACCATCTCCAGGGACAACGCCAAGAAGTCACTGTATCTGCA |
| | | | GATGAACAGCCTAAGAGCCGACGACACGGCCTTCTATTACTGTGCGCGACATTTGGTCAGG |
| | | | GGGACCTCTCTTGCTGCTTTTGATATCTGGGGCCAAGGGACAATGGTCACCGTCTCCTCAG |
| | 58 | light | |
| 1H9 | 59 | heavy | CAGGTGCAGCTGGTGCAATCTGGGTCTGAGTTGAGGAAGCCTGGGGCCTCAGTGAAGGTT |
| | | | TCCTGCAAGGCTTCCGGATACACCTTCACTAAGTATGGTATGAATTGGGTGCGACAGGCCC |
| | | | CTGGACAAGGACTGGAGTGGATGGGATGGATTAACACGAACACTGCAAAGCCAACGTATG |
| | | | CCCAGGACTTCACAGGACGATTTGTCTTCTCTTTGGACACCTCTGTCAACACGGCATATCTG |
| | | | GAGATCAGCGGCCTAAAGGCTGAAGACACCGCCGTCTATTACTGTGCGACAGATGGTAGTG |
| | | | AGGGCTCCTGGGGCCAGGGAACCCTGGTCACCGTCTCCTCA |
| | 60 | light | GACATCCAGATGACCCAGTCTCCTTCCACCCTGTCTGCATCTGTAGGAGACAGAGTCACCAT |
| | | | CACTTGCCGGGCCAGTCAGAGTATTGGTACCTGGTTGGCCTGGCATCAGCAGAAACCAGGG |
| | | | ACAGCCCCTAAGGTCCTGATCTATAAGGCGTCTAATTTAAAAAGTGGGGTCCCATCTAGATT |
| | | | CAGCGGCAGTGGATCTGGGACAGACTTCACTCTCACCATCAGCAGCCTGCAGCCTGATGAT |
| | | | GTTGCAACTTATTACTGTCAACAATATAATACTTACTCGGGGACGTTCGGCCAAGGGACCCG |
| | | | GGTGGAGATCAAAC |
| 7B6 | 61 | heavy | CAGGTGCAGCTACAGCAGTGGGGCGCAGGACTGTTGAAGCCTTCGGAGACCCTGTCCCTCA |
| | | | CCTGCGCTGTCTATGGTGGGTCCTTCAATGGTTACTACTGGAGTTGGATCCGCCAGCCCCCA |
| | | | GGGAAGGGGCTGGAGTGGATTGGGGAAATCGATCATAGTGGAAGCACCAACTACAACTCG |
| | | | TCCCTCAAGAGTCGAGTCACCATATCAGTAGACACGTCCAAGAACCAGTTCTCCCTGAAGCT |
| | | | GAGCTCCGTGACCGCCGCGGACACGGCTGTGTATTATTGCGCGAGGAGGCGCATTGGGAG |
| | | | CCTATTAAAATACTTCCAGGACTGGGGCCAGGGCACCCTGGTCACCGTCTCCTCAG |
| | 62 | light | TCCTATGAGCTGACTCAGCCACCCTCAGTGTCCGTGTCCCCAGGACAGACAGCCAGCATCAC |
| | | | CTGCTCTGGAGATAAATTGGGGGATAGTTATGTTTGTTGGTATCAACTGAAGCCAGGCCAG |
| | | | TCCCCTGTGCTGGTCATCTATCAAGATACCAAACGGCCCTCAGGGATCCCTGAGCGATTTTC |
| | | | TGGCTCCAACTCTGGGAACACAGCCACTCTGACCATCAGCGAGACCCAGGCTATGGATGAG |
| | | | GCTGACTATTACTGTCAGGCGTGGGACACCACCACGGATTGGGTGTTCGGCGGAGGGACC |
| | | | AAGCTGACCGTCCTAC |
| 8F3 | 63 | heavy | CAGCTGCAGCTGCAGGAGTCGGGCCCAGGCCTGGTGAAGCCTTCGGAGACCCTGTCCCTCA |
| | | | CCTGCACTGTGTCTGATGCCTCCATCGACACTCCGAGTTACTTCTGGAGCTGGATCCGCCAG |
| | | | CCCCCAGGGAAGGGGCTGGAGTGGATTGGCAGCATCTATTATACTGGGAACAAGTACTCCA |
| | | | ATCCGTCCCTCAAGAGTCGAGTCACCATGTCCGTAGACACGCCCAAGAGGCAGTTCTCCCTG |
| | | | AGGCTCAGCTCTGTGACCGCCGCAGACACGGCTGTTTATTACTGTGCGAGATATGTTGATTA |
| | | | TGTTTGGTTGAGGGCTTTTGATATATGGGGCCAAGGGACAAGGGTCACCGTCTCCTCAG |
| | 64 | light | GAAATTGTGTTGACACAGTCTCCAGCCACGCTGTCTTTGTCTCCAGGGGAAAGGGCCACCCT |
| | | | CTCATGCAGGGCCAGTCCGAGTGCTGGCCGCTTCTTAGCTTGGTACCAACAGAGACCTGGC |
| | | | CAGGCTCCCAGGCTCCTCATCTATGATGCATCCAAGAGGGCCACTGACACCCCAGCCAGGTT |
| | | | CAGTGGCAGCGGGTCTGGGACAGACTTCAATCTTACCATCGCCAGCCTAGAGCCTGAAGAT |
| | | | TTTGCAGTTTATTACTGTCAACACCGTAGCAACTGGCCGCTCACTTTCGGCGGAGGGACCAA |
| | | | GGTGGAGATCAAAC |
| 9A11 | 65 | heavy | GAGGTGCAGCTGGTGGAGTCCGGGGGAGGCTTAGTTCGGCCTGGGGGGTCCCTGAGACTC |
| | | | TCCTGTGTCGCCTCTGGATTCACCTTCAATACTTACTGGATGCACTGGGTCCGCCAAGCTCCA |
| | | | GGGAAGGGGCTACTATGGGTCTCATCTCTGAATAAAGATGGCAGTAGTCCAACGTACGCG |
| | | | GACTCCGTGAAGGGCCGGTTCACCATCTCCAGAGACAACACCAACAACACGCTCTTTCTCCA |
| | | | GTTGGACAGTCTGAGAGCCGAGGACACGGCTGTCTATTACTGTGTCAGAGATCGACGAGC |
| | | | GGTGACTACGGCCCGGTACTTCGATCTCTGGGGCCGTGGCACCCTCGTCACCGTCTCCTCAG |
| | 66 | light | GACATCGTGATGACCCAGTCTCCAGAGTCCCTGGCTGTGTCTCTGGGCGAGAGGGCCACCA |
| | | | TCAACTGCAAGTCCAGCTCCAACATTGATAACTACTTAGGTTGGTACCAGCAGAGACCAGG |
| | | | ACAGCCTCCTAAACTGCTCATTTACTGGGCATCTAAGCGGGAATCCGGGGTCCCTGACCGAT |
| | | | TCAGTGGCAGCGGGTCCGGGACAGATTTCACGCTCACCATCGGCCGCCTGCAGGCTGAAGA |
| | | | TGTGGCAGTTTATTACTGTCAACAATATTATACTAGTCCTCCGGTCACTTTTGGCGGAGGGA |
| | | | CCAAGGTGGAGATCAAAC |
| 15C2 | 67 | heavy | CAGGTGCAGCTGGTGCAATCTGAGTCTGAGTTGAAGAAGCCTGGGGCCTCAGTGAAGATTT |
| | | | CCTGCAAGGCTTCTGGATACGACCTCACTAAATATGCTATGAATTGGGTGCGGCAGGCCCC |
| | | | TGGACAAGGGCTTGAGTGGATGGGATGGATCAACACCAACACTGCGAAACCAACGTATGC |
| | | | CCAGGGCTTCACAGGACGATTTGTCTTCTCCTTGGACACGTCCGTCAGAACGATATATCTGG |
| | | | AGATCAGCAACCTAAAGGCTGAGGACACTGCCATTTATTACTGTTCGAGAGATGGTAGTGG |
| | | | GGCCTCCTGGGGCCAGGGAACCCTGGTCACCGTCTCCTCAG |
| | 68 | light | GACATCCAGATGACCCAGTCTCCTTCCACCCTGTCTGCATCTGTAGGAGACAGAGTCACCAT |
| | | | CACTTGCCGGGCCAGTCAGAATATTGGTCGCTGGTTGGCCTGGCATCAGCAGAAACCAGGG |
| | | | AAAGCCCCTAAGGTCCTGATCACTAAGGCGTCCAATTTAGAGAGTGGGGTCCCATCCAGGT |
| | | | TCACCGGCAGTGGCTCTGGGACAGACTTCACTCTCACCATCACCAGCCTGCAGCCTGATGAT |
| | | | TTTGCGACTTATTACTGTCAGCAATATACTACTTATTCGGGGACGTTCGGCCAAGGGACCAG |
| | | | GGTGGAAATCAAAC |
| 20M10 | 69 | heavy | GAGGTGCAGCTGGTGGAGTCTGGGGGAGGCCTGGTCAAGCCGGGGGGGTCCCTGCGACT |
| | | | CTCCTGTACAGTCTCGGGATTCACCTTCAATATGCATAGCATGAACTGGGTCCGCCAGGCTC |
| | | | CAGGGAAGGGGCTGGAGTGGGTCTCATTCATTAGTAGTACTAGCACTTACATCTACTATCCA |
| | | | GACTCAGTGAAGGGCCGATTCACCGTCTCCAGAGACAACTCCAAGAACTCACTGTATCTGC |
| | | | AAATGACCAGCCTGAGAGCCGAGGACACGGCTGTCTATTACTGTGTGAGACGAGGTCGGG |
| | | | GTGGAGCAGCCCGTGCACTTGACAACTGGGGCCAGGGTACCCTGGTCACCGTCTCCTCAG |
| | 70 | light | TCCTATGTTCTGCCTCAGACACCCTCGCTGTCAGTGGCCCCAGGACAGACGGCCACAATTTC |
| | | | CTGTGGGGGAAACAACACTGGAAGTAAAAGTGTGCACTGGTATCAACAGAAGCCAGGCCA |
| | | | GGCCCCTCTTCTGGTCGTCTACGATAATAGCGACCGGCCCTCAGGGATCCCTGAGCGATTCT |
| | | | CTGGGTCCAACTCTGGAAACACGGCCACCCTGACCCTCAGCAGGGTCGAAGCCGAGGATG |
| | | | AGGCCGACTATTACTGTCAGGTGTGGGATAGTACTATTGATCATGTGATATTCGGCGGCGG |
| | | | GACCAAGCTGACCGTCCTAG |
| 21C10 | 71 | heavy | GAGGTGCAGCTGGTGGAGTCTGGGGGAGGCCTGGTCAAGCCGGGGGGGTCCCTGAGACT |
| | | | CTCCTGTGCAGCCTCTGGATTCATTCTCAGTAGGAGTAGCATGAGCTGGGTCCGCCAGGCTC |
| | | | CAGGGAAGGGGCTGGAGTGGGTCTCATATATTAGTAGTACTAGTAGTCATATATATTATGC |
| | | | AGATTCATTGAAGGGCCGATTCACCATCTCCAGAGACAACACCGAGAACTCTGTATATCTGC |
| | | | AAATGAGTAGCCTGAGAGCCGAGGACACGGGTGTCTATTACTGTGCGAGAAAAACCAATG |
| | | | GGGCCTGGTTCCTCGATCTCTGGGGCCGTGGCACCCTGGTCACCGTCTCCTCAG |
| | 72 | light | CAGTCTGTGCTGACTCAGCCGCCCTCAGTGTCTGGGGCCCCAGGACAGAGGGTCACCATCT |
| | | | CCTGCACTGGGAGCAGCTCCAACATCGGGGCAGGTTCTGATGTGCACTGGTATCAGCAATT |
| | | | TCCAGGGTCAGCCCCCCACGTCCTCATCTATGGAAATAACCAACGGCCCTCAGGAGTCCCTG |
| | | | ACCGGTTCTCTGCCTCCAAGTCTGGCACCTCAGCCTCCCTGGCCATCACTGGGCTGCAGGCT |
| | | | GACGATGAGGCTGACTATTACTGCCAGTCCTATGACTACGACCTGAGTGGGTCTTGGGTCTT |
| | | | CGGCGGAGGGACCAAGCTGACCGTCCTGC |
| 22F2 | 73 | heavy | CAGGTGCAGCTGGTGCAATCTGAGTCTGAGTTGAAGAAGCCTGGGGCCTCAGTGAAGATTT |
| | | | CCTGCAAGGCTTCTGGATACGACCTCACTAAATATGCTATGAATTGGGTGCGGCAGGCCCC |
| | | | TGGACAAGGGCTTGAGTGGATGGGATGGATCAACACCAACACTGCGAAACCAACGTATGC |
| | | | CCAGGGCTTCACAGGACGATTTGTCTTCTCCTTGGACACGTCCGTCAGAACGATATATCTGG |
| | | | AGATCAGCAACCTAAAGGCTGAGGACACTGCCATTTATTACTGTTCGAGAGATGGTAGTGG |
| | | | GGCCTCCTGGGGCCAGGGAACCCTGGTCACCGTCTCCTCAG |
| | 74 | light | |
| 26A5 | 75 | heavy | CAGGTGCAGCTGGTGCAATCTGGGTCTGAGTTGAGGAAGCCTGGGGCCTCAGTGAAGGTT |
| | | | TCCTGCAAGGCTTCCGGATACACCTTCACTAAGTATGGTATGAATTGGGTGCGACAGGCCC |
| | | | CTGGACAAGGACTGGAGTGGATGGGATGGATTAACACGAACACTGCAAAGCCAACGTATG |
| | | | CCCAGGACTTCACAGGACGATTTGTCTTCTCTTTGGACACCTCTGTCAACACGGCATATCTG |
| | | | GAGATCAGCGGCCTAAAGGCTGAAGACACCGCCGTCTATTACTGTGCGACAGATGGTAGTG |
| | | | AGGGCTCCTGGGGCCAGGGAACCCTGGTCACCGTCTCCTCAG |
| | 76 | light | GACATCCAGATGACCCAGTCTCCTTCCACCCTGTCTGCATCTGTAGGAGACAGAGTCACCAT |
| | | | CACTTGCCGGGCCAGTCAGAGTATTGGTACCTGGTTGGCCTGGCATCAGCAGAAACCAGGG |
| | | | ACAGCCCCTAAGGTCCTGATCTATAAGGCGTCTAATTTAAAAAGTGGGGTCCCATCTAGATT |
| | | | CAGCGGCAGTGGATCTGGGACAGACTTCACTCTCACCATCAGCAGCCTGCAGCCTGATGAT |
| | | | GTTGCAACTTATTACTGTCAACAATATAATACTTACTCGGGGACGTTCGGCCAAGGGACCCG |
| | | | GGTGGAGATCAAAC |
| 3B7 | 77 | heavy | CAGGTGCAGCTGGTGCAATCTGGGTCTGAGTTGAAGAAGCCTGGGGCCTCAATGAAGGTT |
| | | | TCCTGCAAGGCTTCTGGATACACCTTCACTAACTATGCTATGAATTGGGTGCGACAGGCCCC |
| | | | TGGACAAGGGCTTGAGTGGATGGGATGGATCAACACCAACACTGGGAAACCAACGTATGC |
| | | | CCAGGGCTTCACAGGACGGTTTGTCTTCTCCTTGGACACCTCTGTCAGTATGGCATATCTGC |
| | | | AGATCAGCAGCCTAGAGGCTGGGGACACTGCCGTGTATTTCTGTGCGAGAGGCGAGTCCCT |
| | | | AGCAGCCCTCGGTTCGTTCGCCTACTGGGGCCAGGGAACCCTGGTCACCGTCTCCTCAG |
| | 78 | light | GACATCCAGATGACCCAGTCTCCATCCTCCCTGTCTGCATCTGTAGGAGACAGAGTCACCAT |
| | | | CACTTGCCGGGCAAGTCAGAGCATTAACAGCTATTTAGCTTGGTATCAGCAGAAACCAGGG |
| | | | AAAGCCCCTAACCTCCTGATCTATGCTGCATCCAATTTGCAAAGTGGGGTCCCATCACGGTT |
| | | | CAGTGGCAGTGGATCTGGGACAGATTTCACTCTCACCATCAGCAGTCTGCAACCTGAAGATT |
| | | | TTGCAACTTATTACTGTCAACAGAGTTCCAGTACTTTTTGGACGTTCGGCCAAGGGACCAAG |
| | | | GTGGAAATCAAAC |
| 7E6 | 79 | heavy | GAGGTGCAGTTGTTGGAGTCTGGGGGAGACTTGGTACATCCTGGGGGGTCCGTGAGACTC |
| | | | TCCTGTGCCGCCTCCTTTTTTAACTTTAGAATGTATCCCATGAGCTGGGTCCGCCAGGCTCCA |
| | | | GGGAAGGGGCTGGAGTGGGTCTCGACTATTAGTGGAACTGGTCAGACCACATACTATGCG |
| | | | GACTCCGTGCAGGGCCGCTTCACCATCTCCAGAGACAATTCCAACAACACTCTTTATCTACA |
| | | | CATGGGCAGCCTGCGAGCCGACGACACGGCCAAGTATTACTGTGCGCAACTGCATCTCGGT |
| | | | TATTATCATGAGAGCAGTGGTTATTTTTTAAGTTGGGGCCGGGGAACCCTGGTCACCGTCTC |
| | | | CTCAG |
| | 80 | light | GACATCCAGATGACCCAGTCTCCTTCCACCCTGTCTGCATCTGTAGGAGACACAGTCACCAT |
| | | | CACTTGCCGGGCCAGTCAGACTATAAGTAGTTGGTTGGCCTGGTATCAGCAGAAACCAGGA |
| | | | AAAGCCCCTAACCTCCTGCTTTATAAGGCGTCTACTTTAGAAAGTGGGGTCCCTTCAAGGTT |
| | | | CAGCGGCAGTGGATCTGGGACAGAATTCACTCTCACCATCAGAAGCCTGCAGCCTGAGGAT |
| | | | TTTGCAACTTATTACTGCCAACAGTATACTAGTTCTTGGACGTTCGGCCAAGGGACCAAGGT |
| | | | GGAAATCAAAC |
| 14C11 | 81 | heavy | CAGCTGCAGCTGCAAGAGTCGGGCCCAGGACTGGTGAAGCCTTCGGAGACCCTGTCCCTCA |
| | | | CCTGCACTGTCTCTGGTGGCTCCATCAGCAGTAGGACTTTCTTCTGGGGCTGGATCCGCCAG |
| | | | CCCCCAGGGAAGGGACTAGAGTGGATTGGGACTCTCTTTTCCGGTGGGACCACCTACCACA |
| | | | ACCCGTCCCTCACGAGTCGACTCACCATTTCCGTGGACACGTCCAGGAACCAGTTCTCCCTG |
| | | | AAACTGAGTTCTGTGACCGCCGCAGACACGGCTGTGTATTACTGTGCGAGACAACGGGCAG |
| | | | CATTACCGCCCTACTACTACTACTACTTCGACGTCTGGGGCAAAGGGACCACGGTCACCGTC |
| | | | TCCTCA |
| | 82 | light | GAAATTGTGTTGACACAGTCTCCAGCCACCCTGTCTTTGTCTCCAGGGGAAAGAGCCACCCT |
| | | | CTCCTGCAGGGCCAGTCAGAGTGTTGGCAGATACTTAGTCTGGTACCAACAGAAACCTGGC |
| | | | CAGGCTCCCAGGCTCCTCATCTATGATGCATCCAACAGGGCCACTGGCATCCCAGCCAGGTT |
| | | | CAGTGGCAGTGGGTCTGGGACAGACTTCACTCTCACCATCAGCAGCCTAGAGCCTGAAGAT |
| | | | TTTGCAGTTTATTACTGTCAGCAGCGTGGCAACTGGCCTCCGATCACCTTCGGCCAAGGGAC |
| | | | ACGACTGGAGATTAAAC |
| 31A12 | 83 | heavy | GAGGTGCAGCTGGTGGAGTCGGGGGGAGGCTTGGTGCGGCCTGGGGAATCCCTGAGACT |
| | | | CTCCTGTGAAGGCTCTGGATTCAGGTTCAATGAGCATAGTCTGAATTGGGTCCGCCAGGCT |
| | | | CCAGGGAAGGGGCTGGAGTGGCTTGCATACATTACTGACAGTAGCGGAAATACCATACACT |
| | | | ATGCAGACTCCGTGAAGGGCCGCTTCACCATCTCCAGAGACAACACCAAGAATTCAGTGTA |
| | | | TCTGCAGATGAACAGCCTGAGACCCGAAGACACGGCTGTGTATTACTGTGCGAGAGATGG |
| | | | GGGTTGTAATGGTCGCACCTGCTACGGACTCAACTACTGGGGCCAGGGAACCGTGGTCACC |
| | | | GTCTCCTCAG |
| | 84 | light | CAGTCTGTGTTGACGCAGACGCCCTCAATATCTGCGGCCCCAGGACAGAAGGTCACCATCT |
| | | | CCTGCTCTGGAAGCAGATCCGACATTGGGAATAGTTTTGTGTCCTGGTACCAGCAATTCCCA |
| | | | GGCTCAGCCCCCAAACTCCTCATTTATGACACTTTTAAGCGACCCTCTGGGATTCCTGACCGC |
| | | | TTCTCTGGCTCCAAGTCTGGCACGTCAGCCACACTGGCCATCACCGGGCTCCAGGCTGGGG |
| | | | ACGAGGCCGTTTATTACTGCGGAACATGGGATATCAGCCTGAGTGCGGCGGTCTTCGGCGG |
| | | | AGGGACCATGCTGACCGTCCTAG |
| 20G11 | 85 | heavy | CAGGTGCAGCTGGTGGAGTCTGGGGGAGGCGTGGTCCAGCCTGGGGGGTCCCTGAGACTC |
| | | | TCCTGTGCAACGTCTGGATTCACCTTCAGTAGTTATGGCATGCACTGGGTCCGCCAGGCTCC |
| | | | AGGCAAGGGGCTGGAGTGGGTGGCATTTATACGGTATAATGGAAGTAATAAATACTATGC |
| | | | AGACTCCGTGAAGGGCCGATTCACCATCTCCAGAGACACTTCCAAGAACACGCTGTATCTGC |
| | | | AAATGAACAGCCTGAGAGCTGAGGACTCGGCTGTGTATTACTGTGCGAAAGACGGCGATT |
| | | | ACGATTCTTGGAGTGGTCTCACTGAACACTTCCAGCACTGGGGCCAGGGCACCCTGGTCAC |
| | | | CGTCTCCTCAG |
| | 86 | light | GACATCGTGATGACCCAGTCTCCAGACTCCCTGGCTGTGTCTCTGGGCGAGAGGGCCACCA |
| | | | TCAACTGCAAGTCCAGCCAGAATGTTTTATACAGCTCCAACAATAAGAACTACTTAGCTTGG |
| | | | TACCAGCAGAAACCAAGACAGCCTCCTAAGCTGCTCATTTACTGGGCATCTACCCGGGAATC |
| | | | CGGGGTCCCTGACCGATTCAGTGGCAGCGGGTCTGGGACAGATTTCACTCTCACCATCAGC |
| | | | AGCCTGCAGGCTGAAGATGTGGCAGTTTATTACTGTCAGCAATATTATAGTATACCTCCGGC |
| | | | TTTCGGCCCTGGGACCAAAGTGGAAATCAAAC |
| 9H11 | 87 | heavy | CAGGTGCAGCTGATGGAGTCTGGGGGAGGCGTGGTCCAGCCGGGGGGGTCCCTGAGACT |
| | | | CTCCTGTGCGGCGTCTGGATTCGACTTCCCTGGCTATGGCATGCACTGGGTCCGCCAGACTC |
| | | | CCGACAAGGGGCTGGAGTGGGTGGCATATATATGGTACGATGCCAGAAGTGAAGACTATG |
| | | | TAGACTCCGTGAAGGGCCGATTCACCATTTCCAGAGACAACTCCAACAACACGTTGTATCTC |
| | | | AAGATGACCGATCTGAGACCTCAGGACACGGCTATGTATTATTGTGCGGTGGGAGCTCCTC |
| | | | TCGAAGGGTACTGGGGCCAGGGAACCCGGGTCACCGTCTCCTCAG |
| | 88 | light | GACATCCAGATGACCCAGTCTCCATCCTCCCTGTCTGCATCTGTAGGGGACAAGGTCACCAT |
| | | | CACTTGCCGGGCAAGTCAGAACATTGCCGACTATTTAAGTTGGTTTCAGCAAAAACCAGGG |
| | | | AAAGCCCCTAAAATCCTGATCTATGCTGCATCTACTTTGCAAAGTGGGGTCCCATCAAGGTT |
| | | | CAGTGGCAGTGGATCTGGGACACATTTCACTCTCACCATCAGCAGTCTGCAGCCCGAAGATT |
| | | | TTGCAAGTTACTTCTGTCAGCAGAGTTACACTTCACCCACGTGGACGTTCGGCCAAGGGACC |
| | | | AAGGTGGAAGTCAAAC |
| 26C3 | 89 | heavy | CAGGTGCAGCTGCAGGAGTCGGGCCCAGGACTGGTGAAGCCTTOGGAGACCCTGTOCCTCA |
| | | | CCTGCACTGTCTOTGGTGGCTOCATCAGTAGTTACTACTGGACCTGGATCCGGCAGCCCGCC |
| | | | GGGAAGGGACTGGAGTGGATTGGGCGTATCTTTACCACTGGGAGCACCAATTATAATCCCTC |
| | | | CCTCAAGAGTCGAGTCACCATGTCAGTAGACACGTCCAAGAACCAGTTCTCCCTACACCTGAC |
| | | | CTCTGTGACCGCCGCGGACACGGCCGTATATTATTGTGCGAGACTGAGGAGGGTAGTACCAA |
| | | | CTGCTATCTGGCACTTOGATOTCTGGGGCCGTGGCACCOGGGTCACCGTCTCCTCAG |
| | 90 | light | CAGACTGTGGTGACCCAGGAGCCATCGATCTCAGTGTCCCCTGGAGGGACAGTCACACTCAC |
| | | | TTGTGGCTTGAGCTCTGGCGCAGTCTCTACTAGTTACTTCCCCAGCTGGTACCAGCAGATCCC |
| | | | AGGCCAGGCACCACGCACGCTCATCTACGGCACAAACACTCGCTCTTCTGGGGTCCCTGATC |
| | | | GCTTCTCTGGCTCCATCCTTGGGAACAAAGCTGCCCTCACCATCACGGGGGCCCAGGCAGAT |
| | | | GATGAATGTGATTATTACTGTGTGTTGTATATGGATAGTGGCGTGATGGTATTCGGCGGAGGG |
| | | | ACCAAGCTGACCGTCCTAG |

TABLE 2
PROTEIN SEQUENCES FOR ANTIBODY VARIABLE REGIONS

| | SEQ ID | | |
|---|---|---|---|
| Clone | NO: | Chain | Variable Sequence |
| 40C7 | 129 | heavy | EVQLVESGGDLVQPGGSLRLSCAASRFSFGSYAMSWVRQ |
| | | | APGKGLEWVSAISSSGGRAYYADSVKGRFTISRDNSRNTL |
| | | | YLQLNSLRGEDTAFYYCTKEGDTGSLWLFDSWGQGTLVT |
| | | | VSS |
| | 130 | light | DIVMTQSPDSLAVSLGERATINCKSSQSVLDSSNNKKYLA |
| | | | WYQQKPGQPPKLLIYWASTREFGVPDRFSGSGSGTDFTL |
| | | | TISSLQAEDVAVYYCQQYYSTPPTFGQGTKVEIK |
| 3B10 | 131 | heavy | EVQLVESGGALVKPGGSLRLSCEGSGFMFSSAWLHWVRQ |
| | | | APGMGLELVGRMKSETDGGTLDYTAPVRGRFTISRDDSKN |
| | | | MFFLQMNSLKAEDTAVYYCVTGNFDYRSSPLGFWGQGAL |
| | | | VTVSS |
| | 132 | light | DIQMTQSPSTLSASAGDRVTITCRASENIYKWLAWYQQKP |
| | | | GKAPKLLIYKVSTLESGVPSRFSGSGFGTEFTLTISSLQPGD |
| | | | FATYYCQHYNSFPFTFGQGTKLEVK |
| 4G4 | 133 | heavy | EVQLVQSGGGLVQPGGSLRLSCAASRFIFGSYAMSWVRQ |
| | | | APGKGLEWVSAISSSGGRTYYADSVKGRFTISRDNSRNTL |
| | | | YLQLSSLRAEDTAFYYCTKEGDTGSLWLFDSWGQGTLVTV |
| | | | SS |
| | 134 | light | DIVMTQSPDSLAVSLGERATINCKSSQSVLDSSNNKKYLA |
| | | | WYQQKPGQSPKLLIYWASTREFGVPDRFSGSGSGTDFTL |
| | | | TISSLQAEDVAVYYCQQYYTTPPTFGRGTKVEIK |
| 16G12 | 135 | heavy | EVQLVESGGGLVKPGGSLKLSCAASGFTFSDYYMYWVRQ |
| | | | TPEKRLEWVATISDGGSYTYYPDNVKGRFTISRDNAKNTLY |
| | | | LQMSHLKSEDTAMYYCARDKGPIVPMPMDYWGQGTSVTV |
| | | | SS |
| | 136 | light | SYELTQPLSVSVSPGQTVNITCSGDKLGAKFTCWYQQKPG |
| | | | QSPILVLYQDTKRPPGIPERFSGSNSGNTATLTISGTQAMD |
| | | | EADYYCQAWESGIVAFGGGTKLTVV |
| 15B8 | 137 | heavy | QVQLVESGGRVVQPGESLRLSCETSGFPFHDHGMHWVR |
| | | | QAPGKGLEWVAFIRFDSTSKYTSDSVRGRFSISRDNSKSTL |
| | | | FLQMNNLRREDTAIYYCARDANARFGVMIMAHWGQGTLV |
| | | | TVSS |
| | 138 | light | DIQLTQSPSSLSASVGDRVTITCQASQDIGHYLNWYLQKPG |
| | | | QAPQVLIYDASNLVTGVPSRFSGSGSGTHFTFTISSLQPED |
| | | | FGTYYCHQHEKLYSISFGQGTRLDIK |
| 15A7 | 139 | heavy | QVQLVQSGPEVRKPGASVKVSCKTSGYMFTDYEMNWVR |
| | | | QAPGQGLEWMGLINPHSGDTGYAQKFQGRVSMTSDTSTR |
| | | | TFYLELTGLTSEDTAVYYCARSQGTFEVYYFVSWGQGSLV |
| | | | TVSS |
| | 140 | light | SYVLTQPPSVSVAPGKTASITCGAKNIATKRVHWYRQKPG |
| | | | QAPVVVVSDEEDRSSVIPERFSGSKSGDTATLEISRVEAGD |
| | | | EADYYCQVWDSLADQVVFGGGTKVTVL |
| 5D7 | 141 | heavy | QVQLVQSGSELKKPGASVKVSCEASGYNLTTYAMNWVRQ |
| | | | APGQGLEWMGWINTNTGNPTYAQDFTGHFVFSLDTSVST |
| | | | AYLQISSLKAEDTAVYYCARDFGERGNCVNGVCYGGYGM |
| | | | DVWGQGTTVTVSS |
| | 142 | light | DIQMTQSPSTLSASVGDRVTITCRASQSISRWLAWYQQKP |
| | | | GKVPKLLIYKASRLEGGVPSRFSGSGSGTEFTLTISSLQPD |
| | | | DFATYYCQEYTNYSGTFGQGTKVEIR |
| 14B3 | 143 | heavy | QVQLVQSGPEVKKPGASVKVSCKTSGYTFTDSEMNWVRQ |
| | | | APGQGLEWMGLINPHSGDTRYAERFQGRVTMTSDTSINTV |
| | | | YLELSGLTSEDTAIYFCARSQGTFEVYYFLSWGQGSLITVS |
| | | | S |
| | 144 | light | |
| 2C9 | 145 | heavy | QVQLVESGGGLVKPGGSLRLSCAASGFSFSDFYMSWIRQ |
| | | | APGKGLECISYMSSSGGNIYYADSVKGRFTISRDNAKNSLS |
| | | | LQMNSLRADDTALYYCARRRYGSGSSIFDYWSQGTLVTVS |
| | | | S |
| | 146 | light | SYELTQPPSVSVSPGQTASVTCSGDTLGDRYVSWYQQKA |
| | | | GQSPVLVIYQSGQRPSGIPERFSGSNSGNTATLSISETQAL |
| | | | DEADYYCLTWDRGTPVFGPGTTVTVV |
| 2F12 | 147 | heavy | QVQLVQSGPEVKKPGASVKVSCQTSGYTFTDYEMNWVR |
| | | | QAPGQGLEWMGLINPHSGDTGYALKFQGRVTMTSDTSKS |
| | | | TVYLELSGLTSEDTAVYYCARSQGTFEVYYFVSWGQGSLV |
| | | | TVSS |
| | 148 | light | SYVLTQPPSVSVAPGKTATITCGANGIGRKRVHWYSQRPG |
| | | | QAPVVVVSDDDDRSSVNPERFSASKSGDTAALTITRVEAG |
| | | | DEADYYCQVWDSRTDQVVFGGGTKVTVL |
| 29B3 | 149 | heavy | QVQLVQSGPEVKKPGASVKVSCKTSGYSFTDYEMNWVRQ |
| | | | APGQGLEWIGLVNPHSGETGYALKFQGRVTMTSDTAISTV |
| | | | YLELSGLTTEDTAIYYCARSQGTFEVYYFVSWGQGSLVTV |
| | | | SS |
| | 150 | light | |
| 38B7 | 151 | heavy | QVQLVESGGGVVQPGRSLRLSCVGSGFTFRRDAKYWVR |
| | | | QAPGKGLEWVAAISHDGGEEDYAESVKGRFTISRDNSRET |
| | | | VYLEMNSLRPEDTAVYYCATTRSGWYFDYWGQGALVTVS |
| | | | S |
| | 152 | light | EIVLTQSPATLSLSPGARATLSCRASQSVDTYLAWYQQRR |
| | | | GQSPRLLIYDASKRATGIPARFSGSGSGTDFTLTISSLEPED |
| | | | FAVYYCQQRNNWRTWTFGQGTKVEIN |
| 16A8 | 153 | heavy | QVQLVQSGPDVRKPGATVKVSCQTSGYRFTDYEMNWVR |
| | | | QAPGQGLEWIGLINPHSGDTAYAQKFQGRVTMTSDTSDST |
| | | | VYLELSGLTPDDTAVYYCARSQGTFEVYYFVSWGQGSLVT |
| | | | VSS |
| | 154 | light | SYVLTQPPSMSVAPGTTASIPCGANNIAKKRVHWYRQKPG |
| | | | QAPVVVVSDDEDRSSVIPDRFSGSKSGDTATLTISRVEAGD |
| | | | EGDYYCQVWDSKTDHVVFGGGTKVTVL |
| 11F10 | 155 | heavy | EVQLVESGGGLVQPGRSLRLSCAASGFTFDDYTMHWVRQ |
| | | | APGKGLEWVSSIRWNSGNLDYADSVKGRFTISRDNARHSL |
| | | | YLQMNSLRAEDTALYYCVKDNGLRTLDFWGQGTLVTVSS |
| | 156 | light | DIVMTQSPLSLPVTPGEPASISCRSSQSLLHRNGYNYLDW |
| | | | YLQKPGQSPQLLISLASNRASGVPDRFSGSGSGTDFTLKIS |
| | | | RVEAEDVGVYYCMQALQTWTFGQGTKVELK |
| 32G9 | 157 | heavy | QVQLVQSGPEVRKPGASVRVSCTTSGYTFIDYEMNWVRQ |
| | | | APGQGLEWMGLINPHSGDTGYAQKFQDRVTMTSDTSLST |
| | | | AYLELSGLTSEDTAVYYCARSQGTFEVYYFVSWGQGSLVT |
| | | | VSS |
| | 158 | light | SYVLTQPPSVSVAPGKTASITCEAKNIVRKRVHWYRQKPG |
| | | | QAPVVVVSDDEDRSSVIPERVSGSKSGDTATLTISRVEAGD |
| | | | EADYYCQVWDSTTDQVVFGGGTKVTVL |
| 24F10 | 159 | heavy | QVQLVQSGPEVKKPGASVKVSCQTSGYTFTDYEMNWVR |
| | | | QAPGQGLEWMGLINPHSGDTGYALKFQGRVTMTSDTSKS |
| | | | TVYLELSGLTSEDTAVYYCARSQGTFEVYYFVSWGQGSLV |
| | | | TVSS |
| | 160 | light | |
| 15A4 | 161 | heavy | |
| | 162 | light | DIVLTQSPDSLAVSLGERATINCKSSQNILDNSNNKNFIAWH |
| | | | QHKPGQPPKLLIYWGSSRESGVPDRFSGSGSGTDFTLTIS |
| | | | NLQAEDVAVYYCLQYYSLPHTFGQGTKVEIK |
| 13D9 | 163 | heavy | QVQLVESGGGLVKPGGSLRLSCVASGFTFSDFYMSWIRQ |
| | | | APGKGLEWVSYMSATGGNIYYADSMKGRLTISRDNTKNSL |
| | | | FLQMNSLRADDTALYYCARRKFGAGSAIFDHWSQGTLVTV |
| | | | SS |
| | 164 | light | SYELTQPPSVSVSPGQTASVTCSGDKLGERYVSWYQQKA |
| | | | GQSPDLVIYQTNQRPSGIPERFSGSDSGNTATLTISGTQGL |
| | | | DEADYYCLTWDRGTPVFGTGTKVTVL |
| 5C5 | 165 | heavy | EVQLLESGGGLVQPGGSLRLSCAASGFTFSNHAMSWVRQ |
| | | | TPGEGLQWVSALTYSGKTTYYADSVKGRFTISRDNSKNLL |
| | | | FLQMNSLRAGDTAIYYCAKEDYDDRGFFDFWGQGTRVTV |
| | | | SS |
| | 166 | light | DIQMTQSPSSLSASVGDRVTITCRASQTISTYLHWYQQKP |
| | | | GKAPNLLIYAASTLQSGVPSRFSGSGSGTDFSLTISSLRPE |
| | | | DFAIYYCQQGYNNPYTFGQGTKVDIK |
| 14G12 | 167 | heavy | QVQLVESGGGVVQPGRSLRLSCAGSGFRFSDYGMHWVR |
| | | | QAPGKGLEWVAIVAYDESKKYYADSVKGRFTISRDNSKNT |
| | | | LFLQMNSLRADDTAVYYCAKRTQKFAPYYFNGLDVWGQG |
| | | | TTVTVSS |
| | 168 | light | DIVMTQSPLSLPVTPGEPASISCRSSQSLLDRNGYNYLDW |
| | | | YVQKPGQSPQLLMYLGSDRASGVPARFSGSGSGTDFTLKI |
| | | | SRVEVEDVGVYYCMQALQIPITFGQGTRLEIK |
| 17D7 | 169 | heavy | QVQLVESGGGVVQPGRSLRLSCAGSGFRFSDYGMHWVR |
| | | | QAPGKGLEWVAIVAYDESKKYYADSVKGRFTISRDNSKNT |
| | | | LFLQMNSLRADDTAVYYCAKRTQKFAPYYFNGLDVWGQG |
| | | | TTVTVSS |
| | 170 | light | DIVMTQSPLSLPVTPGEPASISCRSSQSLLDRNGYNYLDW |
| | | | YVQKPGQSPQLLMYLGSDRASGVPARFSGSGSGTDFTLKI |
| | | | SRVEVEDVGVYYCMQALQIPITFGQGTRLEIK |
| 3C3 | 171 | heavy | QLQLQESGPGLVKPSETLSLTCSISGASINSDDYYWGWIR |
| | | | QAPGEALEWIASIDASGTTFYNPSLRSRVTISVDTSTNQISL |
| | | | RLNSVTAADTAIYYCARRKTYYDFLTDYYNWYFDVWGR |
| | | | GTLVTVSS |
| | 172 | light | QSALTQPRSVSGSPGQSVTFSCTGINSDIRDYDYVSWYQQ |
| | | | HPGEAPKLIIYDVTKRASGVPSRFSGSKSGNTASLTISGLQ |
| | | | AEDEADYYCCSYSVTYSFEVFGTGTQVSVL |
| 17G11 | 173 | heavy | QVQLVESGGGVVQPGRSLRLSCAGSGFRFSDYGMHWVR |
| | | | QAPGKGLEWVAIVAYDESKKYYADSVKGRFTISRDNSKNT |
| | | | LFLQMNSLRADDTAVYYCAKRTQKFAPYYFNGLDVWGQG |
| | | | TTVTVSS |
| | 174 | light | DIVMTQSPLSLPVTPGEPASISCRSSQSLLDRNGYNYLDW |
| | | | YVQKPGQSPQLLMYLGSDRASGVPARFSGSGSGTDFTLKI |
| | | | SRVEVEDVGVYYCMQALQIPITFGQGTRLEIK |
| 15C4 | 175 | heavy | EVQLVESGGGLVKPGGSLRLSCAASGFTFSLYTMNWVRQ |
| | | | APGKGLEWVSSITSNSSHLYYVDSVKGRFTISRDNSKNSV |
| | | | SLQMNSLRAEDTAVYYCTRSYSSKYDNWFDPWGQGMLV |
| | | | TVSS |
| | 176 | light | SYELTQPPSVSVSPGQTASIMCSGDNLGDKYVCWYQQKP |
| | | | GHSPELVMYRDNKRPSGVPERFSGSKSGNTATLTISGSQA |
| | | | MDEADYYCQALDSGSFWVFGGGTKLTVL |
| 24C8 | 177 | heavy | QVQLVESGGGVVQPGRSLRLSCAGSGFRFSDYGMHWVR |
| | | | QAPGKGLEWVAIVAYDESKKYYADSVKGRFTISRDNSKNT |
| | | | LFLQMNSLRADDTAVYYCAKRTQKFAPYYFNGLDVWGYG |
| | | | TTVTVSS |
| | 178 | light | DIVMTQSPLSLPVTPGEPASISCRSSQSLLDRNGYNYLDW |
| | | | YVQKPGQSPQLLMYLGSDRASGVPARFSGSGSGTDFTLKI |
| | | | SRVEVEDVGVYYCMQALQIPITFGQGTRLEIK |
| 1A8 | 179 | heavy | QLQLQESGPGLVKPSETLSLTCSVSDDSISTPSYFWTWIRQ |
| | | | PPGKGLEWIASIYYTGTTYYNPSLKSRVTLSVDTPKRQFFL |
| | | | RLSSVTAADTAVYYCARYLDYVWLRAFDVWGQGAMVTVS |
| | | | S |
| | 180 | light | EIVLTQSPATLSLSPGERATLSCRASPSVGRFLAWYQQKP |
| | | | GQAPRLLIYDASQRATDIPARFSASGSGTDFTLTIDSLEPED |
| | | | FAIYYCQHRSNWPVTFGGGTRVEIK |
| 3F9 | 181 | heavy | EVQLVESGGGLVKPGGSLRLSCIGSGFDFSRDTFHWVRQ |
| | | | APGKGLEWISSISRIETYTYYVDSVKGRFTVSRDNAKNSVY |
| | | | LQMNSLRVEDTAVYFCARAGAVRPGIGFHYFDNWGQGSP |
| | | | VTVSS |
| | 182 | light | |
| 6E10 | 183 | heavy | |
| | 184 | light | DIQMTQSPSTVSASVGDRVTITCRASQSIGRWLAWHQQKP |
| | | | GKAPKVLITRASNVESGVPSRFSGSGSGTEFTLTISSLQPD |
| | | | DFATYYCQQYNTNSGTSGQGTKAEIK |
| 4D11 | 185 | heavy | QVQLVESGGGLVKPGGSLRLSCTASGFNFTNYYMSWIRQ |
| | | | APGKGLEWVSYISSTTNSIDYADSVKGRFTISRDNAKKSLY |
| | | | LQMNSLRADDTAFYYCARHLVRGTSLAAFDIWGQGTMVTV |
| | | | SS |
| | 186 | light | |
| 1H9 | 187 | heavy | QVQLVQSGSELRKPGASVKVSCKASGYTFTKYGMNWVRQ |
| | | | APGQGLEWMGWINTNTAKPTYAQDFTGRFVFSLDTSVNT |
| | | | AYLEISGLKAEDTAVYYCATDGSEGSWGQGTLVTVSS |
| | 188 | light | DIQMTQSPSTLSASVGDRVTITCRASQSIGTWLAWHQQKP |
| | | | GTAPKVLIYKASNLKSGVPSRFSGSGSGTDFTLTISSLQPD |
| | | | DVATYYCQQYNTYSGTFGQGTRVEIK |
| 7B6 | 189 | heavy | QVQLQQWGAGLLKPSETLSLTCAVYGGSFNGYYWSWIRQ |
| | | | PPGKGLEWIGEIDHSGSTNYNSSLKSRVTISVDTSKNQFSL |
| | | | KLSSVTAADTAVYYCARRRIGSLLKYFQDWGQGTLVTVSS |
| | 190 | light | SYELTQPPSVSVSPGQTASITCSGDKLGDSYVCWYQLKPG |
| | | | QSPVLVIYQDTKRPSGIPERFSGSNSGNTATLTISETQAMD |
| | | | EADYYCQAWDTTTDWVFGGGTKLTVL |
| 8F3 | 191 | heavy | QLQLQESGPGLVKPSETLSLTCTVSDASIDTPSYFWSWIRQ |
| | | | PPGKGLEWIGSIYYTGNKYSNPSLKSRVTMSVDTPKRQFS |
| | | | LRLSSVTAADTAVYYCARYVDYVWLRAFDIWGQGTRVTV |
| | | | SS |
| | 192 | light | EIVLTQSPATLSLSPGERATLSCRASPSAGRFLAWYQQRP |
| | | | GQAPRLLIYDASKRATDTPARFSGSGSGTDFNLTIASLEPE |
| | | | DFAVYYCQHRSNWPLTFGGGTKVEIK |
| 9A11 | 193 | heavy | EVQLVESGGGLVRPGGSLRLSCVASGFTFNTYWMHWVR |
| | | | QAPGKGLLWVSSLNKDGSSPTYADSVKGRFTISRDNTNNT |
| | | | LFLQLDSLRAEDTAVYYCVRDRRAVTTARYFDLWGRGTLV |
| | | | TVSS |
| | 194 | light | DIVMTQSPESLAVSLGERATINCKSSSNIDNYLGWYQQRP |
| | | | GQPPKLLIYWASKRESGVPDRFSGSGSGTDFTLTIGRLQA |
| | | | EDVAVYYCQQYYTSPPVTFGGGTKVEIK |
| 15C2 | 195 | heavy | QVQLVQSESELKKPGASVKISCKASGYDLTKYAMNWVRQ |
| | | | APGQGLEWMGWINTNTAKPTYAQGFTGRFVFSLDTSVRTI |
| | | | YLEISNLKAEDTAIYYCSRDGSGASWGQGTLVTVSS |
| | 196 | light | DIQMTQSPSTLSASVGDRVTITCRASQNIGRWLAWHQQKP |
| | | | GKAPKVLITKASNLESGVPSRFTGSGSGTDFTLTITSLQPD |
| | | | DFATYYCQQYTTYSGTFGQGTRVEIK |
| 20M10 | 197 | heavy | EVQLVESGGGLVKPGGSLRLSCTVSGFTFNMHSMNWVRQ |
| | | | APGKGLEWVSFISSTSTYIYYPDSVKGRFTVSRDNSKNSLY |
| | | | LQMTSLRAEDTAVYYCVRRGRGGAARALDNWGQGTLVTV |
| | | | SS |
| | 198 | light | SYVLPQTPSLSVAPGQTATISCGGNNTGSKSVHWYQQKP |
| | | | GQAPLLVVYDNSDRPSGIPERFSGSNSGNTATLTLSRVEA |
| | | | EDEADYYCQVWDSTIDHVIFGGGTKLTVL |
| 21C10 | 199 | heavy | EVQLVESGGGLVKPGGSLRLSCAASGFILSRSSMSWVRQ |
| | | | APGKGLEWVSYISSTSSHIYYADSLKGRFTISRDNTENSVY |
| | | | LQMSSLRAEDTGVYYCARKTNGAWFLDLWGRGTLVTVSS |
| | 200 | light | QSVLTQPPSVSGAPGQRVTISCTGSSSNIGAGSDVHWYQ |
| | | | QFPGSAPHVLIYGNNQRPSGVPDRFSASKSGTSASLAITGL |
| | | | QADDEADYYCQSYDYDLSGSWVFGGGTKLTVL |
| 22F2 | 201 | heavy | QVQLVQSESELKKPGASVKISCKASGYDLTKYAMNWVRQ |
| | | | APGQGLEWMGWINTNTAKPTYAQGFTGRFVFSLDTSVRTI |
| | | | YLEISNLKAEDTAIYYCSRDGSGASWGQGTLVTVSS |
| | 202 | light | |
| 26A5 | 203 | heavy | QVQLVQSGSELRKPGASVKVSCKASGYTFTKYGMNWVRQ |
| | | | APGQGLEWMGWINTNTAKPTYAQDFTGRFVFSLDTSVNT |
| | | | AYLEISGLKAEDTAVYYCATDGSEGSWGQGTLVTVSS |
| | 204 | light | DIQMTQSPSTLSASVGDRVTITCRASQSIGTWLAWHQQKP |
| | | | GTAPKVLIYKASNLKSGVPSRFSGSGSGTDFTLTISSLQPD |
| | | | DVATYYCQQYNTYSGTFGQGTRVEIK |
| 3B7 | 205 | heavy | QVQLVQSGSELKKPGASMKVSCKASGYTFTNYAMNWVR |
| | | | QAPGQGLEWMGWINTNTGKPTYAQGFTGRFVFSLDTSVS |
| | | | MAYLQISSLEAGDTAVYFCARGESLAALGSFAYWGQGTLV |
| | | | TVSS |
| | 206 | light | DIQMTQSPSSLSASVGDRVTITCRASQSINSYLAWYQQKP |
| | | | GKAPNLLIYAASNLQSGVPSRFSGSGSGTDFTLTISSLQPE |
| | | | DFATYYCQQSSSTFWTFGQGTKVEIK |
| 7E6 | 207 | heavy | EVQLLESGGDLVHPGGSVRLSCAASFFNFRMYPMSWVRQ |
| | | | APGKGLEWVSTISGTGQTTYYADSVQGRFTISRDNSNNTL |
| | | | YLHMGSLRADDTAKYYCAQLHLGYYHESSGYFLSWGRGT |
| | | | LVTVSS |
| | 208 | light | DIQMTQSPSTLSASVGDTVTITCRASQTISSWLAWYQQKP |
| | | | GKAPNLLLYKASTLESGVPSRFSGSGSGTEFTLTIRSLQPE |
| | | | DFATYYCQQYTSSWTFGQGTKVEIK |
| 14C11 | 209 | heavy | QLQLQESGPGLVKPSETLSLTCTVSGGSISSRTFFWGWIR |
| | | | QPPGKGLEWIGTLFSGGTTYHNPSLTSRLTISVDTSRNQFS |
| | | | LKLSSVTAADTAVYYCARQRAALPPYYYYYFDVWGKGTTV |
| | | | TVSS |
| | 210 | light | EIVLTQSPATLSLSPGERATLSCRASQSVGRYLVWYQQKP |
| | | | GQAPRLLIYDASNRATGIPARFSGSGSGTDFTLTISSLEPED |
| | | | FAVYYCQQRGNWPPITFGQGTRLEIK |
| 31A12 | 211 | heavy | EVQLVESGGGLVRPGESLRLSCEGSGFRFNEHSLNWVRQ |
| | | | APGKGLEWLAYITSSGNTIHYADSVKGRFTISRDNTKNSVY |
| | | | LQMNSLRPEDTAVYYCARDGGCNGRTCYGLNYWGQGTV |
| | | | VTVSS |
| | 212 | light | QSVLTQTPSISAAPGQKVTISCSGSRSDIGNSFVSWYQQFP |
| | | | GSAPKLLIYDTFKRPSGIPDRFSGSKSGTSATLAITGLQAGD |
| | | | EAVYYCGTWDISLSAAVFGGGTMLTVL |
| 20G11 | 213 | heavy | QVQLVESGGGVVQPGGSLRLSCATSGFTFSSYGMHWVR |
| | | | QAPGKGLEWVAFIRYNGSNKYYADSVKGRFTISRDTSKNT |
| | | | LYLQMNSLRAEDSAVYYCAKDGDYDSWSGLTEHFQHWG |
| | | | QGTLVTVSS |
| | 214 | light | DIVMTQSPDSLAVSLGERATINCKSSQNVLYSSNNKNYLA |
| | | | WYQQKPRQPPKLLIYWASTRESGVPDRFSGSGSGTDFTL |
| | | | TISSLQAEDVAVYYCQQYYSIPPAFGPGTKVEIK |
| 9H11 | 215 | heavy | QVQLMESGGGVVQPGGSLRLSCAASGFDFPGYGMHWVR |
| | | | QTPDKGLEWVAYIWYDARSEDYVDSVKGRFTISRDNSNNT |
| | | | LYLKMTDLRPQDTAMYYCAVGAPLEGYWGQGTRVTVSS |
| | 216 | light | DIQMTQSPSSLSASVGDKVTITCRASQNIADYLSWFQQKP |
| | | | GKAPKILIYAASTLQSGVPSRFSGSGSGTHFTLTISSLQPED |
| | | | FASYFCQQSYTSPTWTFGQGTKVEVK |
| 26C3 | 217 | heavy | QVQLQESGPGLVKPSETLSLTCTVSGGSISSYYWTWIRQP |
| | | | AGKGLEWIGRIFTTGSTNYNPSLKSRVTMSVDTSKNQFSL |
| | | | HLTSVTAADTAVYYCARLRRVVPTAIWHFDLWGRGTRVTV |
| | | | SS |
| | 218 | light | QTVVTQEPSISVSPGGTVTLTCGLSSGAVSTSYFPSWYQQI |
| | | | PGQAPRTLIYGTNTRSSGVPDRFSGSILGNKAALTITGAQA |
| | | | DDECDYYCVLYMDSGVMVFGGGTKLTVL |

TABLE 3
CDR HEAVY CHAIN SEQUENCES

| | | (SEQ ID | | (SEQ ID | | (SEQ ID |
|---|---|---|---|---|---|---|
| Clone | CDRH1 | NO:) | CDRH2 | NO: ) | CDRH3 | NO: ) |
| 40C7 | agattcagctttg | 248 | atttcttctagtg | 249 | acgaaagaaggtgaca | 250 |
| | gcagctatgcc | | gtggtagggca | | cagggtccctatggttattt | |
| | | | | | gactcc | |
| | RFSFGSYA | 251 | ISSSGGRA | 252 | TKEGDTGSLWLF | 253 |
| | | | | | DS | |
| 3B10 | ggtttcatgttcag | 254 | atgaaaagtga | 255 | gtcacaggaaacttcgat | 256 |
| | tagcgcctgg | | gactgatggtg | | tataggagttcaccccttg | |
| | | | ggacactt | | gcttc | |
| | GFMFSSAW | 257 | MKSETDG | 258 | VTGNFDYRSSPLG | 259 |
| | | | GTL | | F | |
| 4G4 | agattcatctttgg | 260 | attagttctagtg | 261 | acgaaagaaggtgaca | 262 |
| | cagctatgcc | | gtggcaggac | | cagggtccctatggcttttt | |
| | | | a | | gactcc | |
| | RFIFGSYA | 263 | ISSSGGRT | 264 | TKEGDTGSLWLF | 265 |
| | | | | | DS | |
| 16G12 | ggattcactttca | 266 | attagtgatggt | 267 | gcaagagataaagggc | 268 |
| | gtgactattac | | ggtagttacac | | ctatagtccctatgcctat | |
| | | | C | | ggactac | |
| | GFTFSDYY | 269 | ISDGGSYT | 270 | ARDKGPIVPMPM | 271 |
| | | | | | DY | |
| 15B8 | ggattccccttcc | 272 | attcgatttgatt | 273 | gcgagagatgccaacg | 274 |
| | atgatcacggc | | ccacttccaag | | cgagatttggagtaatga | |
| | | | | | tcatggcacat | |
| | GFPFHDHG | 275 | IRFDSTSK | 276 | ARDANARFGVMI | 277 |
| | | | | | MAH | |
| 15A7 | ggatacatgttca | 278 | ataaacccgc | 279 | gcgaggagccagggta | 280 |
| | ccgattatgaa | | acagtggtgac | | ccttcgaggtttactacttt | |
| | | | aca | | gtctcc | |
| | GYMFTDYE | 281 | INPHSGDT | 282 | ARSQGTFEVYYFV | 283 |
| | | | | | S | |
| 5D7 | ggatacaacctc | 284 | atcaacacca | 285 | gcgagagatttcgggga | 286 |
| | actacctatgct | | acactgggaa | | gcggggaaattgtgttaa | |
| | | | ccca | | tggtgtatgctatgggggt | |
| | | | | | tacggt | |
| | | | | | atggacgtc | |
| | GYNLTTYA | 287 | INTNTGNP | 288 | ARDFGERGNCVN | 289 |
| | | | | | GVCYGGYGMDV | |
| 14B3 | ggatacacgttc | 290 | ataaatccgca | 291 | gcgaggagtcagggga | 292 |
| | accgactctgaa | | cagtggtgaca | | ccttcgaggtttactacttt | |
| | | | ca | | ctctcc | |
| | GYTFTDSE | 293 | INPHSGDT | 294 | ARSQGTFEVYYFL | 295 |
| | | | | | S | |
| 2C9 | ggattcagcttca | 296 | atgagtagtagt | 297 | gcgagacggaggtatgg | 298 |
| | gtgacttctac | | ggtggtaacat | | ttcaggaagttcgatctttg | |
| | | | a | | actac | |
| | GFSFSDFY | 299 | MSSSGGNI | 300 | ARRRYGSGSSIFD | 301 |
| | | | | | Y | |
| 2F12 | ggatataccttca | 302 | ataaacccgc | 303 | gcgaggagtcagggaa | 304 |
| | ccgattatgaa | | acagtggtgac | | ccttcgaggtttactacttt | |
| | | | aca | | gtctcc | |
| | GYTFTDYE | 305 | INPHSGDT | 306 | ARSQGTFEVYYFV | 307 |
| | | | | | S | |
| 29B3 | ggatacagcttc | 308 | gtaaacccgc | 309 | gcgaggagtcagggaa | 310 |
| | accgattatgaa | | acagtggtgag | | ccttcgaggtttattactttg | |
| | | | aca | | tctcc | |
| | GYSFTDYE | 311 | VNPHSGET | 312 | ARSQGTFEVYYFV | 313 |
| | | | | | S | |
| 38B7 | ggattcacattca | 314 | atttcgcatgat | 315 | gcgactactcggagtgg | 316 |
| | gaagggatgct | | ggcggtgagg | | ctggtactttgactac | |
| | | | aa | | | |
| | GFTFRRDA | 317 | ISHDGGEE | 318 | ATTRSGWYFDY | 319 |
| 16A8 | ggatataggttca | 320 | ataaatccgca | 321 | gcgaggagccagggta | 322 |
| | ccgattatgaa | | cagtggtgaca | | ccttcgaggtttactacttt | |
| | | | ca | | gtctcc | |
| | GYRFTDYE | 323 | INPHSGDT | 324 | ARSQGTFEVYYFV | 325 |
| | | | | | S | |
| 11F10 | ggattcacctttg | 326 | attcgttggaat | 327 | gtaaaagataacggcct | 328 |
| | atgattatacc | | agtggtaactta | | acggactctagacttc | |
| | GFTFDDYT | 329 | IRWNSGNL | 330 | VKDNGLRTLDF | 331 |
| 32G9 | ggttacacattca | 332 | ataaacccgc | 333 | gcgaggagtcagggtac | 334 |
| | tcgattatgaa | | acagtggtgac | | cttcgaggtctactactttg | |
| | | | aca | | tctcc | |
| | GYTFIDYE | 335 | INPHSGDT | 336 | ARSQGTFEVYYFV | 337 |
| | | | | | S | |
| 24F10 | ggatataccttca | 338 | ataaacccgc | 339 | gcgaggagtcagggaa | 340 |
| | ccgattatgaa | | acagtggtgac | | ccttcgaggtttactacttt | |
| | | | aca | | gtctcc | |
| | GYTFTDYE | 341 | INPHSGDT | 342 | ARSQGTFEVYYFV | 343 |
| | | | | | S | |
| 15A4 | | | | | | |
| 13D9 | ggattcaccttca | 344 | atgagtgcaac | 345 | gcgaggcggaagtttggt | 346 |
| | gtgacttctac | | tggcggtaatat | | gcagggagtgcgatcttt | |
| | | | a | | gaccac | |
| | GFTFSDFY | 347 | MSATGGNI | 348 | ARRKFGAGSAIFD | 349 |
| | | | | | H | |
| 5C5 | ggattcaccttta | 350 | cttacttatagtg | 351 | gcgaaggaggactacg | 352 |
| | gcaaccatgcc | | gtaagaccac | | atgaccggggcttctttga | |
| | | | a | | cttc | |
| | GFTFSNHA | 353 | LTYSGKTT | 354 | AKEDYDDRGFFD | 355 |
| | | | | | F | |
| 14G12 | ggattcaggttca | 356 | gttgcatatgat | 357 | gcgaaacggacccaaa | 358 |
| | gtgactatggc | | gagagcaaga | | aatttgccccctattatttc | |
| | | | aa | | aacggtttggatgtc | |
| | GFRFSDYG | 359 | VAYDESKK | 360 | AKRTQKFAPYYFN | 361 |
| | | | | | GLDV | |
| 17D7 | ggattcaggttca | 362 | gttgcatatgat | 363 | gcgaaacggacccaaa | 364 |
| | gtgactatggc | | gagagcaaga | | aatttgccccctattatttc | |
| | | | aa | | aacggtttggatgtc | |
| | GFRFSDYG | 365 | VAYDESKK | 366 | AKRTQKFAPYYFN | 367 |
| | | | | | GLDV | |
| 3C3 | ggtgcctccatca | 368 | atcgatgctagt | 369 | gcgagacggaagacct | 370 |
| | acagtgatgatta | | gggaccacg | | attacgattttttgactgatt | |
| | ttat | | | | attacaattggtacttcgat | |
| | | | | | gtc | |
| | GASINSDDY | 371 | IDASGTT | 372 | ARRKTYYDFLTDY | 373 |
| | Y | | | | YNWYFDV | |
| 17G11 | ggattcaggttca | 374 | gttgcatatgat | 375 | gcgaaacggacccaaa | 376 |
| | gtgactatggc | | gagagcaaga | | aatttgccccctattatttc | |
| | | | aa | | aacggtttggatgtc | |
| | GFRFSDYG | 377 | VAYDESKK | 378 | AKRTQKFAPYYFN | 379 |
| | | | | | GLDV | |
| 15C4 | ggattcacgttca | 380 | attacgtcaaat | 381 | acgaggtcttacagcag | 382 |
| | gtctgtacacc | | agtagtcacttg | | caaatatgacaactggtt | |
| | | | | | cgacccc | |
| | GFTFSLYT | 383 | ITSNSSHL | 384 | TRSYSSKYDNWF | 385 |
| | | | | | DP | |
| 24C8 | ggattcaggttca | 386 | gttgcatatgat | 387 | gcgaaacggacccaaa | 388 |
| | gtgactatggc | | gagagcaaga | | aatttgccccctattatttc | |
| | | | aa | | aacggtttggatgtc | |
| | GFRFSDYG | 389 | VAYDESKK | 390 | AKRTQKFAPYYFN | 391 |
| | | | | | GLDV | |
| 1A8 | gatgactccatca | 392 | atctattatactg | 393 | gcgagatatcttgattacg | 394 |
| | gtactcctagttac | | ggaccacc | | tttggttgagggcttttgat | |
| | ttc | | | | gtc | |
| | DDSISTPSY | 395 | IYYTGTT | 396 | ARYLDYVWLRAF | 397 |
| | F | | | | DV | |
| 3F9 | ggattcgacttca | 398 | atcagtcgtatt | 399 | gcgagagcaggagcag | 400 |
| | gtagagatact | | gagacttacac | | ttcgtcccggaattggattt | |
| | | | a | | cactactttgacaat | |
| | GFDFSRDT | 401 | ISRIETYT | 402 | ARAGAVRPGIGFH | 403 |
| | | | | | YFDN | |
| 6E10 | | | | | | |
| 4D11 | ggattcaacttta | 404 | attagtagtact | 405 | gcgcgacatttggtcagg | 406 |
| | ctaactactac | | actaatagcat | | gggacctctcttgctgcttt | |
| | | | a | | tgatatc | |
| | GFNFTNYY | 407 | ISSTTNSI | 408 | ARHLVRGTSLAAF | 409 |
| | | | | | DI | |
| 1H9 | ggatacaccttca | 410 | attaacacgaa | 411 | gcgacagatggtagtga | 412 |
| | ctaagtatggt | | cactgcaaag | | gggctcc | |
| | | | cca | | | |
| | GYTFTKYG | 413 | INTNTAKP | 414 | ATDGSEGS | 415 |
| 7B6 | ggtgggtccttca | 416 | atcgatcatagt | 417 | gcgaggaggcgcattgg | 418 |
| | atggttactac | | ggaagcacc | | gagcctattaaaatacttc | |
| | | | | | caggac | |
| | GGSFNGYY | 419 | IDHSGST | 420 | ARRRIGSLLKYFQ | 421 |
| | | | | | D | |
| 8F3 | gatgcctccatcg | 422 | atctattatactg | 423 | gcgagatatgttgattatg | 424 |
| | acactccgagtta | | ggaacaag | | tttggttgagggcttttgat | |
| | cttc | | | | ata | |
| | DASIDTPSY | 425 | IYYTGNK | 426 | ARYVDYVWLRAF | 427 |
| | F | | | | DI | |
| 9A11 | ggattcaccttca | 428 | ctgaataaaga | 429 | gtcagagatcgacgagc | 430 |
| | atacttactgg | | tggcagtagtc | | ggtgactacggcccggt | |
| | | | ca | | acttcgatctc | |
| | GFTFNTYW | 431 | LNKDGSSP | 432 | VRDRRAVTTARYF | 433 |
| | | | | | DL | |
| 15C2 | ggatacgacctc | 434 | atcaacacca | 435 | tcgagagatggtagtgg | 436 |
| | actaaatatgct | | acactgcgaa | | ggcctcc | |
| | | | acca | | | |
| | GYDLTKYA | 437 | INTNTAKP | 438 | SRDGSGAS | 439 |
| 20M10 | ggattcaccttca | 440 | attagtagtact | 441 | gtgagacgaggtcgggg | 442 |
| | atatgcatagc | | agcacttacat | | tggagcagcccgtgcac | |
| | | | C | | ttgacaac | |
| | GFTFNMHS | 443 | ISSTSTYI | 444 | VRRGRGGAARAL | 445 |
| | | | | | DN | |
| 21C10 | ggattcattctca | 446 | attagtagtact | 447 | gcgagaaaaaccaatg | 448 |
| | gtaggagtagc | | agtagtcatata | | gggcctggttcctcgatct | |
| | | | | | C | |
| | GFILSRSS | 449 | ISSTSSHI | 450 | ARKTNGAWFLDL | 451 |
| 22F2 | ggatacgacctc | 452 | atcaacacca | 453 | tcgagagatggtagtgg | 454 |
| | actaaatatgct | | acactgcgaa | | ggcctcc | |
| | | | acca | | | |
| | GYDLTKYA | 455 | INTNTAKP | 456 | SRDGSGAS | 457 |
| 26A5 | ggatacaccttca | 458 | attaacacgaa | 459 | gcgacagatggtagtga | 460 |
| | ctaagtatggt | | cactgcaaag | | gggctcc | |
| | | | cca | | | |
| | GYTFTKYG | 461 | INTNTAKP | 462 | ATDGSEGS | 463 |
| 3B7 | ggatacaccttca | 464 | atcaacacca | 465 | gcgagaggcgagtccct | 466 |
| | ctaactatgct | | acactgggaa | | agcagccctcggttcgttc | |
| | | | acca | | gcctac | |
| | GYTFTNYA | 467 | INTNTGKP | 468 | ARGESLAALGSFA | 469 |
| | | | | | Y | |
| 7E6 | ttttttaactttaga | 470 | attagtggaact | 471 | gcgcaactgcatctcggt | 472 |
| | atgtatccc | | ggtcagacca | | tattatcatgagagcagtg | |
| | | | ca | | gttattttttaagt | |
| | FFNFRMYP | 478 | ISGTGQTT | 479 | AQLHLGYYHESS | 480 |
| | | | | | GYFLS | |
| 14C11 | ggtggctccatca | 481 | ctcttttccggtg | 482 | gcgagacaacgggcag | 483 |
| | gcagtaggacttt | | ggaccacc | | cattaccgccctactacta | |
| | cttc | | | | ctactacttcgacgtc | |
| | GGSISSRTF | 484 | LFSGGTT | 485 | ARQRAALPPYYYY | 486 |
| | F | | | | YFDV | |
| 31A12 | ggattcaggttca | 487 | attactagtagc | 488 | gcgagagatgggggttg | 489 |
| | atgagcatagt | | ggaaataccat | | taatggtcgcacctgcta | |
| | | | a | | cggactcaactac | |
| | GFRFNEHS | 490 | ITSSGNTI | 49 | ARDGGCNGRTCY | 492 |
| | | | | | GLNY | |
| 20G11 | ggattcaccttca | 493 | atacggtataat | 494 | gcgaaagacggcgatta | 495 |
| | gtagttatggc | | ggaagtaataa | | cgattcttggagtggtctc | |
| | | | a | | actgaacacttccagcac | |
| | GFTFSSYG | 496 | IRYNGSNK | 497 | AKDGDYDSWSGL | 498 |
| | | | | | TEHFQH | |
| 9H11 | ggattcgacttcc | 499 | atatggtacgat | 500 | gcggtgggagctcctctc | 501 |
| | ctggctatggc | | gccagaagtg | | gaagggtac | |
| | | | aa | | | |
| | GFDFPGYG | 502 | IWYDARSE | 503 | AVGAPLEGY | 504 |
| 26C3 | ggtggctccatca | 505 | atctttaccact | 506 | gcgagactgaggagggt | 507 |
| | gtagttactac | | gggagcacc | | agtaccaactgctatctg | |
| | | | | | gcacttcgatctc | |
| | GGSISSYY | 508 | IFTTGST | 509 | ARLRRVVPTAIWH | 510 |
| | | | | | FDL | |

TABLE 4
CDR LIGHT CHAIN SEQUENCES

| | | (SEQ ID | | | (SEQ ID |
|---|---|---|---|---|---|
| Clone | CDRL1 | NO: ) | CDRL2 | CDRL3 | NO: ) |
| 40C7 | cagagtgttttagacagct | 511 | tgggcatct | cagcaatattatagtactc | 512 |
| | ccaacaataagaagtac | | | ctccgacg | |
| | QSVLDSSNNKKY | 513 | WAS | QQYYSTPPT | 514 |
| 3B10 | gagaatatttataagtgg | 515 | aaggtgtct | caacattataacagtttcc | 516 |
| | | | | ccttcact | |
| | ENIYKW | 517 | KVS | QHYNSFPFT | 518 |
| 4G4 | cagagtgttttagacagct | 519 | tgggcatct | cagcaatattatactactc | 520 |
| | ccaacaataagaagtac | | | ctccgacg | |
| | QSVLDSSNNKKY | 521 | WAS | QQYYTTPPT | 522 |
| 16G12 | aaattgggggccaaattt | 523 | caagatacc | caggcgtgggaaagtgg | 524 |
| | | | | tattgtggcg | |
| | KLGAKF | 525 | QDT | QAWESGIVA | 526 |
| 15B8 | caggacattggacactat | 527 | gatgcatcc | catcagcatgagaagctt | 528 |
| | | | | tactcgatctcc | |
| | QDIGHY | 529 | DAS | HQHEKLYSIS | 530 |
| 15A7 | aacattgcaacaaaacg | 531 | gatgaggag | caggtgtgggatagtctg | 532 |
| | t | | | gctgaccaagtggta | |
| | NIATKR | 533 | DEE | QVWDSLADQVV | 534 |
| 5D7 | cagagtattagccggtgg | 535 | aaggcgtct | caagaatatactaattatt | 536 |
| | | | | cggggacg | |
| | QSISRW | 537 | KAS | QEYTNYSGT | 538 |
| 14B3 | | | | | |
| 2C9 | acattgggtgatagatat | 539 | caaagtggc | ctgacgtgggaccgcgg | 540 |
| | | | | cactcctgtc | |
| | TLGDRY | 541 | QSG | LTWDRGTPV | 542 |
| 2F12 | ggaattggcaggaaacg | 543 | gatgatgac | caggtgtgggatagtag | 544 |
| | t | | | gactgaccaagtggtg | |
| | GIGRKR | 545 | DDD | QVWDSRTDQVV | 546 |
| 29B3 | | | | | |
| 38B7 | cagagtgttgacacctac | 547 | gatgcatcc | cagcagcgtaacaactg | 548 |
| | | | | gcggacgtggacg | |
| | QSVDTY | 549 | DAS | QQRNNWRTWT | 550 |
| 16A8 | aacattgccaagaaacg | 551 | gatgatgag | caggtgtgggatagtaa | 552 |
| | t | | | gactgaccacgtggtt | |
| | NIAKKR | 553 | DDE | QVWDSKTDHVV | 554 |
| 11F10 | cagagcctcctccatagg | 555 | ttggcttct | atgcaagctctacaaact | 556 |
| | aatggatacaactat | | | tggacg | |
| | QSLLHRNGYNY | 557 | LAS | MQALQTWT | 558 |
| 32G9 | aacattgtaagaaaacgt | 559 | gatgatgag | caggtgtgggatagtacg | 560 |
| | | | | actgaccaggtggta | |
| | NIVRKR | 561 | DDE | QVWDSTTDQVV | 562 |
| 24F10 | | | | | |
| 15A4 | cagaatattttagacaact | 563 | tggggttct | ctccaatattatagtcttcc | 564 |
| | ccaacaataagaacttc | | | tcacact | |
| | QNILDNSNNKNF | 565 | WGS | LQYYSLPHT | 566 |
| 13D9 | aaattgggtgaaagatat | 567 | caaactaac | ctgacgtgggaccgcgg | 568 |
| | | | | cactcctgtc | |
| | KLGERY | 569 | QTN | LTWDRGTPV | 570 |
| 5C5 | cagaccattagtacttat | 571 | gctgcatcc | caacagggttacaataa | 572 |
| | | | | cccgtacact | |
| | QTISTY | 573 | AAS | QQGYNNPYT | 574 |
| 14G12 | cagagcctcctggatcgt | 575 | ttgggttct | atgcaagctctacaaatt | 576 |
| | aatggatacaactac | | | ccgatcacc | |
| | QSLLDRNGYNY | 577 | LGS | MQALQIPIT | 578 |
| 17D7 | cagagcctcctggatcgt | 579 | ttgggttct | atgcaagctctacaaatt | 580 |
| | aatggatacaactac | | | ccgatcacc | |
| | QSLLDRNGYNY | 581 | LGS | MQALQIPIT | 582 |
| 3C3 | aatagtgacattcgtgatt | 583 | gatgtcact | tgttcgtattcagtcactta | 584 |
| | atgattat | | | ctctttcgaggtc | |
| | NSDIRDYDY | 585 | DVT | CSYSVTYSFEV | 586 |
| 17G11 | cagagcctcctggatcgt | 587 | ttgggttct | atgcaagctctacaaatt | 588 |
| | aatggatacaactac | | | ccgatcacc | |
| | QSLLDRNGYNY | 589 | LGS | MQALQIPIT | 590 |
| 15C4 | aacttgggggataagtat | 591 | cgagataat | caggcgttggacagtgg | 592 |
| | | | | cagtttttgggtg | |
| | NLGDKY | 593 | RDN | QALDSGSFWV | 594 |
| 24C8 | cagagcctcctggatcgt | 595 | ttgggttct | atgcaagctctacaaatt | 596 |
| | aatggatacaactac | | | ccgatcacc | |
| | QSLLDRNGYNY | 597 | LGS | MQALQIPIT | 598 |
| 1A8 | ccgagtgttggcaggttc | 599 | gatgcatct | cagcaccgtagcaactg | 600 |
| | | | | gccggtcact | |
| | PSVGRF | 601 | DAS | QHRSNWPVT | 602 |
| 3F9 | | | | | |
| 6E10 | cagagcattggtcgctgg | 603 | agggcctct | caacaatataatactaatt | 604 |
| | | | | cggggaca | |
| | QSIGRW | 605 | RAS | QQYNTNSGT | 606 |
| 4D11 | | | | | |
| 1H9 | cagagtattggtacctgg | 607 | aaggcgtct | caacaatataatacttact | 608 |
| | | | | cggggacg | |
| | QSIGTW | | KAS | QQYNTYSGT | 610 |
| 7B6 | aaattgggggatagttat | 611 | caagatacc | caggcgtgggacacca | 612 |
| | | | | ccacggattgggtg | |
| | KLGDSY | 613 | QDT | QAWDTTTDWV | 614 |
| 8F3 | ccgagtgctggccgcttc | 615 | gatgcatcc | caacaccgtagcaactg | 616 |
| | | | | gccgctcact | |
| | PSAGRF | 617 | DAS | QHRSNWPLT | 618 |
| 9A11 | tccaacattgataactac | 619 | tgggcatct | caacaatattatactagtc | 620 |
| | | | | ctccggtcact | |
| | SNIDNY | 621 | WAS | QQYYTSPPVT | 622 |
| 15C2 | cagaatattggtcgctgg | 623 | aaggcgtcc | cagcaatatactacttatt | 624 |
| | | | | cggggacg | |
| | QNIGRW | 625 | KAS | QQYTTYSGT | 626 |
| 20M10 | aacactggaagtaaaag | 627 | gataatagc | caggtgtgggatagtact | 628 |
| | t | | | attgatcatgtgata | |
| | NTGSKS | 629 | DNS | QVWDSTIDHVI | 630 |
| 21C10 | agctccaacatcggggc | 631 | ggaaataac | cagtcctatgactacgac | 632 |
| | aggttctgat | | | ctgagtgggtcttgggtc | |
| | SSNIGAGSD | 633 | GNN | QSYDYDLSGSWV | 634 |
| 22F2 | | | | | |
| 26A5 | cagagtattggtacctgg | 635 | aaggcgtct | caacaatataatacttact | 636 |
| | | | | cggggacg | |
| | QSIGTW | 637 | KAS | QQYNTYSGT | 638 |
| 3B7 | cagagcattaacagctat | 639 | gctgcatcc | caacagagttccagtact | 640 |
| | | | | ttttggacg | |
| | QSINSY | 641 | AAS | QQSSSTFWT | 642 |
| 7E6 | cagactataagtagttgg | 643 | aaggcgtct | caacagtatactagttctt | 644 |
| | | | | ggacg | |
| | QTISSW | 645 | KAS | QQYTSSWT | 646 |
| 14C11 | cagagtgttggcagatac | 647 | gatgcatcc | cagcagcgtggcaactg | 648 |
| | | | | gcctccgatcacc | |
| | QSVGRY | 649 | DAS | QQRGNWPPIT | 650 |
| 31A12 | agatccgacattgggaat | 651 | gacactttt | ggaacatgggatatcag | 652 |
| | agtttt | | | cctgagtgcggcggtc | |
| | RSDIGNSF | 653 | DTF | GTWDISLSAAV | 654 |
| 20G11 | cagaatgttttatacagct | 655 | tgggcatct | cagcaatattatagtatac | 656 |
| | ccaacaataagaactac | | | ctccggct | |
| | QNVLYSSNNKNY | 657 | WAS | QQYYSIPPA | 658 |
| 9H11 | cagaacattgccgactat | 659 | gctgcatct | cagcagagttacacttca | 660 |
| | | | | cccacgtggacg | |
| | QNIADY | 661 | AAS | QQSYTSPTWT | 662 |
| 26C3 | tctggcgcagtctctacta | 663 | ggcacaaac | gtgttgtatatggatagtg | 664 |
| | gttacttc | | | gcgtgatggta | |
| | SGAVSTSYF | 665 | GTN | VLYMDSGVMV | 666 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined herein.

### VII. REFERENCES

The following references, to the extent that they provide exemplary procedural or other details supplementary to those set forth herein, are specifically incorporated herein by reference.
“Antibodies: A Laboratory Manual,” Cold Spring Harbor Press, Cold Spring Harbor, N Y, 1988.
Abbondanzo et al., Am. J. Pediatr. Hematol. Oncol., 12(4), 480-489, 1990.
Abraham et al., Infect Immun.; 72:810-7, 2004.
Acevedo et al., Allergy, 64(11): 1635-43, 2009.
Achatz et al., Science, 276(5311):409-11, 1997.
Allred et al., Arch. Surg., 125(1), 107-113, 1990.
American Academy of Allergy Asthma and Immunology: Food allergy. World-wide-web at aaaai.org/conditions-and-treatments/allergies/food-allergies.aspx
Asthma and Allergy Foundation of America: Allergy facts and figures. World-wide-web at aafa.org/display.cfm?id=9&sub=30
Atherton et al., Biol. of Reproduction, 32, 155-171, 1985.
Avery et al., Blood; 112(5):1784-93, 2008.
Brown et al., J. Immunol. Meth., 12; 130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Chapa-Ruiz et al., Parasite; 8:S163-7, 2001.
Cooper et al., J Allergy Clin Immunol., 111:995-1000, 2003.
Cruz et al., Clin Exp Allergy; 37:197-207, 2007.
De Jager et al., Semin. Nucl. Med. 23(2), 165-179, 1993.
Dholakia et al., J. Biol. Chem., 264, 20638-20642, 1989.
Doolittle and Ben-Zeev, Methods Mol. Biol., 109:215-237, 1999.
Emsley & Cowtan, Acta Cryst. 60(Pt 12 Pt 1):2126-32, 2004.
Finkelman, et al., Immunol. Rev.; 201:139-155, 2004.
Food Allergy Among U.S. Children: Trends in Prevalence and Hospitalizations. world-wide-web at cdc.gov/nchs/data/databriefs/db10.htm
Gefter et al., Somatic Cell Genet., 3:231-236, 1977.
Gulbis and Galand, Hum. Pathol. 24(12), 1271-1285, 1993.
Gurish et al., J Immunol.; 172:1139-45, 2004.
Hagan et al., Nature, 349:243-245, 1991.
Hagel et al., Acta Trop., 103:231-241, 2007.
Herbert et al., Immunity; 20:623-635, 2004.
Johansson and Bennich, Immunology; 13(4):381-94, 1967.
Karnowski et al., Eur J Immunol, 36(7): 1917-25, 2006.
Khatoon et al., Ann. of Neurology, 26, 210-219, 1989.
King et al., J Immunol; 158:294-300, 1997.
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Kyte and Doolittle, J. Mol. Biol., 157(1): 105-132, 1982.
Lee et al., J Infect Dis., 162:529-33, 1990.
Leonardi-Bee et al., Am J Respir Crit Care Med., 174:514-523, 2006.
Liu and Leung, J Allergy Clin Immunol., 117:1063-1066, 2006.
Lobos et al., Mol Med., 2(6):712-24, 1996.
Mccarthy et al., J Infect Dis., 170:736-41, 1994.
McSharry et al., Infect Immun; 67:484-489, 1999.
Mitre et al., J Immunol; 172:2439-45, 2004.
Mitre and Nutman, J Allergy Clin Immunol., 117(4):939-45, 2006.
Nakamura et al., In: Enzyme Immunoassays: Heterogeneous and Homogeneous Systems, Chapter 27, 1987.
Nutman et al., J. Infect. Dis.; 160:1042-1050, 1989.
Olsson and Kaplan, Proc Natl Acad Sci USA; 77(9):5429-31, 1980.
O'Shannessy et al., J. Immun. Meth., 99, 153-161, 1987.
Persic et al., Gene 187:1, 1997.
Potter and Haley, Meth. Enzymol., 91, 613-633, 1983.
Remington's Pharmaceutical Sciences, 15th Ed., 3:624-652, 1990.
Renz et al., J Immunol.; 145(11):3641-6, 1990.
Rodrigues et al., Clin Exp Allergy; 38:1769-1777, 2008.
Santiago et al., J Immunol., 194(1):93-100, 2015.
Santiago et al., J Allergy Clin Immunol., 130(1):248-56, 2012.
Santiago et al., PLOS One, 7(7):e40552, 2012.
Sicherer et al., J Allergy Clin Immunol., April; 103(4):559-62, 1999.
Steel, J Infect Dis., 164:581-7, 1991.
Strachan, BMJ, 299:1259-1260, 1989.
Tang et al., J. Biol. Chem., 271:28324-28330, 1996.
Turner et al., J Infect Dis., 188:1768-1775, 2003.
Turner et al., Microbes Infect., 7:990-996, 2005.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
van den Biggelaar et al., Lancet.; 356:1723-1727, 2000.
Voehringer et al., J. Exp. Med., 203:1435-1446, 2006.
Wawrzynczak & Thorpe, In: Immunoconjugates, Antibody Conuugates In Radioimaging And Therapy Of Cancer, Vogel (Ed.), NY, Oxford University Press, 28, 1987.
Wordemann et al., Trop Med Int Health.; 13:180-186, 2008.
Beck L A, Marcotte G V, MacGlashan D, Togias A, Saini S. Omalizumab-induced reductions in mast cell Fc epsilon RI expression and function. J Allergy Clin Immunol. 2004; 114(3):527-530.
Chen X, Negi S S, Liao S, Gao V, Braun W, Dreskin S C. Conformational IgE epitopes of peanut allergens Ara h 2 and Ara h 6. Clin Exp Allergy. 2016; 46(8): 1120-1128.
Dirks C G, Pedersen M H, Platzer M H, Bindslev-Jensen C, Skov P S, Poulsen L K. Does absorption across the buccal mucosa explain early onset of food-induced allergic systemic reactions? J Allergy Clin Immunol. 2005; 115(6):1321-1323.
Dombrowicz D, Brini A T, Flamand V, Hicks E, Snouwaert J N, Kinet J P, Koller B H. Anaphylaxis mediated through a humanized high affinity IgE receptor. J Immunol. 1996; 157(4):1645-1651.
Mueller G A, Gosavi R A, Pomés A, Wünschmann S, Moon A F, London R E, Pedersen L C. Ara h 2: crystal structure and IgE binding distinguish two subpopulations of peanut allergic patients by epitope diversity. Allergy. 2011; 66(7):878-885.
Osterfeld H, Ahrens R, Strait R, Finkelma F D, Renauld J C, and Hogan S P. Differential roles for IL-9/IL-9 receptor α-chain pathway in systemic and oral antigen-induced anaphylaxis. J Allergy Clin Immunol. 2010; 125:496-476.
Smith K, Garman L, Wrammert J, Zheng N Y, Capra J D, Ahmed R, Wilson P C. Rapid generation of fully human monoclonal antibodies specific to a vaccinating antigen. Nat Protoc. 2009; 4(3):372-384.
Smrz D, Cruse G, Beaven M A, Kirshenbaum A, Metcalfe D D, and Gilfillan A M. Rictor negatively regulates high-affinity receptors for IgE-induced mast cell degranulation. J Immunol. 2014; 193:(12)5924-5932.
Shreffler W G, Beyer K, Chu T H, Burks A W, Sampson H A. Microarray immunoassay: association of clinical history, in vitro IgE function, and heterogeneity of allergenic peanut epitopes. J Allergy Clin Immunol. 2004; 113(4):776-82.
Wurth M A, Hadadianpour A, Horvath D J, Daniel J, Bogdan O, Goleniewska K, Pomés A, Hamilton R G, Peebles R S Jr, Smith S A. Human IgE mAbs define variability in commercial Aspergillus extract allergen composition. JCI Insight. 2018; 3(20): e123387.

1. A method of detecting a IgE antibody with binding affinity/specificity for a peanut antigen in a subject comprising:
(a) providing a test antibody or fragment thereof antibody or antibody fragment characterized by clone paired heavy and light chain CDRs from Tables 3 and 4;
(b) contacting the test antibody or fragment thereof with an antibody-containing sample from said subject in the presence of a peanut antigen; and
(c) detecting IgE antibody with binding affinity for peanut antigen in said sample by measuring the reduction of binding to peanut antigen by the test antibody or fragment thereof as compared to the binding of the test antibody or fragment thereof in the absence of said sample.

2.-12. (canceled)

13. A method of detecting a peanut allergen or antigen in a sample comprising:
(a) providing a test antibody or fragment thereof antibody or antibody fragment characterized by clone paired heavy and light chain CDRs from Tables 3 and 4;
(b) contacting the test antibody or fragment thereof with a sample suspect of containing a peanut allergen or antigen; and
(c) detecting a peanut allergen or antigen in said sample by binding of the test antibody or fragment.

14.-22. (canceled)

23. A method of preventing or treating a peanut-related allergic reaction in a subject comprising delivering to said subject an IgG antibody or antibody fragment, wherein said antibody or antibody fragment is characterized by clone paired heavy and light chain CDRs from Tables 3 and 4.

24. The method of claim 23, wherein the antibody or antibody fragment is encoded by heavy and light chain variable sequences as set forth in Table 1.

25. The method of claim 23, wherein said antibody or antibody fragment is encoded by heavy and light chain variable sequences having 70%, 80%, 90% or 95% identity to heavy and light chain variable sequences as set forth in Table 1.

26. (canceled)

27. The method of claim 23, wherein said antibody or antibody fragment comprises heavy and light chain variable sequences as set forth in Table 2.

28. The method of claim 23, wherein said antibody or antibody fragment comprises heavy and light chain variable sequences having 70%, 80% 90% or 95% identity to heavy and light chain variable sequences as set forth in Table 2.

29. (canceled)

30. The method of claim 23, wherein said antibody fragment is a recombinant scFv (single chain fragment variable) antibody, Fab fragment, F(ab′)₂ fragment, or Fv fragment, a chimeric antibody or a bispecific antibody.

31. The method of claim 23, further comprising treating said subject with an anti-inflammatory agent.

32. (canceled)

33. (canceled)

34. The method of claim 23, wherein delivering comprises antibody or antibody fragment administration.

35. The method of claim 32, wherein delivering comprises genetic delivery with an RNA or DNA sequence or vector encoding the antibody or antibody fragment.

36. A monoclonal antibody or antibody fragment comprises clone paired heavy and light chain CDRs from Tables 3 and 4.

37.-45. (canceled)

46. A hybridoma or engineered cell encoding an antibody or antibody fragment wherein the antibody or antibody fragment is characterized by clone paired heavy and light chain CDRs from Tables 3 and 4.

47.-55. (canceled)

56. A vaccine formulation comprising one or more IgG antibodies or antibody fragments characterized by clone paired heavy and light chain CDRs from Tables 3 and 4.

57.-64. (canceled)

65. A method of de-sensitizing a subject to a peanut allergen comprising:
(a) administering to said subject a peanut allergen; and
(b) administering to said subject an IgG antibody or antibody fragment characterized by clone paired heavy and light chain CDRs from Tables 3 and 4.

66.-68. (canceled)

69. The method of claim 65, wherein said subject is a human or a non-human mammal.

70. The method of claim 65, wherein said peanut allergen is administered with an adjuvant.

71. A method of producing an IgG immune response to a peanut allergen comprising:
(a) identifying an IgE epitope in an allergen by mapping the binding of an IgE antibody binding site;
(b) modifying one or more residues in said IgE antibody binding site to reduce or eliminate IgE antibody binding to said binding site, thereby producing a hypoallergenic allergen;
(c) immunizing a subject with said hypoallergenic allergen to produce and IgG response to said hypoallergenic allergen, while producing a reduced or no IgE response as compared to the allergen of step (a).

72. The method of claim 71, wherein IgE antibody binding to said binding site is reduced by at least 50%, at least 90% or 100%.

73.-75. (canceled)

76. A method of determining the antigenic integrity of a peanut antigen comprising:
(a) contacting a sample comprising said peanut antigen with a first antibody or antibody fragment having clone-paired heavy and light chain CDR sequences from Tables 3 and 4, respectively; and
(b) determining antigenic integrity of said peanut antigen by detectable binding of said antibody or antibody fragment to said antigen.

77.-96. (canceled)


SEQUENCE LISTING (666 sequences)

SEQ ID NO: 1 - DNA, 364 bp, Artificial sequence
  Synthetic antibody
gaggtgcagctggtggagtcggggggagacttggtacagccgggggggtccctgagactctcttgtgcagcctctagattcagctttggcagctatgccatgagttgggtccgccaggctccagggaagggcctggagtgggtctcagcgatttcttctagtggtggtagggcatactacgcagactccgtgaagggccggttcaccatctccagagacaattccaggaacaccctgtatctgcagttgaacagcctgagaggcgaggacacggccttctattactgtacgaaagaaggtgacacagggtccctatggttatttgactcctggggccagggaaccctggtcaccgtctcctcag

SEQ ID NO: 2 - DNA, 340 bp, Artificial sequence
  Synthetic antibody
gacatcgtgatgacccagtctccagactccctggctgtgtctctgggcgagagggccaccatcaactgcaagtccagccagagtgttttagacagctccaacaataagaagtacttagcttggtaccagcagaaaccaggacagcctcctaagctgctcatttactgggcatctactcgggaattcggggtccctgaccgattcagtggcagcgggtctgggacagatttcactctcaccatcagcagcctgcaggctgaagatgtggcagtttattactgtcagcaatattatagtactcctccgacgttcggccaagggaccaaggtggaaatcaaac

SEQ ID NO: 3 - DNA, 370 bp, Artificial sequence
  Synthetic antibody
gaggtgcagctggtggagtctgggggagccctggtaaagccgggggggtctcttcgactctcctgtgaaggctctggtttcatgttcagtagcgcctggttgcactgggtccggcaggctccagggatgggactggaattggtcggccgtatgaaaagtgagactgatggtgggacacttgactacactgcacccgtgagaggcagattcaccatctcaagagatgattcaaaaaatatgttttttctgcaaatgaatagcctgaaagccgaggacaccgccgtctattattgtgtcacaggaaacttcgattataggagttcaccccttggcttctggggccagggagccctggtcaccgtctcctcag

SEQ ID NO: 4 - DNA, 322 bp, Artificial sequence
  Synthetic antibody
gacatccagatgacccagtctccttctactctgtctgcgtctgcaggagacagagtcaccatcacttgccgggccagtgagaatatttataagtggttggcctggtatcagcagaaaccagggaaagcccctaaattgctgatctacaaggtgtctactttagaaagtggggtcccatcacggttcagcggcagtggatttgggacagaattcactctcaccatcagcagcctgcagcctggtgattttgcgacttattactgccaacattataacagtttccccttcacttttggccaggggaccaagctggaggtcaaac

SEQ ID NO: 5 - DNA, 364 bp, Artificial sequence
  Synthetic antibody
gaggtgcagctggtgcagtcggggggaggcttggtacagcctggggggtccctgagactctcctgtgcagcctctagattcatctttggcagctatgccatgagttgggtccgccaggctccagggaaggggctggagtgggtctcagccattagttctagtggtggcaggacatactacgcagactccgtgaagggccggttcaccatctccagagacaattccaggaacacgctgtacctgcagttgagcagcctgagagccgaggacacggccttctattactgtacgaaagaaggtgacacagggtccctatggctttttgactcctggggccagggaaccctggtcaccgtctcctcag

SEQ ID NO: 6 - DNA, 340 bp, Artificial sequence
  Synthetic antibody
gacatcgtgatgacccagtctccagactccctggctgtgtctctgggcgagagggccaccatcaactgcaagtccagccagagtgttttagacagctccaacaataagaagtacttagcttggtaccagcagaaaccaggacagtctcctaagctgctcatttactgggcatctactcgggaattcggggtccctgaccgattcagtggcagcgggtctgggacagatttcactctcaccatcagcagcctgcaggctgaagatgtggcagtttattactgtcagcaatattatactactcctccgacgttcggccgagggaccaaggtggaaatcaaac

SEQ ID NO: 7 - DNA, 364 bp, Artificial sequence
  Synthetic antibody
gaggtgcagctggtggagtctgggggaggcttagtgaagcctggagggtccctgaaactctcctgtgcagcctctggattcactttcagtgactattacatgtattgggttcgccagactccggaaaagaggctggagtgggtcgcaaccattagtgatggtggtagttacacctactatccagacaatgtaaagggccgattcaccatctccagagacaatgccaagaacaccctgtacctgcaaatgagccatctgaagtctgaggacacagccatgtattactgtgcaagagataaagggcctatagtccctatgcctatggactactggggtcaaggaacctcagtcaccgtctcctcag

SEQ ID NO: 8 - DNA, 319 bp, Artificial sequence
  Synthetic antibody
tcctatgagctgactcagccactctcagtgtccgtgtccccaggacagactgtcaacatcacctgctctggagataaattgggggccaaatttacctgctggtatcagcagaagccaggccagtcccctatcctagtcctttatcaagataccaagcggcccccagggatccctgagcgattctctggctccaactctgggaacacagccactctgaccatcagcgggacccaggctatggatgaggctgactattactgtcaggcgtgggaaagtggtattgtggcgttcggcggagggaccaagctgaccgtcgtag

SEQ ID NO: 9 - DNA, 367 bp, Artificial sequence
  Synthetic antibody
caggtgcagctggtggagtctgggggacgcgtggtccagccgggggagtccctgagactctcctgtgaaacatctggattccccttccatgatcacggcatgcactgggtccggcaggctccaggcaagggactggagtgggtggcatttattcgatttgattccacttccaagtatacttcagactccgtgaggggccgattcagcatttccagagacaattcaaagagcacactatttctccaaatgaacaacctgcgacgtgaggacacggctatatattactgtgcgagagatgccaacgcgagatttggagtaatgatcatggcacattggggccagggaaccctggtcaccgtctcctcag

SEQ ID NO: 10 - DNA, 325 bp, Artificial sequence
  Synthetic antibody
gacatccagttgacccagtctccatcctccctgtctgcatctgtcggagacagagtcaccatcacttgtcaggcgagtcaggacattggacactatttaaattggtatctgcaaaaaccaggtcaagccccgcaggtcctgatttacgatgcatccaatttggtaacaggggtcccatcaagattcagtggaagtggatctgggacacattttactttcaccatcagcagcctgcagcctgaagattttggaacatactattgtcatcagcatgagaagctttactcgatctccttcggccaagggacacgactggacattaaac

SEQ ID NO: 11 - DNA, 364 bp, Artificial sequence
  Synthetic antibody
caggtgcagctggtgcagtctgggcctgaagtgaggaagcctggggcctcagtgaaggtctcctgtaagacttctggatacatgttcaccgattatgaaatgaattgggtgcgacaggcccctggacagggccttgagtggatgggactcataaacccgcacagtggtgacacaggctacgcacagaagttccagggcagagttagcatgaccagcgacacctccacaagaactttctacttggagctgactggcctgacatctgaggacacggccgtgtattactgtgcgaggagccagggtaccttcgaggtttactactttgtctcctggggccagggatccctagtcaccgtctcctcag

SEQ ID NO: 12 - DNA, 325 bp, Artificial sequence
  Synthetic antibody
tcctatgtgctgactcagccaccctcggtgtcagtggccccagggaagacggccagcattacctgtggggcaaagaacattgcaacaaaacgtgtacactggtaccggcagaagccaggccaggcccctgtggtggtcgtctctgatgaggaggatcgatcctcagtgatccctgagcgattctctggctccaaatctggggacacggccaccctggaaatcagcagggtcgaggccggggatgaggccgattattactgtcaggtgtgggatagtctggctgaccaagtggtattcggcggagggaccaaggtgaccgtcctgg

SEQ ID NO: 13 - DNA, 390 bp, Artificial sequence
  Synthetic antibody
caggtgcagttggtgcaatctgggtctgagttgaagaagcctggggcctcagtgaaggtttcctgcgaggcctctggatacaacctcactacctatgctatgaattgggtgcgacaggcccctggacaagggcttgagtggatgggatggatcaacaccaacactgggaacccaacgtatgcccaggacttcacaggacactttgtcttctccctggacacctctgtcagtacggcatatctgcagatcagcagcctaaaggctgaggacactgccgtgtattactgtgcgagagatttcggggagcggggaaattgtgttaatggtgtatgctatgggggttacggtatggacgtctggggccaagggaccacggtcaccgtctcctca

SEQ ID NO: 14 - DNA, 322 bp, Artificial sequence
  Synthetic antibody
gacatccagatgacccagtctccttccaccctgtctgcatctgtaggagacagagtcaccatcacttgccgggccagtcagagtattagccggtggttggcctggtatcagcagaaaccagggaaagtccctaagctcctgatctataaggcgtctagattagaaggtggggtcccatcaaggttcagcggcagtggatctgggacagaattcactctcaccatcagcagcctgcagcctgatgattttgcaacttattactgccaagaatatactaattattcggggacgttcggccaagggaccaaggtggaaatcagac

SEQ ID NO: 15 - DNA, 364 bp, Artificial sequence
  Synthetic antibody
caggtgcagctggtgcagtctgggcctgaagtgaagaagcctggggcctcagtgaaggtctcctgtaagacttctggatacacgttcaccgactctgaaatgaactgggtgcgacaggcccctggacagggccttgagtggatgggactcataaatccgcacagtggtgacacacgctatgcagagaggttccagggcagagtcaccatgaccagcgacacctccataaacacagtctacttggagttgagtggcctgacatctgaggacacggccatatatttctgtgcgaggagtcaggggaccttcgaggtttactactttctctcctggggccagggatcgctaatcaccgtctcctcag

SEQ ID NO: 16 - skipped

SEQ ID NO: 17 - DNA, 364 bp, Artificial sequence
  Synthetic antibody
caggtgcagctggtggagtctgggggaggcttggtcaagcctggagggtccctgagactctcctgtgcagcctctggattcagcttcagtgacttctacatgagctggatacgccaggctccaggcaagggactggagtgtatttcttatatgagtagtagtggtggtaacatatactatgcagactctgtgaagggccgattcaccatctccagggacaacgccaagaattcactgtctctgcaaatgaacagcctgagagccgacgacacggccctctattattgtgcgagacggaggtatggttcaggaagttcgatctttgactactggagccagggaaccctggtcaccgtctcctcag

SEQ ID NO: 18 - DNA, 319 bp, Artificial sequence
  Synthetic antibody
tcctatgagctgactcagccaccctcagtgtccgtgtccccaggacagacagccagcgtcacctgctcaggagatacattgggtgatagatatgtgagttggtatcagcagaaggcaggccagtcccctgtcttggtcatctatcaaagtggccagcggccctcagggatccctgagcgattctctggctccaactctgggaacacagccactctgagtatcagcgagacccaggcgctggatgaggctgactattattgtctgacgtgggaccgcggcactcctgtcttcggacctgggaccacagtcaccgtcgtag

SEQ ID NO: 19 - DNA, 364 bp, Artificial sequence
  Synthetic antibody
caggtgcagctggtgcagtctgggcctgaagtgaagaagcctggggcctcagtgaaggtctcctgtcagacttctggatataccttcaccgattatgaaatgaactgggtgcgacaggcccctggacagggccttgagtggatgggattaataaacccgcacagtggtgacacaggctatgcactgaagttccagggcagagtcaccatgaccagcgacacctccaaaagcacagtgtacttggagttgagtggcctgacatctgaagacacggccgtatattactgtgcgaggagtcagggaaccttcgaggtttactactttgtctcctggggccagggatccctagtcaccgtctcctcag

SEQ ID NO: 20 - DNA, 325 bp, Artificial sequence
  Synthetic antibody
tcctatgttctgactcagccaccttcggtgtcagtggccccagggaagacggccaccattacgtgtggggctaacggaattggcaggaaacgtgtgcactggtatagccagaggccaggccaggcccctgtggtggtcgtttctgatgatgacgatcgatcctcagtgaaccctgaacgattctctgcctccaaatctggggacacggccgccctgacaatcaccagggtcgaggcgggggatgaggccgattattactgtcaggtgtgggatagtaggactgaccaagtggtgttcggcggagggaccaaggtgaccgtcctgg

SEQ ID NO: 21 - DNA, 364 bp, Artificial sequence
  Synthetic antibody
caggtgcagctggtgcagtctgggcctgaagtgaagaagcctggggcctcagtgaaggtctcctgtaagacttctggatacagcttcaccgattatgaaatgaactgggtgcgacaggcccctggacagggccttgagtggattggattggtaaacccgcacagtggtgagacaggctatgcactgaagttccagggcagagtcaccatgaccagcgacaccgccataagtacagtatacttggagttgagtggcctgacaactgaggacacggccatatattattgtgcgaggagtcagggaaccttcgaggtttattactttgtctcctggggccagggatccctagtcaccgtctcctcag

SEQ ID NO: 22 - skipped

SEQ ID NO: 23 - DNA, 355 bp, Artificial sequence
  Synthetic antibody
caggtgcagctggtggagtcggggggaggcgtggtccagcctgggaggtccctgagactctcctgtgtaggttctggattcacattcagaagggatgctaagtactgggtccgccaggctccaggtaaggggctagaatgggtggcagcgatttcgcatgatggcggtgaggaagactacgcagagtccgtgaagggccgattcaccatttccagagacaattccagggaaacagtttatctggaaatgaacagcctgagacctgaagacacggctgtctattattgtgcgactactcggagtggctggtactttgactactggggtcagggagccctggtcaccgtctcctcag

SEQ ID NO: 24 - DNA, 325 bp, Artificial sequence
  Synthetic antibody
gaaattgtactgacgcagtctccagccaccctgtctttgtctccaggggctagagccaccctctcctgcagggccagtcagagtgttgacacctacttagcctggtaccaacagagacgtggccagtctcccaggctcctcatctatgatgcatccaagagggccactggcatcccagccaggttcagtggcagtgggtctgggacagatttcactctcaccatcagcagcctagagcctgaagattttgcagtttattactgtcagcagcgtaacaactggcggacgtggacgttcggccaagggaccaaggtggaaatcaacc

SEQ ID NO: 25 - DNA, 364 bp, Artificial sequence
  Synthetic antibody
caggtgcagctggtgcagtctgggcctgacgtgaggaagcctggggccacagtgaaggtctcctgtcagacctctggatataggttcaccgattatgaaatgaactgggtgcgacaggcccctggacagggccttgagtggatagggttgataaatccgcacagtggtgacacagcatatgcacagaaattccagggcagagtcaccatgaccagcgacacatccgatagcacagtctatttggaactgagtggcctgacacctgatgacacggccgtgtactactgtgcgaggagccagggtaccttcgaggtttactactttgtctcctggggccagggatccctagtcaccgtctcctcag

SEQ ID NO: 26 - DNA, 325 bp, Artificial sequence
  Synthetic antibody
tcctatgtgttgactcagcctccctcgatgtcagtggccccagggacgacggccagcattccctgtggggcaaacaacattgccaagaaacgtgttcactggtaccgccagaagccaggccaggcccctgtggtggtcgtctctgatgatgaggatcgatcctcagtgatccctgaccgattctctggctccaaatctggggacacggccaccctgacaatcagcagggtcgaggccggggatgagggcgactattattgtcaggtgtgggatagtaagactgaccacgtggttttcggcggagggaccaaggtgaccgtcctgg

SEQ ID NO: 27 - DNA, 355 bp, Artificial sequence
  Synthetic antibody
gaagtgcagctggtggagtctgggggaggcttggtacagcctggcaggtccctgagactctcctgtgcagcctctggattcacctttgatgattataccatgcactgggtccggcaagctccagggaagggcctggagtgggtctcaagtattcgttggaatagtggtaacttagactatgcggactctgtgaagggccgattcaccatctccagagacaacgccaggcactccctgtatctgcaaatgaacagtctgagagctgaggacacggccttatattactgtgtaaaagataacggcctacggactctagacttctggggccagggaaccctggtcaccgtctcctcag

SEQ ID NO: 28 - DNA, 334 bp, Artificial sequence
  Synthetic antibody
gatattgtgatgactcagtctccactctccctgcccgtcacccctggagagccggcctccatctcctgcaggtctagtcagagcctcctccataggaatggatacaactatttggattggtacctgcagaagccagggcagtctccacagctcctgatctctttggcttctaatcgggcctccggggtccctgacaggttcagtggcagtggatcaggcacagattttacactgaaaatcagcagagtggaggctgaggatgttggggtttattactgcatgcaagctctacaaacttggacgttcggccaggggaccaaggtggaactcaaac

SEQ ID NO: 29 - DNA, 364 bp, Artificial sequence
  Synthetic antibody
caggtgcagctggtgcagtctgggcctgaagtgaggaagcctggggcctcagtgagggtctcctgtacgacttctggttacacattcatcgattatgaaatgaactgggtgcgacaggcccctggacagggccttgagtggatgggattaataaacccgcacagtggtgacacaggctatgcacagaagttccaggacagagtcaccatgaccagcgacacctccttaagcacagcctacttggagttgagtggcctgacatctgaggacacggccgtatattactgtgcgaggagtcagggtaccttcgaggtctactactttgtctcctggggccagggatccctagtcaccgtctcctcag

SEQ ID NO: 30 - DNA, 325 bp, Artificial sequence
  Synthetic antibody
tcctatgtgctgactcagccaccctcggtgtcagtggccccaggaaagacggccagcataacctgtgaggcaaaaaacattgtaagaaaacgtgtgcattggtaccgccagaagccaggccaggccccggtggtggtcgtctctgatgatgaggatcgatcctcagtgatccctgagcgagtctctggctccaaatcgggggacacggccaccctgacaatcagcagggtcgaggccggggatgaggccgattattactgtcaggtgtgggatagtacgactgaccaggtggtattcggcggagggaccaaggtgaccgtcctgg

SEQ ID NO: 31 - DNA, 364 bp, Artificial sequence; identical to SEQ ID NO: 19
  Synthetic antibody

SEQ ID NO: 32-33 - skipped (2 entries)

SEQ ID NO: 34 - DNA, 340 bp, Artificial sequence
  Synthetic antibody
gacatcgtcctgacccagtctccagactccctggctgtgtctctgggcgagagggccaccatcaactgcaagtccagccagaatattttagacaactccaacaataagaacttcatagcttggcaccagcataaaccaggacagcctcctaaactgctcatttactggggttcttcccgggaatccggggtccctgaccgattcagtggcagcgggtctgggacagatttcactctcaccatcagcaacctgcaggctgaagatgtggcagtttattactgtctccaatattatagtcttcctcacacttttggtcaggggaccaaggtggagatcaaac

SEQ ID NO: 35 - DNA, 364 bp, Artificial sequence
  Synthetic antibody
caggtgcagctggtggagtctgggggaggcttggtcaagcctggagggtccctcagactctcctgcgtagcctctggattcaccttcagtgacttctacatgagctggatccgccaggctccagggaagggccttgagtgggtgtcctacatgagtgcaactggcggtaatatatactatgcagactctatgaagggccgattaactatctccagggacaacaccaagaactcattgtttctccaaatgaacagcctgagagccgacgacacggccctgtattattgtgcgaggcggaagtttggtgcagggagtgcgatctttgaccactggagccagggaaccctggtcaccgtctcctcag

SEQ ID NO: 36 - DNA, 319 bp, Artificial sequence
  Synthetic antibody
tcctatgaactgactcaaccaccctcagtgtccgtgtccccaggacagacagccagcgtcacctgctctggagacaaattgggtgaaagatatgtgagttggtatcagcagaaggcaggccagtcccctgacttggtcatctatcaaactaaccagcggccctcagggatccctgagcgattctctggctccgactctgggaacacagccactctgactatcagcgggacccagggtctggatgaggcagactattactgtctgacgtgggaccgcggcactcctgtcttcggaactgggaccaaagtcaccgtcctag

SEQ ID NO: 37 - DNA, 361 bp, Artificial sequence
  Synthetic antibody
gaggtgcagttgttggagtcagggggaggcttggtacagccgggggggtccctgagactctcctgtgcagcctctggattcacctttagcaaccatgccatgagctgggtccgccagactccaggggaggggctgcagtgggtctcagctcttacttatagtggtaagaccacatactacgcagactccgtgaagggccggttcaccatctccagagacaattccaagaatttactatttctgcaaatgaacagcctgagagccggggacacggccatatattactgtgcgaaggaggactacgatgaccggggcttctttgacttctggggccaagggacaagggtcaccgtctcctcag

SEQ ID NO: 38 - DNA, 322 bp, Artificial sequence
  Synthetic antibody
gacatccagatgacccagtctccatcctccctgtctgcgtctgtgggagacagagtcaccatcacttgccgggcaagtcagaccattagtacttatttacattggtatcaacaaaaaccaggcaaagcccctaacctcctcatctatgctgcatccactttgcaaagtggggtcccatcaaggttcagtggcagtggatctgggacagatttcagtctcaccatcagtagtctgcgtcctgaagattttgcaatttactactgtcaacagggttacaataacccgtacacttttggccaggggaccaaagtggatatcaaaa

SEQ ID NO: 39 - DNA, 372 bp, Artificial sequence
  Synthetic antibody
caggtgcagctggtggagtctgggggaggcgtggtccagcctgggaggtcgctgcgactctcctgtgcaggctctggattcaggttcagtgactatggcatgcactgggtccgccaggctccaggcaaggggctggagtgggtggctatagttgcatatgatgagagcaagaaatactatgcagactccgtaaagggccgattcaccatctccagagacaattccaagaacacgctgtttctgcaaatgaacagcctgagagctgacgacacggctgtctattactgtgcgaaacggacccaaaaatttgccccctattatttcaacggtttggatgtctggggccaagggaccacggtcaccgtctcctca

SEQ ID NO: 40 - DNA, 337 bp, Artificial sequence
  Synthetic antibody
gatattgtgatgactcagtctccactctccctgcccgtcacccctggagagccggcctccatctcctgtaggtctagccagagcctcctggatcgtaatggatacaactacttggattggtacgtgcagaagccagggcagtctccacagctcctgatgtatttgggttctgatcgggcctccggggtccctgccaggttcagtggcagtggatcaggcacagattttacactgaaaatcagtagagtggaggttgaggatgttggggtttattactgcatgcaagctctacaaattccgatcaccttcggccaagggacacgactggagattaaac

SEQ ID NO: 41 - DNA, 372 bp, Artificial sequence; identical to SEQ ID NO: 39
  Synthetic antibody

SEQ ID NO: 42 - DNA, 337 bp, Artificial sequence; identical to SEQ ID NO: 40
  Synthetic antibody

SEQ ID NO: 43 - DNA, 385 bp, Artificial sequence
  Synthetic antibody
cagctgcagctccaggagtcgggcccaggactagttaagccttcggagaccctgtccctgacctgctccatttctggtgcctccatcaacagtgatgattattattggggctggattcggcaggccccaggggaggcactggagtggattgcgagtatcgatgctagtgggaccacgttctacaatccgtccctcagaagtcgggtcaccatctccgtggacacgtccacgaaccaaatctccctgaggctgaactctgtgaccgccgcagacacggctatatattactgtgcgagacggaagacctattacgattttttgactgattattacaattggtacttcgatgtctggggccgtggcaccctggtcaccgtctcctcag

SEQ ID NO: 44 - DNA, 334 bp, Artificial sequence
  Synthetic antibody
cagtctgccctgactcagcctcgctcagtgtccgggtctcctgggcagtcagtcaccttctcctgcactggaattaatagtgacattcgtgattatgattatgtctcatggtatcaacaacacccaggcgaggcccccaaactcatcatttatgatgtcactaaacgggcctcaggggtcccttctcgcttctctggctccaagtctggcaacacggcctccctgaccatctctgggctccaggctgaagatgaggcggactattactgctgttcgtattcagtcacttactctttcgaggtctttggaactgggacccaggtctccgtcctag

SEQ ID NO: 45 - DNA, 372 bp, Artificial sequence; identical to SEQ ID NO: 39
  Synthetic antibody

SEQ ID NO: 46 - DNA, 337 bp, Artificial sequence; identical to SEQ ID NO: 40
  Synthetic antibody

SEQ ID NO: 47 - DNA, 364 bp, Artificial sequence
  Synthetic antibody
gaggtgcagctggtggagtcggggggaggcctggtcaagcctggggggtccctgagactctcctgtgcagcctctggattcacgttcagtctgtacaccatgaactgggtccgccaggctccagggaaggggctggagtgggtctcatcgattacgtcaaatagtagtcacttgtactatgttgattccgtgaagggccggttcaccatctccagagacaactccaagaactcagtgtctctgcaaatgaacagcctgagagccgaggacacggctgtgtattattgtacgaggtcttacagcagcaaatatgacaactggttcgacccctggggccaggggatgctggtcaccgtctcctcag

SEQ ID NO: 48 - DNA, 322 bp, Artificial sequence
  Synthetic antibody
tcctatgagctgactcagccaccctccgtgtccgtgtccccaggacagacagccagcatcatgtgctctggagataacttgggggataagtatgtttgctggtatcaacagaagccaggccactcccctgaattggtcatgtatcgagataataagaggccctcaggggtccctgagcgattctctggctccaagtctgggaacactgccactctgaccatcagcgggtcccaggccatggatgaggctgactattactgtcaggcgttggacagtggcagtttttgggtgttcggcggagggaccaagctgaccgtcctgg

SEQ ID NO: 49 - DNA, 373 bp, Artificial sequence
  Synthetic antibody
caggtgcagctggtggagtctgggggaggcgtggtccagcctgggaggtcgctgcgactctcctgtgcaggctctggattcaggttcagtgactatggcatgcactgggtccgccaggctccaggcaaggggctggagtgggtggctatagttgcatatgatgagagcaagaaatactatgcagactccgtaaagggccgattcaccatctccagagacaattccaagaacacgctgtttctgcaaatgaacagcctgagagctgacgacacggctgtctattactgtgcgaaacggacccaaaaatttgccccctattatttcaacggtttggatgtctggggctaagggaccacggtcaccgtctcctcag

SEQ ID NO: 50 - DNA, 337 bp, Artificial sequence; identical to SEQ ID NO: 40
  Synthetic antibody

SEQ ID NO: 51 - DNA, 367 bp, Artificial sequence
  Synthetic antibody
cagctgcagctgcaggagtcgggcccaggactggtgaagccttcggagaccctgtcactcacctgcagtgtctctgatgactccatcagtactcctagttacttctggacctggatccgccagcccccagggaaggggctggagtggatagccagtatctattatactgggaccacctactacaacccgtccctcaagagtcgagtcaccttatccgtcgacacgcccaagaggcagttcttcctgaggctgagctctgtgaccgccgcagacacggctgtttattactgtgcgagatatcttgattacgtttggttgagggcttttgatgtctggggccaaggggcaatggtcaccgtctcttcag

SEQ ID NO: 52 - DNA, 322 bp, Artificial sequence
  Synthetic antibody
gaaattgtgttgacacagtctccagccaccctgtctttgtctccaggggaaagagccaccctctcctgcagggccagtccgagtgttggcaggttcttagcctggtaccagcagaaacctggccaggctcccaggctcctcatctatgatgcatctcagagggccactgacatcccagccaggttcagtgccagtgggtctgggacagacttcactctcaccatcgacagcctagagcctgaagattttgcaatatattactgtcagcaccgtagcaactggccggtcactttcggtggagggaccagggtggagatcaagc

SEQ ID NO: 53 - DNA, 373 bp, Artificial sequence
  Synthetic antibody
gaggtgcagctggtggagtctgggggaggcctggtcaagcctggggggtccctgaggctctcctgtataggctctggattcgacttcagtagagatactttccactgggtccgccaggctcccgggaaggggctggagtggatttcaagcatcagtcgtattgagacttacacatactacgtcgactcagtgaagggccgattcaccgtctccagagacaacgccaagaattcagtctatctacaaatgaacagcctgagagtcgaagacacggctgtctatttttgtgcgagagcaggagcagttcgtcccggaattggatttcactactttgacaattggggccagggaagcccggtcaccgtctcctcag

SEQ ID NO: 54-55 - skipped (2 entries)

SEQ ID NO: 56 - DNA, 322 bp, Artificial sequence
  Synthetic antibody
gacatccagatgacccagtctccttccaccgtgtctgcatctgtaggagacagagtcaccatcacttgtcgggccagtcagagcattggtcgctggttggcctggcatcagcagaagccagggaaagccccgaaagtcctgattactagggcctctaatgtagaaagtggggtcccatcaaggttcagcggcagtggatctgggacagaattcactctcaccatcagcagcctgcagcctgatgattttgcaacttattactgtcaacaatataatactaattcggggacatccggccaagggaccaaggcggaaatcaaac

SEQ ID NO: 57 - DNA, 367 bp, Artificial sequence
  Synthetic antibody
caggtgcagctggtggagtctgggggaggcttggtcaagcctggagggtccctgagactctcctgtacagcctctggattcaactttactaactactacatgagctggatccgccaggctccagggaagggcctggagtgggtttcatacattagtagtactactaatagcatagattacgcagactctgtgaagggccgattcaccatctccagggacaacgccaagaagtcactgtatctgcagatgaacagcctaagagccgacgacacggccttctattactgtgcgcgacatttggtcagggggacctctcttgctgcttttgatatctggggccaagggacaatggtcaccgtctcctcag

SEQ ID NO: 58 - skipped

SEQ ID NO: 59 - DNA, 345 bp, Artificial sequence
  Synthetic antibody
caggtgcagctggtgcaatctgggtctgagttgaggaagcctggggcctcagtgaaggtttcctgcaaggcttccggatacaccttcactaagtatggtatgaattgggtgcgacaggcccctggacaaggactggagtggatgggatggattaacacgaacactgcaaagccaacgtatgcccaggacttcacaggacgatttgtcttctctttggacacctctgtcaacacggcatatctggagatcagcggcctaaaggctgaagacaccgccgtctattactgtgcgacagatggtagtgagggctcctggggccagggaaccctggtcaccgtctcctca

SEQ ID NO: 60 - DNA, 322 bp, Artificial sequence
  Synthetic antibody
gacatccagatgacccagtctccttccaccctgtctgcatctgtaggagacagagtcaccatcacttgccgggccagtcagagtattggtacctggttggcctggcatcagcagaaaccagggacagcccctaaggtcctgatctataaggcgtctaatttaaaaagtggggtcccatctagattcagcggcagtggatctgggacagacttcactctcaccatcagcagcctgcagcctgatgatgttgcaacttattactgtcaacaatataatacttactcggggacgttcggccaagggacccgggtggagatcaaac

SEQ ID NO: 61 - DNA, 361 bp, Artificial sequence
  Synthetic antibody
caggtgcagctacagcagtggggcgcaggactgttgaagccttcggagaccctgtccctcacctgcgctgtctatggtgggtccttcaatggttactactggagttggatccgccagcccccagggaaggggctggagtggattggggaaatcgatcatagtggaagcaccaactacaactcgtccctcaagagtcgagtcaccatatcagtagacacgtccaagaaccagttctccctgaagctgagctccgtgaccgccgcggacacggctgtgtattattgcgcgaggaggcgcattgggagcctattaaaatacttccaggactggggccagggcaccctggtcaccgtctcctcag

SEQ ID NO: 62 - DNA, 322 bp, Artificial sequence
  Synthetic antibody
tcctatgagctgactcagccaccctcagtgtccgtgtccccaggacagacagccagcatcacctgctctggagataaattgggggatagttatgtttgttggtatcaactgaagccaggccagtcccctgtgctggtcatctatcaagataccaaacggccctcagggatccctgagcgattttctggctccaactctgggaacacagccactctgaccatcagcgagacccaggctatggatgaggctgactattactgtcaggcgtgggacaccaccacggattgggtgttcggcggagggaccaagctgaccgtcctac

SEQ ID NO: 63 - DNA, 367 bp, Artificial sequence
  Synthetic antibody
cagctgcagctgcaggagtcgggcccaggcctggtgaagccttcggagaccctgtccctcacctgcactgtgtctgatgcctccatcgacactccgagttacttctggagctggatccgccagcccccagggaaggggctggagtggattggcagcatctattatactgggaacaagtactccaatccgtccctcaagagtcgagtcaccatgtccgtagacacgcccaagaggcagttctccctgaggctcagctctgtgaccgccgcagacacggctgtttattactgtgcgagatatgttgattatgtttggttgagggcttttgatatatggggccaagggacaagggtcaccgtctcctcag

SEQ ID NO: 64 - DNA, 322 bp, Artificial sequence
  Synthetic antibody
gaaattgtgttgacacagtctccagccacgctgtctttgtctccaggggaaagggccaccctctcatgcagggccagtccgagtgctggccgcttcttagcttggtaccaacagagacctggccaggctcccaggctcctcatctatgatgcatccaagagggccactgacaccccagccaggttcagtggcagcgggtctgggacagacttcaatcttaccatcgccagcctagagcctgaagattttgcagtttattactgtcaacaccgtagcaactggccgctcactttcggcggagggaccaaggtggagatcaaac

SEQ ID NO: 65 - DNA, 367 bp, Artificial sequence
  Synthetic antibody
gaggtgcagctggtggagtccgggggaggcttagttcggcctggggggtccctgagactctcctgtgtcgcctctggattcaccttcaatacttactggatgcactgggtccgccaagctccagggaaggggctactatgggtctcatctctgaataaagatggcagtagtccaacgtacgcggactccgtgaagggccggttcaccatctccagagacaacaccaacaacacgctctttctccagttggacagtctgagagccgaggacacggctgtctattactgtgtcagagatcgacgagcggtgactacggcccggtacttcgatctctggggccgtggcaccctcgtcaccgtctcctcag

SEQ ID NO: 66 - DNA, 325 bp, Artificial sequence
  Synthetic antibody
gacatcgtgatgacccagtctccagagtccctggctgtgtctctgggcgagagggccaccatcaactgcaagtccagctccaacattgataactacttaggttggtaccagcagagaccaggacagcctcctaaactgctcatttactgggcatctaagcgggaatccggggtccctgaccgattcagtggcagcgggtccgggacagatttcacgctcaccatcggccgcctgcaggctgaagatgtggcagtttattactgtcaacaatattatactagtcctccggtcacttttggcggagggaccaaggtggagatcaaac

SEQ ID NO: 67 - DNA, 346 bp, Artificial sequence
  Synthetic antibody
caggtgcagctggtgcaatctgagtctgagttgaagaagcctggggcctcagtgaagatttcctgcaaggcttctggatacgacctcactaaatatgctatgaattgggtgcggcaggcccctggacaagggcttgagtggatgggatggatcaacaccaacactgcgaaaccaacgtatgcccagggcttcacaggacgatttgtcttctccttggacacgtccgtcagaacgatatatctggagatcagcaacctaaaggctgaggacactgccatttattactgttcgagagatggtagtggggcctcctggggccagggaaccctggtcaccgtctcctcag

SEQ ID NO: 68 - DNA, 322 bp, Artificial sequence
  Synthetic antibody
gacatccagatgacccagtctccttccaccctgtctgcatctgtaggagacagagtcaccatcacttgccgggccagtcagaatattggtcgctggttggcctggcatcagcagaaaccagggaaagcccctaaggtcctgatcactaaggcgtccaatttagagagtggggtcccatccaggttcaccggcagtggctctgggacagacttcactctcaccatcaccagcctgcagcctgatgattttgcgacttattactgtcagcaatatactacttattcggggacgttcggccaagggaccagggtggaaatcaaac

SEQ ID NO: 69 - DNA, 364 bp, Artificial sequence
  Synthetic antibody
gaggtgcagctggtggagtctgggggaggcctggtcaagccgggggggtccctgcgactctcctgtacagtctcgggattcaccttcaatatgcatagcatgaactgggtccgccaggctccagggaaggggctggagtgggtctcattcattagtagtactagcacttacatctactatccagactcagtgaagggccgattcaccgtctccagagacaactccaagaactcactgtatctgcaaatgaccagcctgagagccgaggacacggctgtctattactgtgtgagacgaggtcggggtggagcagcccgtgcacttgacaactggggccagggtaccctggtcaccgtctcctcag

SEQ ID NO: 70 - DNA, 325 bp, Artificial sequence
  Synthetic antibody
tcctatgttctgcctcagacaccctcgctgtcagtggccccaggacagacggccacaatttcctgtgggggaaacaacactggaagtaaaagtgtgcactggtatcaacagaagccaggccaggcccctcttctggtcgtctacgataatagcgaccggccctcagggatccctgagcgattctctgggtccaactctggaaacacggccaccctgaccctcagcagggtcgaagccgaggatgaggccgactattactgtcaggtgtgggatagtactattgatcatgtgatattcggcggcgggaccaagctgaccgtcctag

SEQ ID NO: 71 - DNA, 358 bp, Artificial sequence
  Synthetic antibody
gaggtgcagctggtggagtctgggggaggcctggtcaagccgggggggtccctgagactctcctgtgcagcctctggattcattctcagtaggagtagcatgagctgggtccgccaggctccagggaaggggctggagtgggtctcatatattagtagtactagtagtcatatatattatgcagattcattgaagggccgattcaccatctccagagacaacaccgagaactctgtatatctgcaaatgagtagcctgagagccgaggacacgggtgtctattactgtgcgagaaaaaccaatggggcctggttcctcgatctctggggccgtggcaccctggtcaccgtctcctcag

SEQ ID NO: 72 - DNA, 337 bp, Artificial sequence
  Synthetic antibody
cagtctgtgctgactcagccgccctcagtgtctggggccccaggacagagggtcaccatctcctgcactgggagcagctccaacatcggggcaggttctgatgtgcactggtatcagcaatttccagggtcagccccccacgtcctcatctatggaaataaccaacggccctcaggagtccctgaccggttctctgcctccaagtctggcacctcagcctccctggccatcactgggctgcaggctgacgatgaggctgactattactgccagtcctatgactacgacctgagtgggtcttgggtcttcggcggagggaccaagctgaccgtcctgc

SEQ ID NO: 73 - DNA, 346 bp, Artificial sequence; identical to SEQ ID NO: 67
  Synthetic antibody

SEQ ID NO: 74 - skipped

SEQ ID NO: 75 - DNA, 346 bp, Artificial sequence
  Synthetic antibody
caggtgcagctggtgcaatctgggtctgagttgaggaagcctggggcctcagtgaaggtttcctgcaaggcttccggatacaccttcactaagtatggtatgaattgggtgcgacaggcccctggacaaggactggagtggatgggatggattaacacgaacactgcaaagccaacgtatgcccaggacttcacaggacgatttgtcttctctttggacacctctgtcaacacggcatatctggagatcagcggcctaaaggctgaagacaccgccgtctattactgtgcgacagatggtagtgagggctcctggggccagggaaccctggtcaccgtctcctcag

SEQ ID NO: 76 - DNA, 322 bp, Artificial sequence; identical to SEQ ID NO: 60
  Synthetic antibody

SEQ ID NO: 77 - DNA, 364 bp, Artificial sequence
  Synthetic antibody
caggtgcagctggtgcaatctgggtctgagttgaagaagcctggggcctcaatgaaggtttcctgcaaggcttctggatacaccttcactaactatgctatgaattgggtgcgacaggcccctggacaagggcttgagtggatgggatggatcaacaccaacactgggaaaccaacgtatgcccagggcttcacaggacggtttgtcttctccttggacacctctgtcagtatggcatatctgcagatcagcagcctagaggctggggacactgccgtgtatttctgtgcgagaggcgagtccctagcagccctcggttcgttcgcctactggggccagggaaccctggtcaccgtctcctcag

SEQ ID NO: 78 - DNA, 322 bp, Artificial sequence
  Synthetic antibody
gacatccagatgacccagtctccatcctccctgtctgcatctgtaggagacagagtcaccatcacttgccgggcaagtcagagcattaacagctatttagcttggtatcagcagaaaccagggaaagcccctaacctcctgatctatgctgcatccaatttgcaaagtggggtcccatcacggttcagtggcagtggatctgggacagatttcactctcaccatcagcagtctgcaacctgaagattttgcaacttattactgtcaacagagttccagtactttttggacgttcggccaagggaccaaggtggaaatcaaac

SEQ ID NO: 79 - DNA, 373 bp, Artificial sequence
  Synthetic antibody
gaggtgcagttgttggagtctgggggagacttggtacatcctggggggtccgtgagactctcctgtgccgcctccttttttaactttagaatgtatcccatgagctgggtccgccaggctccagggaaggggctggagtgggtctcgactattagtggaactggtcagaccacatactatgcggactccgtgcagggccgcttcaccatctccagagacaattccaacaacactctttatctacacatgggcagcctgcgagccgacgacacggccaagtattactgtgcgcaactgcatctcggttattatcatgagagcagtggttattttttaagttggggccggggaaccctggtcaccgtctcctcag

SEQ ID NO: 80 - DNA, 319 bp, Artificial sequence
  Synthetic antibody
gacatccagatgacccagtctccttccaccctgtctgcatctgtaggagacacagtcaccatcacttgccgggccagtcagactataagtagttggttggcctggtatcagcagaaaccaggaaaagcccctaacctcctgctttataaggcgtctactttagaaagtggggtcccttcaaggttcagcggcagtggatctgggacagaattcactctcaccatcagaagcctgcagcctgaggattttgcaacttattactgccaacagtatactagttcttggacgttcggccaagggaccaaggtggaaatcaaac

SEQ ID NO: 81 - DNA, 375 bp, Artificial sequence
  Synthetic antibody
cagctgcagctgcaagagtcgggcccaggactggtgaagccttcggagaccctgtccctcacctgcactgtctctggtggctccatcagcagtaggactttcttctggggctggatccgccagcccccagggaagggactagagtggattgggactctcttttccggtgggaccacctaccacaacccgtccctcacgagtcgactcaccatttccgtggacacgtccaggaaccagttctccctgaaactgagttctgtgaccgccgcagacacggctgtgtattactgtgcgagacaacgggcagcattaccgccctactactactactacttcgacgtctggggcaaagggaccacggtcaccgtctcctca

SEQ ID NO: 82 - DNA, 325 bp, Artificial sequence
  Synthetic antibody
gaaattgtgttgacacagtctccagccaccctgtctttgtctccaggggaaagagccaccctctcctgcagggccagtcagagtgttggcagatacttagtctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtggcaactggcctccgatcaccttcggccaagggacacgactggagattaaac

SEQ ID NO: 83 - DNA, 373 bp, Artificial sequence
  Synthetic antibody
gaggtgcagctggtggagtcggggggaggcttggtgcggcctggggaatccctgagactctcctgtgaaggctctggattcaggttcaatgagcatagtctgaattgggtccgccaggctccagggaaggggctggagtggcttgcatacattactgacagtagcggaaataccatacactatgcagactccgtgaagggccgcttcaccatctccagagacaacaccaagaattcagtgtatctgcagatgaacagcctgagacccgaagacacggctgtgtattactgtgcgagagatgggggttgtaatggtcgcacctgctacggactcaactactggggccagggaaccgtggtcaccgtctcctcag

SEQ ID NO: 84 - DNA, 331 bp, Artificial sequence
  Synthetic antibody
cagtctgtgttgacgcagacgccctcaatatctgcggccccaggacagaaggtcaccatctcctgctctggaagcagatccgacattgggaatagttttgtgtcctggtaccagcaattcccaggctcagcccccaaactcctcatttatgacacttttaagcgaccctctgggattcctgaccgcttctctggctccaagtctggcacgtcagccacactggccatcaccgggctccaggctggggacgaggccgtttattactgcggaacatgggatatcagcctgagtgcggcggtcttcggcggagggaccatgctgaccgtcctag

SEQ ID NO: 85 - DNA, 376 bp, Artificial sequence
  Synthetic antibody
caggtgcagctggtggagtctgggggaggcgtggtccagcctggggggtccctgagactctcctgtgcaacgtctggattcaccttcagtagttatggcatgcactgggtccgccaggctccaggcaaggggctggagtgggtggcatttatacggtataatggaagtaataaatactatgcagactccgtgaagggccgattcaccatctccagagacacttccaagaacacgctgtatctgcaaatgaacagcctgagagctgaggactcggctgtgtattactgtgcgaaagacggcgattacgattcttggagtggtctcactgaacacttccagcactggggccagggcaccctggtcaccgtctcctcag

SEQ ID NO: 86 - DNA, 340 bp, Artificial sequence
  Synthetic antibody
gacatcgtgatgacccagtctccagactccctggctgtgtctctgggcgagagggccaccatcaactgcaagtccagccagaatgttttatacagctccaacaataagaactacttagcttggtaccagcagaaaccaagacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtatacctccggctttcggccctgggaccaaagtggaaatcaaac

SEQ ID NO: 87 - DNA, 349 bp, Artificial sequence
  Synthetic antibody
caggtgcagctgatggagtctgggggaggcgtggtccagccgggggggtccctgagactctcctgtgcggcgtctggattcgacttccctggctatggcatgcactgggtccgccagactcccgacaaggggctggagtgggtggcatatatatggtacgatgccagaagtgaagactatgtagactccgtgaagggccgattcaccatttccagagacaactccaacaacacgttgtatctcaagatgaccgatctgagacctcaggacacggctatgtattattgtgcggtgggagctcctctcgaagggtactggggccagggaacccgggtcaccgtctcctcag

SEQ ID NO: 88 - DNA, 325 bp, Artificial sequence
  Synthetic antibody
gacatccagatgacccagtctccatcctccctgtctgcatctgtaggggacaaggtcaccatcacttgccgggcaagtcagaacattgccgactatttaagttggtttcagcaaaaaccagggaaagcccctaaaatcctgatctatgctgcatctactttgcaaagtggggtcccatcaaggttcagtggcagtggatctgggacacatttcactctcaccatcagcagtctgcagcccgaagattttgcaagttacttctgtcagcagagttacacttcacccacgtggacgttcggccaagggaccaaggtggaagtcaaac

SEQ ID NO: 89 - DNA, 367 bp, Artificial sequence
  Synthetic antibody
caggtgcagctgcaggagtcgggcccaggactggtgaagccttcggagaccctgtccctcacctgcactgtctctggtggctccatcagtagttactactggacctggatccggcagcccgccgggaagggactggagtggattgggcgtatctttaccactgggagcaccaattataatccctccctcaagagtcgagtcaccatgtcagtagacacgtccaagaaccagttctccctacacctgacctctgtgaccgccgcggacacggccgtatattattgtgcgagactgaggagggtagtaccaactgctatctggcacttcgatctctggggccgtggcacccgggtcaccgtctcctcag

SEQ ID NO: 90 - DNA, 331 bp, Artificial sequence
  Synthetic antibody
cagactgtggtgacccaggagccatcgatctcagtgtcccctggagggacagtcacactcacttgtggcttgagctctggcgcagtctctactagttacttccccagctggtaccagcagatcccaggccaggcaccacgcacgctcatctacggcacaaacactcgctcttctggggtccctgatcgcttctctggctccatccttgggaacaaagctgccctcaccatcacgggggcccaggcagatgatgaatgtgattattactgtgtgttgtatatggatagtggcgtgatggtattcggcggagggaccaagctgaccgtcctag

SEQ ID NO: 91-128 - skipped (38 entries)

SEQ ID NO: 129 - protein, 121 aa, Artificial sequence
  Synthetic antibody
EVQLVESGGDLVQPGGSLRLSCAASRFSFGSYAMSWVRQAPGKGLEWVSAISSSGGRAYYADSVKGRFTISRDNSRNTLYLQLNSLRGEDTAFYYCTKEGDTGSLWLFDSWGQGTLVTVSS

SEQ ID NO: 130 - protein, 113 aa, Artificial sequence
  Synthetic antibody
DIVMTQSPDSLAVSLGERATINCKSSQSVLDSSNNKKYLAWYQQKPGQPPKLLIYWASTREFGVPDRFSGSGSGTDFTLTISSLQAEDVAVYYCQQYYSTPPTFGQGTKVEIK

SEQ ID NO: 131 - protein, 123 aa, Artificial sequence
  Synthetic antibody
EVQLVESGGALVKPGGSLRLSCEGSGFMFSSAWLHWVRQAPGMGLELVGRMKSETDGGTLDYTAPVRGRFTISRDDSKNMFFLQMNSLKAEDTAVYYCVTGNFDYRSSPLGFWGQGALVTVSS

SEQ ID NO: 132 - protein, 107 aa, Artificial sequence
  Synthetic antibody
DIQMTQSPSTLSASAGDRVTITCRASENIYKWLAWYQQKPGKAPKLLIYKVSTLESGVPSRFSGSGFGTEFTLTISSLQPGDFATYYCQHYNSFPFTFGQGTKLEVK

SEQ ID NO: 133 - protein, 121 aa, Artificial sequence
  Synthetic antibody
EVQLVQSGGGLVQPGGSLRLSCAASRFIFGSYAMSWVRQAPGKGLEWVSAISSSGGRTYYADSVKGRFTISRDNSRNTLYLQLSSLRAEDTAFYYCTKEGDTGSLWLFDSWGQGTLVTVSS

SEQ ID NO: 134 - protein, 113 aa, Artificial sequence
  Synthetic antibody
DIVMTQSPDSLAVSLGERATINCKSSQSVLDSSNNKKYLAWYQQKPGQSPKLLIYWASTREFGVPDRFSGSGSGTDFTLTISSLQAEDVAVYYCQQYYTTPPTFGRGTKVEIK

SEQ ID NO: 135 - protein, 121 aa, Artificial sequence
  Synthetic antibody
EVQLVESGGGLVKPGGSLKLSCAASGFTFSDYYMYWVRQTPEKRLEWVATISDGGSYTYYPDNVKGRFTISRDNAKNTLYLQMSHLKSEDTAMYYCARDKGPIVPMPMDYWGQGTSVTVSS

SEQ ID NO: 136 - protein, 106 aa, Artificial sequence
  Synthetic antibody
SYELTQPLSVSVSPGQTVNITCSGDKLGAKFTCWYQQKPGQSPILVLYQDTKRPPGIPERFSGSNSGNTATLTISGTQAMDEADYYCQAWESGIVAFGGGTKLTVV

SEQ ID NO: 137 - protein, 122 aa, Artificial sequence
  Synthetic antibody
QVQLVESGGRVVQPGESLRLSCETSGFPFHDHGMHWVRQAPGKGLEWVAFIRFDSTSKYTSDSVRGRFSISRDNSKSTLFLQMNNLRREDTAIYYCARDANARFGVMIMAHWGQGTLVTVSS

SEQ ID NO: 138 - protein, 108 aa, Artificial sequence
  Synthetic antibody
DIQLTQSPSSLSASVGDRVTITCQASQDIGHYLNWYLQKPGQAPQVLIYDASNLVTGVPSRFSGSGSGTHFTFTISSLQPEDFGTYYCHQHEKLYSISFGQGTRLDIK

SEQ ID NO: 139 - protein, 121 aa, Artificial sequence
  Synthetic antibody
QVQLVQSGPEVRKPGASVKVSCKTSGYMFTDYEMNWVRQAPGQGLEWMGLINPHSGDTGYAQKFQGRVSMTSDTSTRTFYLELTGLTSEDTAVYYCARSQGTFEVYYFVSWGQGSLVTVSS

SEQ ID NO: 140 - protein, 108 aa, Artificial sequence
  Synthetic antibody
SYVLTQPPSVSVAPGKTASITCGAKNIATKRVHWYRQKPGQAPVVVVSDEEDRSSVIPERFSGSKSGDTATLEISRVEAGDEADYYCQVWDSLADQVVFGGGTKVTVL

SEQ ID NO: 141 - protein, 130 aa, Artificial sequence
  Synthetic antibody
QVQLVQSGSELKKPGASVKVSCEASGYNLTTYAMNWVRQAPGQGLEWMGWINTNTGNPTYAQDFTGHFVFSLDTSVSTAYLQISSLKAEDTAVYYCARDFGERGNCVNGVCYGGYGMDVWGQGTTVTVSS

SEQ ID NO: 142 - protein, 107 aa, Artificial sequence
  Synthetic antibody
DIQMTQSPSTLSASVGDRVTITCRASQSISRWLAWYQQKPGKVPKLLIYKASRLEGGVPSRFSGSGSGTEFTLTISSLQPDDFATYYCQEYTNYSGTFGQGTKVEIR

SEQ ID NO: 143 - protein, 121 aa, Artificial sequence
  Synthetic antibody
QVQLVQSGPEVKKPGASVKVSCKTSGYTFTDSEMNWVRQAPGQGLEWMGLINPHSGDTRYAERFQGRVTMTSDTSINTVYLELSGLTSEDTAIYFCARSQGTFEVYYFLSWGQGSLITVSS

SEQ ID NO: 144 - skipped

SEQ ID NO: 145 - protein, 121 aa, Artificial sequence
  Synthetic antibody
QVQLVESGGGLVKPGGSLRLSCAASGFSFSDFYMSWIRQAPGKGLECISYMSSSGGNIYYADSVKGRFTISRDNAKNSLSLQMNSLRADDTALYYCARRRYGSGSSIFDYWSQGTLVTVSS

SEQ ID NO: 146 - protein, 106 aa, Artificial sequence
  Synthetic antibody
SYELTQPPSVSVSPGQTASVTCSGDTLGDRYVSWYQQKAGQSPVLVIYQSGQRPSGIPERFSGSNSGNTATLSISETQALDEADYYCLTWDRGTPVFGPGTTVTVV

SEQ ID NO: 147 - protein, 121 aa, Artificial sequence
  Synthetic antibody
QVQLVQSGPEVKKPGASVKVSCQTSGYTFTDYEMNWVRQAPGQGLEWMGLINPHSGDTGYALKFQGRVTMTSDTSKSTVYLELSGLTSEDTAVYYCARSQGTFEVYYFVSWGQGSLVTVSS

SEQ ID NO: 148 - protein, 108 aa, Artificial sequence
  Synthetic antibody
SYVLTQPPSVSVAPGKTATITCGANGIGRKRVHWYSQRPGQAPVVVVSDDDDRSSVNPERFSASKSGDTAALTITRVEAGDEADYYCQVWDSRTDQVVFGGGTKVTVL

SEQ ID NO: 149 - protein, 121 aa, Artificial sequence
  Synthetic antibody
QVQLVQSGPEVKKPGASVKVSCKTSGYSFTDYEMNWVRQAPGQGLEWIGLVNPHSGETGYALKFQGRVTMTSDTAISTVYLELSGLTTEDTAIYYCARSQGTFEVYYFVSWGQGSLVTVSS

SEQ ID NO: 150 - skipped

SEQ ID NO: 151 - protein, 118 aa, Artificial sequence
  Synthetic antibody
QVQLVESGGGVVQPGRSLRLSCVGSGFTFRRDAKYWVRQAPGKGLEWVAAISHDGGEEDYAESVKGRFTISRDNSRETVYLEMNSLRPEDTAVYYCATTRSGWYFDYWGQGALVTVSS

SEQ ID NO: 152 - protein, 108 aa, Artificial sequence
  Synthetic antibody
EIVLTQSPATLSLSPGARATLSCRASQSVDTYLAWYQQRRGQSPRLLIYDASKRATGIPARFSGSGSGTDFTLTISSLEPEDFAVYYCQQRNNWRTWTFGQGTKVEIN

SEQ ID NO: 153 - protein, 121 aa, Artificial sequence
  Synthetic antibody
QVQLVQSGPDVRKPGATVKVSCQTSGYRFTDYEMNWVRQAPGQGLEWIGLINPHSGDTAYAQKFQGRVTMTSDTSDSTVYLELSGLTPDDTAVYYCARSQGTFEVYYFVSWGQGSLVTVSS

SEQ ID NO: 154 - protein, 108 aa, Artificial sequence
  Synthetic antibody
SYVLTQPPSMSVAPGTTASIPCGANNIAKKRVHWYRQKPGQAPVVVVSDDEDRSSVIPDRFSGSKSGDTATLTISRVEAGDEGDYYCQVWDSKTDHVVFGGGTKVTVL

SEQ ID NO: 155 - protein, 118 aa, Artificial sequence
  Synthetic antibody
EVQLVESGGGLVQPGRSLRLSCAASGFTFDDYTMHWVRQAPGKGLEWVSSIRWNSGNLDYADSVKGRFTISRDNARHSLYLQMNSLRAEDTALYYCVKDNGLRTLDFWGQGTLVTVSS

SEQ ID NO: 156 - protein, 111 aa, Artificial sequence
  Synthetic antibody
DIVMTQSPLSLPVTPGEPASISCRSSQSLLHRNGYNYLDWYLQKPGQSPQLLISLASNRASGVPDRFSGSGSGTDFTLKISRVEAEDVGVYYCMQALQTWTFGQGTKVELK

SEQ ID NO: 157 - protein, 121 aa, Artificial sequence
  Synthetic antibody
QVQLVQSGPEVRKPGASVRVSCTTSGYTFIDYEMNWVRQAPGQGLEWMGLINPHSGDTGYAQKFQDRVTMTSDTSLSTAYLELSGLTSEDTAVYYCARSQGTFEVYYFVSWGQGSLVTVSS

SEQ ID NO: 158 - protein, 108 aa, Artificial sequence
  Synthetic antibody
SYVLTQPPSVSVAPGKTASITCEAKNIVRKRVHWYRQKPGQAPVVVVSDDEDRSSVIPERVSGSKSGDTATLTISRVEAGDEADYYCQVWDSTTDQVVFGGGTKVTVL

SEQ ID NO: 159 - protein, 121 aa, Artificial sequence; identical to SEQ ID NO: 147
  Synthetic antibody

SEQ ID NO: 160-161 - skipped (2 entries)

SEQ ID NO: 162 - protein, 113 aa, Artificial sequence
  Synthetic antibody
DIVLTQSPDSLAVSLGERATINCKSSQNILDNSNNKNFIAWHQHKPGQPPKLLIYWGSSRESGVPDRFSGSGSGTDFTLTISNLQAEDVAVYYCLQYYSLPHTFGQGTKVEIK

SEQ ID NO: 163 - protein, 121 aa, Artificial sequence
  Synthetic antibody
QVQLVESGGGLVKPGGSLRLSCVASGFTFSDFYMSWIRQAPGKGLEWVSYMSATGGNIYYADSMKGRLTISRDNTKNSLFLQMNSLRADDTALYYCARRKFGAGSAIFDHWSQGTLVTVSS

SEQ ID NO: 164 - protein, 106 aa, Artificial sequence
  Synthetic antibody
SYELTQPPSVSVSPGQTASVTCSGDKLGERYVSWYQQKAGQSPDLVIYQTNQRPSGIPERFSGSDSGNTATLTISGTQGLDEADYYCLTWDRGTPVFGTGTKVTVL

SEQ ID NO: 165 - protein, 120 aa, Artificial sequence
  Synthetic antibody
EVQLLESGGGLVQPGGSLRLSCAASGFTFSNHAMSWVRQTPGEGLQWVSALTYSGKTTYYADSVKGRFTISRDNSKNLLFLQMNSLRAGDTAIYYCAKEDYDDRGFFDFWGQGTRVTVSS

SEQ ID NO: 166 - protein, 107 aa, Artificial sequence
  Synthetic antibody
DIQMTQSPSSLSASVGDRVTITCRASQTISTYLHWYQQKPGKAPNLLIYAASTLQSGVPSRFSGSGSGTDFSLTISSLRPEDFAIYYCQQGYNNPYTFGQGTKVDIK

SEQ ID NO: 167 - protein, 124 aa, Artificial sequence
  Synthetic antibody
QVQLVESGGGVVQPGRSLRLSCAGSGFRFSDYGMHWVRQAPGKGLEWVAIVAYDESKKYYADSVKGRFTISRDNSKNTLFLQMNSLRADDTAVYYCAKRTQKFAPYYFNGLDVWGQGTTVTVSS

SEQ ID NO: 168 - protein, 112 aa, Artificial sequence
  Synthetic antibody
DIVMTQSPLSLPVTPGEPASISCRSSQSLLDRNGYNYLDWYVQKPGQSPQLLMYLGSDRASGVPARFSGSGSGTDFTLKISRVEVEDVGVYYCMQALQIPITFGQGTRLEIK

SEQ ID NO: 169 - protein, 124 aa, Artificial sequence; identical to SEQ ID NO: 167
  Synthetic antibody

SEQ ID NO: 170 - protein, 112 aa, Artificial sequence; identical to SEQ ID NO: 168
  Synthetic antibody

SEQ ID NO: 171 - protein, 128 aa, Artificial sequence
  Synthetic antibody
QLQLQESGPGLVKPSETLSLTCSISGASINSDDYYWGWIRQAPGEALEWIASIDASGTTFYNPSLRSRVTISVDTSTNQISLRLNSVTAADTAIYYCARRKTYYDFLTDYYNWYFDVWGRGTLVTVSS

SEQ ID NO: 172 - protein, 111 aa, Artificial sequence
  Synthetic antibody
QSALTQPRSVSGSPGQSVTFSCTGINSDIRDYDYVSWYQQHPGEAPKLIIYDVTKRASGVPSRFSGSKSGNTASLTISGLQAEDEADYYCCSYSVTYSFEVFGTGTQVSVL

SEQ ID NO: 173 - protein, 124 aa, Artificial sequence; identical to SEQ ID NO: 167
  Synthetic antibody

SEQ ID NO: 174 - protein, 112 aa, Artificial sequence; identical to SEQ ID NO: 168
  Synthetic antibody

SEQ ID NO: 175 - protein, 121 aa, Artificial sequence
  Synthetic antibody
EVQLVESGGGLVKPGGSLRLSCAASGFTFSLYTMNWVRQAPGKGLEWVSSITSNSSHLYYVDSVKGRFTISRDNSKNSVSLQMNSLRAEDTAVYYCTRSYSSKYDNWFDPWGQGMLVTVSS

SEQ ID NO: 176 - protein, 107 aa, Artificial sequence
  Synthetic antibody
SYELTQPPSVSVSPGQTASIMCSGDNLGDKYVCWYQQKPGHSPELVMYRDNKRPSGVPERFSGSKSGNTATLTISGSQAMDEADYYCQALDSGSFWVFGGGTKLTVL

SEQ ID NO: 177 - protein, 124 aa, Artificial sequence
  Synthetic antibody
QVQLVESGGGVVQPGRSLRLSCAGSGFRFSDYGMHWVRQAPGKGLEWVAIVAYDESKKYYADSVKGRFTISRDNSKNTLFLQMNSLRADDTAVYYCAKRTQKFAPYYFNGLDVWGYGTTVTVSS

SEQ ID NO: 178 - protein, 112 aa, Artificial sequence; identical to SEQ ID NO: 168
  Synthetic antibody

SEQ ID NO: 179 - protein, 122 aa, Artificial sequence
  Synthetic antibody
QLQLQESGPGLVKPSETLSLTCSVSDDSISTPSYFWTWIRQPPGKGLEWIASIYYTGTTYYNPSLKSRVTLSVDTPKRQFFLRLSSVTAADTAVYYCARYLDYVWLRAFDVWGQGAMVTVSS

SEQ ID NO: 180 - protein, 107 aa, Artificial sequence
  Synthetic antibody
EIVLTQSPATLSLSPGERATLSCRASPSVGRFLAWYQQKPGQAPRLLIYDASQRATDIPARFSASGSGTDFTLTIDSLEPEDFAIYYCQHRSNWPVTFGGGTRVEIK

SEQ ID NO: 181 - protein, 124 aa, Artificial sequence
  Synthetic antibody
EVQLVESGGGLVKPGGSLRLSCIGSGFDFSRDTFHWVRQAPGKGLEWISSISRIETYTYYVDSVKGRFTVSRDNAKNSVYLQMNSLRVEDTAVYFCARAGAVRPGIGFHYFDNWGQGSPVTVSS

SEQ ID NO: 182-183 - skipped (2 entries)

SEQ ID NO: 184 - protein, 107 aa, Artificial sequence
  Synthetic antibody
DIQMTQSPSTVSASVGDRVTITCRASQSIGRWLAWHQQKPGKAPKVLITRASNVESGVPSRFSGSGSGTEFTLTISSLQPDDFATYYCQQYNTNSGTSGQGTKAEIK

SEQ ID NO: 185 - protein, 122 aa, Artificial sequence
  Synthetic antibody
QVQLVESGGGLVKPGGSLRLSCTASGFNFTNYYMSWIRQAPGKGLEWVSYISSTTNSIDYADSVKGRFTISRDNAKKSLYLQMNSLRADDTAFYYCARHLVRGTSLAAFDIWGQGTMVTVSS

SEQ ID NO: 186 - skipped

SEQ ID NO: 187 - protein, 115 aa, Artificial sequence
  Synthetic antibody
QVQLVQSGSELRKPGASVKVSCKASGYTFTKYGMNWVRQAPGQGLEWMGWINTNTAKPTYAQDFTGRFVFSLDTSVNTAYLEISGLKAEDTAVYYCATDGSEGSWGQGTLVTVSS

SEQ ID NO: 188 - protein, 107 aa, Artificial sequence
  Synthetic antibody
DIQMTQSPSTLSASVGDRVTITCRASQSIGTWLAWHQQKPGTAPKVLIYKASNLKSGVPSRFSGSGSGTDFTLTISSLQPDDVATYYCQQYNTYSGTFGQGTRVEIK

SEQ ID NO: 189 - protein, 120 aa, Artificial sequence
  Synthetic antibody
QVQLQQWGAGLLKPSETLSLTCAVYGGSFNGYYWSWIRQPPGKGLEWIGEIDHSGSTNYNSSLKSRVTISVDTSKNQFSLKLSSVTAADTAVYYCARRRIGSLLKYFQDWGQGTLVTVSS

SEQ ID NO: 190 - protein, 107 aa, Artificial sequence
  Synthetic antibody
SYELTQPPSVSVSPGQTASITCSGDKLGDSYVCWYQLKPGQSPVLVIYQDTKRPSGIPERFSGSNSGNTATLTISETQAMDEADYYCQAWDTTTDWVFGGGTKLTVL

SEQ ID NO: 191 - protein, 122 aa, Artificial sequence
  Synthetic antibody
QLQLQESGPGLVKPSETLSLTCTVSDASIDTPSYFWSWIRQPPGKGLEWIGSIYYTGNKYSNPSLKSRVTMSVDTPKRQFSLRLSSVTAADTAVYYCARYVDYVWLRAFDIWGQGTRVTVSS

SEQ ID NO: 192 - protein, 107 aa, Artificial sequence
  Synthetic antibody
EIVLTQSPATLSLSPGERATLSCRASPSAGRFLAWYQQRPGQAPRLLIYDASKRATDTPARFSGSGSGTDFNLTIASLEPEDFAVYYCQHRSNWPLTFGGGTKVEIK

SEQ ID NO: 193 - protein, 122 aa, Artificial sequence
  Synthetic antibody
EVQLVESGGGLVRPGGSLRLSCVASGFTFNTYWMHWVRQAPGKGLLWVSSLNKDGSSPTYADSVKGRFTISRDNTNNTLFLQLDSLRAEDTAVYYCVRDRRAVTTARYFDLWGRGTLVTVSS

SEQ ID NO: 194 - protein, 108 aa, Artificial sequence
  Synthetic antibody
DIVMTQSPESLAVSLGERATINCKSSSNIDNYLGWYQQRPGQPPKLLIYWASKRESGVPDRFSGSGSGTDFTLTIGRLQAEDVAVYYCQQYYTSPPVTFGGGTKVEIK

SEQ ID NO: 195 - protein, 115 aa, Artificial sequence
  Synthetic antibody
QVQLVQSESELKKPGASVKISCKASGYDLTKYAMNWVRQAPGQGLEWMGWINTNTAKPTYAQGFTGRFVFSLDTSVRTIYLEISNLKAEDTAIYYCSRDGSGASWGQGTLVTVSS

SEQ ID NO: 196 - protein, 107 aa, Artificial sequence
  Synthetic antibody
DIQMTQSPSTLSASVGDRVTITCRASQNIGRWLAWHQQKPGKAPKVLITKASNLESGVPSRFTGSGSGTDFTLTITSLQPDDFATYYCQQYTTYSGTFGQGTRVEIK

SEQ ID NO: 197 - protein, 121 aa, Artificial sequence
  Synthetic antibody
EVQLVESGGGLVKPGGSLRLSCTVSGFTFNMHSMNWVRQAPGKGLEWVSFISSTSTYIYYPDSVKGRFTVSRDNSKNSLYLQMTSLRAEDTAVYYCVRRGRGGAARALDNWGQGTLVTVSS

SEQ ID NO: 198 - protein, 108 aa, Artificial sequence
  Synthetic antibody
SYVLPQTPSLSVAPGQTATISCGGNNTGSKSVHWYQQKPGQAPLLVVYDNSDRPSGIPERFSGSNSGNTATLTLSRVEAEDEADYYCQVWDSTIDHVIFGGGTKLTVL

SEQ ID NO: 199 - protein, 119 aa, Artificial sequence
  Synthetic antibody
EVQLVESGGGLVKPGGSLRLSCAASGFILSRSSMSWVRQAPGKGLEWVSYISSTSSHIYYADSLKGRFTISRDNTENSVYLQMSSLRAEDTGVYYCARKTNGAWFLDLWGRGTLVTVSS

SEQ ID NO: 200 - protein, 112 aa, Artificial sequence
  Synthetic antibody
QSVLTQPPSVSGAPGQRVTISCTGSSSNIGAGSDVHWYQQFPGSAPHVLIYGNNQRPSGVPDRFSASKSGTSASLAITGLQADDEADYYCQSYDYDLSGSWVFGGGTKLTVL

SEQ ID NO: 201 - protein, 115 aa, Artificial sequence; identical to SEQ ID NO: 195
  Synthetic antibody

SEQ ID NO: 202 - skipped

SEQ ID NO: 203 - protein, 115 aa, Artificial sequence; identical to SEQ ID NO: 187
  Synthetic antibody

SEQ ID NO: 204 - protein, 107 aa, Artificial sequence; identical to SEQ ID NO: 188
  Synthetic antibody

SEQ ID NO: 205 - protein, 121 aa, Artificial sequence
  Synthetic antibody
QVQLVQSGSELKKPGASMKVSCKASGYTFTNYAMNWVRQAPGQGLEWMGWINTNTGKPTYAQGFTGRFVFSLDTSVSMAYLQISSLEAGDTAVYFCARGESLAALGSFAYWGQGTLVTVSS

SEQ ID NO: 206 - protein, 107 aa, Artificial sequence
  Synthetic antibody
DIQMTQSPSSLSASVGDRVTITCRASQSINSYLAWYQQKPGKAPNLLIYAASNLQSGVPSRFSGSGSGTDFTLTISSLQPEDFATYYCQQSSSTFWTFGQGTKVEIK

SEQ ID NO: 207 - protein, 124 aa, Artificial sequence
  Synthetic antibody
EVQLLESGGDLVHPGGSVRLSCAASFFNFRMYPMSWVRQAPGKGLEWVSTISGTGQTTYYADSVQGRFTISRDNSNNTLYLHMGSLRADDTAKYYCAQLHLGYYHESSGYFLSWGRGTLVTVSS

SEQ ID NO: 208 - protein, 106 aa, Artificial sequence
  Synthetic antibody
DIQMTQSPSTLSASVGDTVTITCRASQTISSWLAWYQQKPGKAPNLLLYKASTLESGVPSRFSGSGSGTEFTLTIRSLQPEDFATYYCQQYTSSWTFGQGTKVEIK

SEQ ID NO: 209 - protein, 125 aa, Artificial sequence
  Synthetic antibody
QLQLQESGPGLVKPSETLSLTCTVSGGSISSRTFFWGWIRQPPGKGLEWIGTLFSGGTTYHNPSLTSRLTISVDTSRNQFSLKLSSVTAADTAVYYCARQRAALPPYYYYYFDVWGKGTTVTVSS

SEQ ID NO: 210 - protein, 108 aa, Artificial sequence
  Synthetic antibody
EIVLTQSPATLSLSPGERATLSCRASQSVGRYLVWYQQKPGQAPRLLIYDASNRATGIPARFSGSGSGTDFTLTISSLEPEDFAVYYCQQRGNWPPITFGQGTRLEIK

SEQ ID NO: 211 - protein, 123 aa, Artificial sequence
  Synthetic antibody
EVQLVESGGGLVRPGESLRLSCEGSGFRFNEHSLNWVRQAPGKGLEWLAYITSSGNTIHYADSVKGRFTISRDNTKNSVYLQMNSLRPEDTAVYYCARDGGCNGRTCYGLNYWGQGTVVTVSS

SEQ ID NO: 212 - protein, 110 aa, Artificial sequence
  Synthetic antibody
QSVLTQTPSISAAPGQKVTISCSGSRSDIGNSFVSWYQQFPGSAPKLLIYDTFKRPSGIPDRFSGSKSGTSATLAITGLQAGDEAVYYCGTWDISLSAAVFGGGTMLTVL

SEQ ID NO: 213 - protein, 125 aa, Artificial sequence
  Synthetic antibody
QVQLVESGGGVVQPGGSLRLSCATSGFTFSSYGMHWVRQAPGKGLEWVAFIRYNGSNKYYADSVKGRFTISRDTSKNTLYLQMNSLRAEDSAVYYCAKDGDYDSWSGLTEHFQHWGQGTLVTVSS

SEQ ID NO: 214 - protein, 113 aa, Artificial sequence
  Synthetic antibody
DIVMTQSPDSLAVSLGERATINCKSSQNVLYSSNNKNYLAWYQQKPRQPPKLLIYWASTRESGVPDRFSGSGSGTDFTLTISSLQAEDVAVYYCQQYYSIPPAFGPGTKVEIK

SEQ ID NO: 215 - protein, 116 aa, Artificial sequence
  Synthetic antibody
QVQLMESGGGVVQPGGSLRLSCAASGFDFPGYGMHWVRQTPDKGLEWVAYIWYDARSEDYVDSVKGRFTISRDNSNNTLYLKMTDLRPQDTAMYYCAVGAPLEGYWGQGTRVTVSS

SEQ ID NO: 216 - protein, 108 aa, Artificial sequence
  Synthetic antibody
DIQMTQSPSSLSASVGDKVTITCRASQNIADYLSWFQQKPGKAPKILIYAASTLQSGVPSRFSGSGSGTHFTLTISSLQPEDFASYFCQQSYTSPTWTFGQGTKVEVK

SEQ ID NO: 217 - protein, 122 aa, Artificial sequence
  Synthetic antibody
QVQLQESGPGLVKPSETLSLTCTVSGGSISSYYWTWIRQPAGKGLEWIGRIFTTGSTNYNPSLKSRVTMSVDTSKNQFSLHLTSVTAADTAVYYCARLRRVVPTAIWHFDLWGRGTRVTVSS

SEQ ID NO: 218 - protein, 110 aa, Artificial sequence
  Synthetic antibody
QTVVTQEPSISVSPGGTVTLTCGLSSGAVSTSYFPSWYQQIPGQAPRTLIYGTNTRSSGVPDRFSGSILGNKAALTITGAQADDECDYYCVLYMDSGVMVFGGGTKLTVL

SEQ ID NO: 219 - DNA, 21 bp, Artificial sequence
  Synthetic antibody
  modified_base (1)..(20): phosphorothioate-modified oligodeoxynucleotide
tcgtcgtttttcggtcgtttt

SEQ ID NO: 220 - protein, 16 aa, Artificial sequence
  Synthetic antibody
CTKEGDTGSLWLFDSW

SEQ ID NO: 221 - protein, 16 aa, Artificial sequence
  Synthetic antibody
CVTGNFDYRSSPLGFW

SEQ ID NO: 222 - protein, 15 aa, Artificial sequence
  Synthetic antibody
CAKEDYDDRGFFDFW

SEQ ID NO: 223 - protein, 16 aa, Artificial sequence
  Synthetic antibody
CARSQGTFEVYYFVSW

SEQ ID NO: 224 - protein, 16 aa, Artificial sequence
  Synthetic antibody
CARRKFGAGSAIFDHW

SEQ ID NO: 225 - protein, 11 aa, Artificial sequence
  Synthetic antibody
CAVGAPLEGYW

SEQ ID NO: 226 - protein, 13 aa, Artificial sequence
  Synthetic antibody
CVKDNGLRTLDFW

SEQ ID NO: 227 - protein, 17 aa, Artificial sequence
  Synthetic antibody
CARDANARFGVMIMAHW

SEQ ID NO: 228 - protein, 25 aa, Artificial sequence
  Synthetic antibody
CARDFGERGNCVNGVCYGGYGMDVW

SEQ ID NO: 229 - protein, 13 aa, Artificial sequence
  Synthetic antibody
CATTRSGWYFDYW

SEQ ID NO: 230 - protein, 16 aa, Artificial sequence
  Synthetic antibody
CARDKGPIVPMPMDYW

SEQ ID NO: 231 - protein, 18 aa, Artificial sequence
  Synthetic antibody
CARLRRVVPTAIWHFDLW

SEQ ID NO: 232 - protein, 20 aa, Artificial sequence
  Synthetic antibody
CAKDGDYDSWSGLTEHFQHW

SEQ ID NO: 233 - protein, 10 aa, Artificial sequence
  Synthetic antibody
CARGTDLNYW

SEQ ID NO: 234 - protein, 16 aa, Artificial sequence
  Synthetic antibody
CARRRIGSLLKYFQDW

SEQ ID NO: 235 - protein, 19 aa, Artificial sequence
  Synthetic antibody
CAKRTQKFAPYYFNGLDVW

SEQ ID NO: 236 - protein, 22 aa, Artificial sequence
  Synthetic antibody
CARRKTYYDFLTDYYNWYFDVW

SEQ ID NO: 237 - protein, 16 aa, Artificial sequence
  Synthetic antibody
CTRSYSSKYDNWFDPW

SEQ ID NO: 238 - protein, 10 aa, Artificial sequence
  Synthetic antibody
CATDGSEGSW

SEQ ID NO: 239 - protein, 16 aa, Artificial sequence
  Synthetic antibody
CARYVDYVWLRAFDIW

SEQ ID NO: 240 - protein, 17 aa, Artificial sequence
  Synthetic antibody
CVRDRRAVTTARYFDLW

SEQ ID NO: 241 - protein, 19 aa, Artificial sequence
  Synthetic antibody
CARAGAVRPGIGFHYFDNW

SEQ ID NO: 242 - protein, 14 aa, Artificial sequence
  Synthetic antibody
CARKTNGAWFLDLW

SEQ ID NO: 243 - protein, 10 aa, Artificial sequence
  Synthetic antibody
CSRDGSGASW

SEQ ID NO: 244 - protein, 16 aa, Artificial sequence
  Synthetic antibody
CVRRGRGGAARALDNW

SEQ ID NO: 245 - protein, 16 aa, Artificial sequence
  Synthetic antibody
CARGESLAALGSFAYW

SEQ ID NO: 246 - protein, 17 aa, Artificial sequence
  Synthetic antibody
CARHLVRGTSLAAFDIW

SEQ ID NO: 247 - protein, 19 aa, Artificial sequence
  Synthetic antibody
CARQRAALPPYYYYYFDVW

SEQ ID NO: 248 - DNA, 24 bp, Artificial sequence
  Synthetic antibody
agattcagctttggcagctatgcc

SEQ ID NO: 249 - DNA, 24 bp, Artificial sequence
  Synthetic antibody
atttcttctagtggtggtagggca

SEQ ID NO: 250 - DNA, 42 bp, Artificial sequence
  Synthetic antibody
acgaaagaaggtgacacagggtccctatggttatttgactcc

SEQ ID NO: 251 - protein, 8 aa, Artificial sequence
  Synthetic antibody
RFSFGSYA

SEQ ID NO: 252 - protein, 8 aa, Artificial sequence
  Synthetic antibody
ISSSGGRA

SEQ ID NO: 253 - protein, 14 aa, Artificial sequence
  Synthetic antibody
TKEGDTGSLWLFDS

SEQ ID NO: 254 - DNA, 24 bp, Artificial sequence
  Synthetic antibody
ggtttcatgttcagtagcgcctgg

SEQ ID NO: 255 - DNA, 30 bp, Artificial sequence
  Synthetic antibody
atgaaaagtgagactgatggtgggacactt

SEQ ID NO: 256 - DNA, 42 bp, Artificial sequence
  Synthetic antibody
gtcacaggaaacttcgattataggagttcaccccttggcttc

SEQ ID NO: 257 - protein, 8 aa, Artificial sequence
  Synthetic antibody
GFMFSSAW

SEQ ID NO: 258 - protein, 10 aa, Artificial sequence
  Synthetic antibody
MKSETDGGTL

SEQ ID NO: 259 - protein, 14 aa, Artificial sequence
  Synthetic antibody
VTGNFDYRSSPLGF

SEQ ID NO: 260 - DNA, 24 bp, Artificial sequence
  Synthetic antibody
agattcatctttggcagctatgcc

SEQ ID NO: 261 - DNA, 24 bp, Artificial sequence
  Synthetic antibody
attagttctagtggtggcaggaca

SEQ ID NO: 262 - DNA, 42 bp, Artificial sequence
  Synthetic antibody
acgaaagaaggtgacacagggtccctatggctttttgactcc

SEQ ID NO: 263 - protein, 8 aa, Artificial sequence
  Synthetic antibody
RFIFGSYA

SEQ ID NO: 264 - protein, 8 aa, Artificial sequence
  Synthetic antibody
ISSSGGRT

SEQ ID NO: 265 - protein, 14 aa, Artificial sequence
  Synthetic antibody
TKEGDTGSLWLFDS

SEQ ID NO: 266 - DNA, 24 bp, Artificial sequence
  Synthetic antibody
ggattcactttcagtgactattac

SEQ ID NO: 267 - DNA, 24 bp, Artificial sequence
  Synthetic antibody
attagtgatggtggtagttacacc

SEQ ID NO: 268 - DNA, 42 bp, Artificial sequence
  Synthetic antibody
gcaagagataaagggcctatagtccctatgcctatggactac

SEQ ID NO: 269 - protein, 8 aa, Artificial sequence
  Synthetic antibody
GFTFSDYY

SEQ ID NO: 270 - protein, 8 aa, Artificial sequence
  Synthetic antibody
ISDGGSYT

SEQ ID NO: 271 - protein, 14 aa, Artificial sequence
  Synthetic antibody
ARDKGPIVPMPMDY

SEQ ID NO: 272 - DNA, 24 bp, Artificial sequence
  Synthetic antibody
ggattccccttccatgatcacggc

SEQ ID NO: 273 - DNA, 24 bp, Artificial sequence
  Synthetic antibody
attcgatttgattccacttccaag

SEQ ID NO: 274 - DNA, 45 bp, Artificial sequence
  Synthetic antibody
gcgagagatgccaacgcgagatttggagtaatgatcatggcacat

SEQ ID NO: 275 - protein, 8 aa, Artificial sequence
  Synthetic antibody
GFPFHDHG

SEQ ID NO: 276 - protein, 8 aa, Artificial sequence
  Synthetic antibody
IRFDSTSK

SEQ ID NO: 277 - protein, 15 aa, Artificial sequence
  Synthetic antibody
ARDANARFGVMIMAH

SEQ ID NO: 278 - DNA, 24 bp, Artificial sequence
  Synthetic antibody
ggatacatgttcaccgattatgaa

SEQ ID NO: 279 - DNA, 24 bp, Artificial sequence
  Synthetic antibody
ataaacccgcacagtggtgacaca

SEQ ID NO: 280 - DNA, 42 bp, Artificial sequence
  Synthetic antibody
gcgaggagccagggtaccttcgaggtttactactttgtctcc

SEQ ID NO: 281 - protein, 8 aa, Artificial sequence
  Synthetic antibody
GYMFTDYE

SEQ ID NO: 282 - protein, 8 aa, Artificial sequence
  Synthetic antibody
INPHSGDT

SEQ ID NO: 283 - protein, 14 aa, Artificial sequence
  Synthetic antibody
ARSQGTFEVYYFVS

SEQ ID NO: 284 - DNA, 24 bp, Artificial sequence
  Synthetic antibody
ggatacaacctcactacctatgct

SEQ ID NO: 285 - DNA, 24 bp, Artificial sequence
  Synthetic antibody
atcaacaccaacactgggaaccca

SEQ ID NO: 286 - DNA, 69 bp, Artificial sequence
  Synthetic antibody
gcgagagatttcggggagcggggaaattgtgttaatggtgtatgctatgggggttacggtatggacgtc

SEQ ID NO: 287 - protein, 8 aa, Artificial sequence
  Synthetic antibody
GYNLTTYA

SEQ ID NO: 288 - protein, 8 aa, Artificial sequence
  Synthetic antibody
INTNTGNP

SEQ ID NO: 289 - protein, 23 aa, Artificial sequence
  Synthetic antibody
ARDFGERGNCVNGVCYGGYGMDV

SEQ ID NO: 290 - DNA, 24 bp, Artificial sequence
  Synthetic antibody
ggatacacgttcaccgactctgaa

SEQ ID NO: 291 - DNA, 24 bp, Artificial sequence
  Synthetic antibody
ataaatccgcacagtggtgacaca

SEQ ID NO: 292 - DNA, 42 bp, Artificial sequence
  Synthetic antibody
gcgaggagtcaggggaccttcgaggtttactactttctctcc

SEQ ID NO: 293 - protein, 8 aa, Artificial sequence
  Synthetic antibody
GYTFTDSE

SEQ ID NO: 294 - protein, 8 aa, Artificial sequence
  Synthetic antibody
INPHSGDT

SEQ ID NO: 295 - protein, 14 aa, Artificial sequence
  Synthetic antibody
ARSQGTFEVYYFLS

SEQ ID NO: 296 - DNA, 24 bp, Artificial sequence
  Synthetic antibody
ggattcagcttcagtgacttctac

SEQ ID NO: 297 - DNA, 24 bp, Artificial sequence
  Synthetic antibody
atgagtagtagtggtggtaacata

SEQ ID NO: 298 - DNA, 42 bp, Artificial sequence
  Synthetic antibody
gcgagacggaggtatggttcaggaagttcgatctttgactac

SEQ ID NO: 299 - protein, 8 aa, Artificial sequence
  Synthetic antibody
GFSFSDFY

SEQ ID NO: 300 - protein, 8 aa, Artificial sequence
  Synthetic antibody
MSSSGGNI

SEQ ID NO: 301 - protein, 14 aa, Artificial sequence
  Synthetic antibody
ARRRYGSGSSIFDY

SEQ ID NO: 302 - DNA, 24 bp, Artificial sequence
  Synthetic antibody
ggatataccttcaccgattatgaa

SEQ ID NO: 303 - DNA, 24 bp, Artificial sequence
  Synthetic antibody
ataaacccgcacagtggtgacaca

SEQ ID NO: 304 - DNA, 42 bp, Artificial sequence
  Synthetic antibody
gcgaggagtcagggaaccttcgaggtttactactttgtctcc

SEQ ID NO: 305 - protein, 8 aa, Artificial sequence
  Synthetic antibody
GYTFTDYE

SEQ ID NO: 306 - protein, 8 aa, Artificial sequence
  Synthetic antibody
INPHSGDT

SEQ ID NO: 307 - protein, 14 aa, Artificial sequence
  Synthetic antibody
ARSQGTFEVYYFVS

SEQ ID NO: 308 - DNA, 24 bp, Artificial sequence
  Synthetic antibody
ggatacagcttcaccgattatgaa

SEQ ID NO: 309 - DNA, 24 bp, Artificial sequence
  Synthetic antibody
gtaaacccgcacagtggtgagaca

SEQ ID NO: 310 - DNA, 42 bp, Artificial sequence
  Synthetic antibody
gcgaggagtcagggaaccttcgaggtttattactttgtctcc

SEQ ID NO: 311 - protein, 8 aa, Artificial sequence
  Synthetic antibody
GYSFTDYE

SEQ ID NO: 312 - protein, 8 aa, Artificial sequence
  Synthetic antibody
VNPHSGET

SEQ ID NO: 313 - protein, 14 aa, Artificial sequence
  Synthetic antibody
ARSQGTFEVYYFVS

SEQ ID NO: 314 - DNA, 24 bp, Artificial sequence
  Synthetic antibody
ggattcacattcagaagggatgct

SEQ ID NO: 315 - DNA, 24 bp, Artificial sequence
  Synthetic antibody
atttcgcatgatggcggtgaggaa

SEQ ID NO: 316 - DNA, 33 bp, Artificial sequence
  Synthetic antibody
gcgactactcggagtggctggtactttgactac

SEQ ID NO: 317 - protein, 8 aa, Artificial sequence
  Synthetic antibody
GFTFRRDA

SEQ ID NO: 318 - protein, 8 aa, Artificial sequence
  Synthetic antibody
ISHDGGEE

SEQ ID NO: 319 - protein, 11 aa, Artificial sequence
  Synthetic antibody
ATTRSGWYFDY

SEQ ID NO: 320 - DNA, 24 bp, Artificial sequence
  Synthetic antibody
ggatataggttcaccgattatgaa

SEQ ID NO: 321 - DNA, 24 bp, Artificial sequence
  Synthetic antibody
ataaatccgcacagtggtgacaca

SEQ ID NO: 322 - DNA, 42 bp, Artificial sequence; identical to SEQ ID NO: 280
  Synthetic antibody

SEQ ID NO: 323 - protein, 8 aa, Artificial sequence
  Synthetic antibody
GYRFTDYE

SEQ ID NO: 324 - protein, 8 aa, Artificial sequence
  Synthetic antibody
INPHSGDT

SEQ ID NO: 325 - protein, 14 aa, Artificial sequence
  Synthetic antibody
ARSQGTFEVYYFVS

SEQ ID NO: 326 - DNA, 24 bp, Artificial sequence
  Synthetic antibody
ggattcacctttgatgattatacc

SEQ ID NO: 327 - DNA, 24 bp, Artificial sequence
  Synthetic antibody
attcgttggaatagtggtaactta

SEQ ID NO: 328 - DNA, 33 bp, Artificial sequence
  Synthetic antibody
gtaaaagataacggcctacggactctagacttc

SEQ ID NO: 329 - protein, 8 aa, Artificial sequence
  Synthetic antibody
GFTFDDYT

SEQ ID NO: 330 - protein, 8 aa, Artificial sequence
  Synthetic antibody
IRWNSGNL

SEQ ID NO: 331 - protein, 11 aa, Artificial sequence
  Synthetic antibody
VKDNGLRTLDF

SEQ ID NO: 332 - DNA, 24 bp, Artificial sequence
  Synthetic antibody
ggttacacattcatcgattatgaa

SEQ ID NO: 333 - DNA, 24 bp, Artificial sequence
  Synthetic antibody
ataaacccgcacagtggtgacaca

SEQ ID NO: 334 - DNA, 42 bp, Artificial sequence
  Synthetic antibody
gcgaggagtcagggtaccttcgaggtctactactttgtctcc

SEQ ID NO: 335 - protein, 8 aa, Artificial sequence
  Synthetic antibody
GYTFIDYE

SEQ ID NO: 336 - protein, 8 aa, Artificial sequence
  Synthetic antibody
INPHSGDT

SEQ ID NO: 337 - protein, 14 aa, Artificial sequence
  Synthetic antibody
ARSQGTFEVYYFVS

SEQ ID NO: 338 - DNA, 24 bp, Artificial sequence
  Synthetic antibody
ggatataccttcaccgattatgaa

SEQ ID NO: 339 - DNA, 24 bp, Artificial sequence
  Synthetic antibody
ataaacccgcacagtggtgacaca

SEQ ID NO: 340 - DNA, 42 bp, Artificial sequence; identical to SEQ ID NO: 304
  Synthetic antibody

SEQ ID NO: 341 - protein, 8 aa, Artificial sequence
  Synthetic antibody
GYTFTDYE

SEQ ID NO: 342 - protein, 8 aa, Artificial sequence
  Synthetic antibody
INPHSGDT

SEQ ID NO: 343 - protein, 14 aa, Artificial sequence
  Synthetic antibody
ARSQGTFEVYYFVS

SEQ ID NO: 344 - DNA, 24 bp, Artificial sequence
  Synthetic antibody
ggattcaccttcagtgacttctac

SEQ ID NO: 345 - DNA, 24 bp, Artificial sequence
  Synthetic antibody
atgagtgcaactggcggtaatata

SEQ ID NO: 346 - DNA, 42 bp, Artificial sequence
  Synthetic antibody
gcgaggcggaagtttggtgcagggagtgcgatctttgaccac

SEQ ID NO: 347 - protein, 8 aa, Artificial sequence
  Synthetic antibody
GFTFSDFY

SEQ ID NO: 348 - protein, 8 aa, Artificial sequence
  Synthetic antibody
MSATGGNI

SEQ ID NO: 349 - protein, 14 aa, Artificial sequence
  Synthetic antibody
ARRKFGAGSAIFDH

SEQ ID NO: 350 - DNA, 24 bp, Artificial sequence
  Synthetic antibody
ggattcacctttagcaaccatgcc

SEQ ID NO: 351 - DNA, 24 bp, Artificial sequence
  Synthetic antibody
cttacttatagtggtaagaccaca

SEQ ID NO: 352 - DNA, 39 bp, Artificial sequence
  Synthetic antibody
gcgaaggaggactacgatgaccggggcttctttgacttc

SEQ ID NO: 353 - protein, 8 aa, Artificial sequence
  Synthetic antibody
GFTFSNHA

SEQ ID NO: 354 - protein, 8 aa, Artificial sequence
  Synthetic antibody
LTYSGKTT

SEQ ID NO: 355 - protein, 13 aa, Artificial sequence
  Synthetic antibody
AKEDYDDRGFFDF

SEQ ID NO: 356 - DNA, 24 bp, Artificial sequence
  Synthetic antibody
ggattcaggttcagtgactatggc

SEQ ID NO: 357 - DNA, 24 bp, Artificial sequence
  Synthetic antibody
gttgcatatgatgagagcaagaaa

SEQ ID NO: 358 - DNA, 51 bp, Artificial sequence
  Synthetic antibody
gcgaaacggacccaaaaatttgccccctattatttcaacggtttggatgtc

SEQ ID NO: 359 - protein, 8 aa, Artificial sequence
  Synthetic antibody
GFRFSDYG

SEQ ID NO: 360 - protein, 8 aa, Artificial sequence
  Synthetic antibody
VAYDESKK

SEQ ID NO: 361 - protein, 17 aa, Artificial sequence
  Synthetic antibody
AKRTQKFAPYYFNGLDV

SEQ ID NO: 362 - DNA, 24 bp, Artificial sequence
  Synthetic antibody
ggattcaggttcagtgactatggc

SEQ ID NO: 363 - DNA, 24 bp, Artificial sequence
  Synthetic antibody
gttgcatatgatgagagcaagaaa

SEQ ID NO: 364 - DNA, 51 bp, Artificial sequence; identical to SEQ ID NO: 358
  Synthetic antibody

SEQ ID NO: 365 - protein, 8 aa, Artificial sequence
  Synthetic antibody
GFRFSDYG

SEQ ID NO: 366 - protein, 8 aa, Artificial sequence
  Synthetic antibody
VAYDESKK

SEQ ID NO: 367 - protein, 17 aa, Artificial sequence
  Synthetic antibody
AKRTQKFAPYYFNGLDV

SEQ ID NO: 368 - DNA, 30 bp, Artificial sequence
  Synthetic antibody
ggtgcctccatcaacagtgatgattattat

SEQ ID NO: 369 - DNA, 21 bp, Artificial sequence
  Synthetic antibody
atcgatgctagtgggaccacg

SEQ ID NO: 370 - DNA, 60 bp, Artificial sequence
  Synthetic antibody
gcgagacggaagacctattacgattttttgactgattattacaattggtacttcgatgtc

SEQ ID NO: 371 - protein, 10 aa, Artificial sequence
  Synthetic antibody
GASINSDDYY

SEQ ID NO: 372 - protein, 7 aa, Artificial sequence
  Synthetic antibody
IDASGTT

SEQ ID NO: 373 - protein, 20 aa, Artificial sequence
  Synthetic antibody
ARRKTYYDFLTDYYNWYFDV

SEQ ID NO: 374 - DNA, 24 bp, Artificial sequence
  Synthetic antibody
ggattcaggttcagtgactatggc

SEQ ID NO: 375 - DNA, 24 bp, Artificial sequence
  Synthetic antibody
gttgcatatgatgagagcaagaaa

SEQ ID NO: 376 - DNA, 51 bp, Artificial sequence; identical to SEQ ID NO: 358
  Synthetic antibody

SEQ ID NO: 377 - protein, 8 aa, Artificial sequence
  Synthetic antibody
GFRFSDYG

SEQ ID NO: 378 - protein, 8 aa, Artificial sequence
  Synthetic antibody
VAYDESKK

SEQ ID NO: 379 - protein, 17 aa, Artificial sequence
  Synthetic antibody
AKRTQKFAPYYFNGLDV

SEQ ID NO: 380 - DNA, 24 bp, Artificial sequence
  Synthetic antibody
ggattcacgttcagtctgtacacc

SEQ ID NO: 381 - DNA, 24 bp, Artificial sequence
  Synthetic antibody
attacgtcaaatagtagtcacttg

SEQ ID NO: 382 - DNA, 42 bp, Artificial sequence
  Synthetic antibody
acgaggtcttacagcagcaaatatgacaactggttcgacccc

SEQ ID NO: 383 - protein, 8 aa, Artificial sequence
  Synthetic antibody
GFTFSLYT

SEQ ID NO: 384 - protein, 8 aa, Artificial sequence
  Synthetic antibody
ITSNSSHL

SEQ ID NO: 385 - protein, 14 aa, Artificial sequence
  Synthetic antibody
TRSYSSKYDNWFDP

SEQ ID NO: 386 - DNA, 24 bp, Artificial sequence
  Synthetic antibody
ggattcaggttcagtgactatggc

SEQ ID NO: 387 - DNA, 24 bp, Artificial sequence
  Synthetic antibody
gttgcatatgatgagagcaagaaa

SEQ ID NO: 388 - DNA, 51 bp, Artificial sequence; identical to SEQ ID NO: 358
  Synthetic antibody

SEQ ID NO: 389 - protein, 8 aa, Artificial sequence
  Synthetic antibody
GFRFSDYG

SEQ ID NO: 390 - protein, 8 aa, Artificial sequence
  Synthetic antibody
VAYDESKK

SEQ ID NO: 391 - protein, 17 aa, Artificial sequence
  Synthetic antibody
AKRTQKFAPYYFNGLDV

SEQ ID NO: 392 - DNA, 30 bp, Artificial sequence
  Synthetic antibody
gatgactccatcagtactcctagttacttc

SEQ ID NO: 393 - DNA, 21 bp, Artificial sequence
  Synthetic antibody
atctattatactgggaccacc

SEQ ID NO: 394 - DNA, 42 bp, Artificial sequence
  Synthetic antibody
gcgagatatcttgattacgtttggttgagggcttttgatgtc

SEQ ID NO: 395 - protein, 10 aa, Artificial sequence
  Synthetic antibody
DDSISTPSYF

SEQ ID NO: 396 - protein, 7 aa, Artificial sequence
  Synthetic antibody
IYYTGTT

SEQ ID NO: 397 - protein, 14 aa, Artificial sequence
  Synthetic antibody
ARYLDYVWLRAFDV

SEQ ID NO: 398 - DNA, 24 bp, Artificial sequence
  Synthetic antibody
ggattcgacttcagtagagatact

SEQ ID NO: 399 - DNA, 24 bp, Artificial sequence
  Synthetic antibody
atcagtcgtattgagacttacaca

SEQ ID NO: 400 - DNA, 51 bp, Artificial sequence
  Synthetic antibody
gcgagagcaggagcagttcgtcccggaattggatttcactactttgacaat

SEQ ID NO: 401 - protein, 8 aa, Artificial sequence
  Synthetic antibody
GFDFSRDT

SEQ ID NO: 402 - protein, 8 aa, Artificial sequence
  Synthetic antibody
ISRIETYT

SEQ ID NO: 403 - protein, 17 aa, Artificial sequence
  Synthetic antibody
ARAGAVRPGIGFHYFDN

SEQ ID NO: 404 - DNA, 24 bp, Artificial sequence
  Synthetic antibody
ggattcaactttactaactactac

SEQ ID NO: 405 - DNA, 24 bp, Artificial sequence
  Synthetic antibody
attagtagtactactaatagcata

SEQ ID NO: 406 - DNA, 45 bp, Artificial sequence
  Synthetic antibody
gcgcgacatttggtcagggggacctctcttgctgcttttgatatc

SEQ ID NO: 407 - protein, 8 aa, Artificial sequence
  Synthetic antibody
GFNFTNYY

SEQ ID NO: 408 - protein, 8 aa, Artificial sequence
  Synthetic antibody
ISSTTNSI

SEQ ID NO: 409 - protein, 15 aa, Artificial sequence
  Synthetic antibody
ARHLVRGTSLAAFDI

SEQ ID NO: 410 - DNA, 24 bp, Artificial sequence
  Synthetic antibody
ggatacaccttcactaagtatggt

SEQ ID NO: 411 - DNA, 24 bp, Artificial sequence
  Synthetic antibody
attaacacgaacactgcaaagcca

SEQ ID NO: 412 - DNA, 24 bp, Artificial sequence
  Synthetic antibody
gcgacagatggtagtgagggctcc

SEQ ID NO: 413 - protein, 8 aa, Artificial sequence
  Synthetic antibody
GYTFTKYG

SEQ ID NO: 414 - protein, 8 aa, Artificial sequence
  Synthetic antibody
INTNTAKP

SEQ ID NO: 415 - protein, 8 aa, Artificial sequence
  Synthetic antibody
ATDGSEGS

SEQ ID NO: 416 - DNA, 24 bp, Artificial sequence
  Synthetic antibody
ggtgggtccttcaatggttactac

SEQ ID NO: 417 - DNA, 21 bp, Artificial sequence
  Synthetic antibody
atcgatcatagtggaagcacc

SEQ ID NO: 418 - DNA, 42 bp, Artificial sequence
  Synthetic antibody
gcgaggaggcgcattgggagcctattaaaatacttccaggac

SEQ ID NO: 419 - protein, 8 aa, Artificial sequence
  Synthetic antibody
GGSFNGYY

SEQ ID NO: 420 - protein, 7 aa, Artificial sequence
  Synthetic antibody
IDHSGST

SEQ ID NO: 421 - protein, 14 aa, Artificial sequence
  Synthetic antibody
ARRRIGSLLKYFQD

SEQ ID NO: 422 - DNA, 30 bp, Artificial sequence
  Synthetic antibody
gatgcctccatcgacactccgagttacttc

SEQ ID NO: 423 - DNA, 21 bp, Artificial sequence
  Synthetic antibody
atctattatactgggaacaag

SEQ ID NO: 424 - DNA, 42 bp, Artificial sequence
  Synthetic antibody
gcgagatatgttgattatgtttggttgagggcttttgatata

SEQ ID NO: 425 - protein, 10 aa, Artificial sequence
  Synthetic antibody
DASIDTPSYF

SEQ ID NO: 426 - protein, 7 aa, Artificial sequence
  Synthetic antibody
IYYTGNK

SEQ ID NO: 427 - protein, 14 aa, Artificial sequence
  Synthetic antibody
ARYVDYVWLRAFDI

SEQ ID NO: 428 - DNA, 24 bp, Artificial sequence
  Synthetic antibody
ggattcaccttcaatacttactgg

SEQ ID NO: 429 - DNA, 24 bp, Artificial sequence
  Synthetic antibody
ctgaataaagatggcagtagtcca

SEQ ID NO: 430 - DNA, 45 bp, Artificial sequence
  Synthetic antibody
gtcagagatcgacgagcggtgactacggcccggtacttcgatctc

SEQ ID NO: 431 - protein, 8 aa, Artificial sequence
  Synthetic antibody
GFTFNTYW

SEQ ID NO: 432 - protein, 8 aa, Artificial sequence
  Synthetic antibody
LNKDGSSP

SEQ ID NO: 433 - protein, 15 aa, Artificial sequence
  Synthetic antibody
VRDRRAVTTARYFDL

SEQ ID NO: 434 - DNA, 24 bp, Artificial sequence
  Synthetic antibody
ggatacgacctcactaaatatgct

SEQ ID NO: 435 - DNA, 24 bp, Artificial sequence
  Synthetic antibody
atcaacaccaacactgcgaaacca

SEQ ID NO: 436 - DNA, 24 bp, Artificial sequence
  Synthetic antibody
tcgagagatggtagtggggcctcc

SEQ ID NO: 437 - protein, 8 aa, Artificial sequence
  Synthetic antibody
GYDLTKYA

SEQ ID NO: 438 - protein, 8 aa, Artificial sequence
  Synthetic antibody
INTNTAKP

SEQ ID NO: 439 - protein, 8 aa, Artificial sequence
  Synthetic antibody
SRDGSGAS

SEQ ID NO: 440 - DNA, 24 bp, Artificial sequence
  Synthetic antibody
ggattcaccttcaatatgcatagc

SEQ ID NO: 441 - DNA, 24 bp, Artificial sequence
  Synthetic antibody
attagtagtactagcacttacatc

SEQ ID NO: 442 - DNA, 42 bp, Artificial sequence
  Synthetic antibody
gtgagacgaggtcggggtggagcagcccgtgcacttgacaac

SEQ ID NO: 443 - protein, 8 aa, Artificial sequence
  Synthetic antibody
GFTFNMHS

SEQ ID NO: 444 - protein, 8 aa, Artificial sequence
  Synthetic antibody
ISSTSTYI

SEQ ID NO: 445 - protein, 14 aa, Artificial sequence
  Synthetic antibody
VRRGRGGAARALDN

SEQ ID NO: 446 - DNA, 24 bp, Artificial sequence
  Synthetic antibody
ggattcattctcagtaggagtagc

SEQ ID NO: 447 - DNA, 24 bp, Artificial sequence
  Synthetic antibody
attagtagtactagtagtcatata

SEQ ID NO: 448 - DNA, 36 bp, Artificial sequence
  Synthetic antibody
gcgagaaaaaccaatggggcctggttcctcgatctc

SEQ ID NO: 449 - protein, 8 aa, Artificial sequence
  Synthetic antibody
GFILSRSS

SEQ ID NO: 450 - protein, 8 aa, Artificial sequence
  Synthetic antibody
ISSTSSHI

SEQ ID NO: 451 - protein, 12 aa, Artificial sequence
  Synthetic antibody
ARKTNGAWFLDL

SEQ ID NO: 452 - DNA, 24 bp, Artificial sequence
  Synthetic antibody
ggatacgacctcactaaatatgct

SEQ ID NO: 453 - DNA, 24 bp, Artificial sequence
  Synthetic antibody
atcaacaccaacactgcgaaacca

SEQ ID NO: 454 - DNA, 24 bp, Artificial sequence
  Synthetic antibody
tcgagagatggtagtggggcctcc

SEQ ID NO: 455 - protein, 8 aa, Artificial sequence
  Synthetic antibody
GYDLTKYA

SEQ ID NO: 456 - protein, 8 aa, Artificial sequence
  Synthetic antibody
INTNTAKP

SEQ ID NO: 457 - protein, 8 aa, Artificial sequence
  Synthetic antibody
SRDGSGAS

SEQ ID NO: 458 - DNA, 24 bp, Artificial sequence
  Synthetic antibody
ggatacaccttcactaagtatggt

SEQ ID NO: 459 - DNA, 24 bp, Artificial sequence
  Synthetic antibody
attaacacgaacactgcaaagcca

SEQ ID NO: 460 - DNA, 24 bp, Artificial sequence
  Synthetic antibody
gcgacagatggtagtgagggctcc

SEQ ID NO: 461 - protein, 8 aa, Artificial sequence
  Synthetic antibody
GYTFTKYG

SEQ ID NO: 462 - protein, 8 aa, Artificial sequence
  Synthetic antibody
INTNTAKP

SEQ ID NO: 463 - protein, 8 aa, Artificial sequence
  Synthetic antibody
ATDGSEGS

SEQ ID NO: 464 - DNA, 24 bp, Artificial sequence
  Synthetic antibody
ggatacaccttcactaactatgct

SEQ ID NO: 465 - DNA, 24 bp, Artificial sequence
  Synthetic antibody
atcaacaccaacactgggaaacca

SEQ ID NO: 466 - DNA, 42 bp, Artificial sequence
  Synthetic antibody
gcgagaggcgagtccctagcagccctcggttcgttcgcctac

SEQ ID NO: 467 - protein, 8 aa, Artificial sequence
  Synthetic antibody
GYTFTNYA

SEQ ID NO: 468 - protein, 8 aa, Artificial sequence
  Synthetic antibody
INTNTGKP

SEQ ID NO: 469 - protein, 14 aa, Artificial sequence
  Synthetic antibody
ARGESLAALGSFAY

SEQ ID NO: 470 - DNA, 24 bp, Artificial sequence
  Synthetic antibody
ttttttaactttagaatgtatccc

SEQ ID NO: 471 - DNA, 24 bp, Artificial sequence
  Synthetic antibody
attagtggaactggtcagaccaca

SEQ ID NO: 472 - DNA, 51 bp, Artificial sequence
  Synthetic antibody
gcgcaactgcatctcggttattatcatgagagcagtggttattttttaagt

SEQ ID NO: 473-477 - skipped (5 entries)

SEQ ID NO: 478 - protein, 8 aa, Artificial sequence
  Synthetic antibody
FFNFRMYP

SEQ ID NO: 479 - protein, 8 aa, Artificial sequence
  Synthetic antibody
ISGTGQTT

SEQ ID NO: 480 - protein, 17 aa, Artificial sequence
  Synthetic antibody
AQLHLGYYHESSGYFLS

SEQ ID NO: 481 - DNA, 30 bp, Artificial sequence
  Synthetic antibody
ggtggctccatcagcagtaggactttcttc

SEQ ID NO: 482 - DNA, 21 bp, Artificial sequence
  Synthetic antibody
ctcttttccggtgggaccacc

SEQ ID NO: 483 - DNA, 51 bp, Artificial sequence
  Synthetic antibody
gcgagacaacgggcagcattaccgccctactactactactacttcgacgtc

SEQ ID NO: 484 - protein, 10 aa, Artificial sequence
  Synthetic antibody
GGSISSRTFF

SEQ ID NO: 485 - protein, 7 aa, Artificial sequence
  Synthetic antibody
LFSGGTT

SEQ ID NO: 486 - protein, 17 aa, Artificial sequence
  Synthetic antibody
ARQRAALPPYYYYYFDV

SEQ ID NO: 487 - DNA, 24 bp, Artificial sequence
  Synthetic antibody
ggattcaggttcaatgagcatagt

SEQ ID NO: 488 - DNA, 24 bp, Artificial sequence
  Synthetic antibody
attactagtagcggaaataccata

SEQ ID NO: 489 - DNA, 48 bp, Artificial sequence
  Synthetic antibody
gcgagagatgggggttgtaatggtcgcacctgctacggactcaactac

SEQ ID NO: 490 - protein, 8 aa, Artificial sequence
  Synthetic antibody
GFRFNEHS

SEQ ID NO: 491 - protein, 8 aa, Artificial sequence
  Synthetic antibody
ITSSGNTI

SEQ ID NO: 492 - protein, 16 aa, Artificial sequence
  Synthetic antibody
ARDGGCNGRTCYGLNY

SEQ ID NO: 493 - DNA, 24 bp, Artificial sequence
  Synthetic antibody
ggattcaccttcagtagttatggc

SEQ ID NO: 494 - DNA, 24 bp, Artificial sequence
  Synthetic antibody
atacggtataatggaagtaataaa

SEQ ID NO: 495 - DNA, 54 bp, Artificial sequence
  Synthetic antibody
gcgaaagacggcgattacgattcttggagtggtctcactgaacacttccagcac

SEQ ID NO: 496 - protein, 8 aa, Artificial sequence
  Synthetic antibody
GFTFSSYG

SEQ ID NO: 497 - protein, 8 aa, Artificial sequence
  Synthetic antibody
IRYNGSNK

SEQ ID NO: 498 - protein, 18 aa, Artificial sequence
  Synthetic antibody
AKDGDYDSWSGLTEHFQH

SEQ ID NO: 499 - DNA, 24 bp, Artificial sequence
  Synthetic antibody
ggattcgacttccctggctatggc

SEQ ID NO: 500 - DNA, 24 bp, Artificial sequence
  Synthetic antibody
atatggtacgatgccagaagtgaa

SEQ ID NO: 501 - DNA, 27 bp, Artificial sequence
  Synthetic antibody
gcggtgggagctcctctcgaagggtac

SEQ ID NO: 502 - protein, 8 aa, Artificial sequence
  Synthetic antibody
GFDFPGYG

SEQ ID NO: 503 - protein, 8 aa, Artificial sequence
  Synthetic antibody
IWYDARSE

SEQ ID NO: 504 - protein, 9 aa, Artificial sequence
  Synthetic antibody
AVGAPLEGY

SEQ ID NO: 505 - DNA, 24 bp, Artificial sequence
  Synthetic antibody
ggtggctccatcagtagttactac

SEQ ID NO: 506 - DNA, 21 bp, Artificial sequence
  Synthetic antibody
atctttaccactgggagcacc

SEQ ID NO: 507 - DNA, 48 bp, Artificial sequence
  Synthetic antibody
gcgagactgaggagggtagtaccaactgctatctggcacttcgatctc

SEQ ID NO: 508 - protein, 8 aa, Artificial sequence
  Synthetic antibody
GGSISSYY

SEQ ID NO: 509 - protein, 7 aa, Artificial sequence
  Synthetic antibody
IFTTGST

SEQ ID NO: 510 - protein, 16 aa, Artificial sequence
  Synthetic antibody
ARLRRVVPTAIWHFDL

SEQ ID NO: 511 - DNA, 36 bp, Artificial sequence
  Synthetic antibody
cagagtgttttagacagctccaacaataagaagtac

SEQ ID NO: 512 - DNA, 27 bp, Artificial sequence
  Synthetic antibody
cagcaatattatagtactcctccgacg

SEQ ID NO: 513 - protein, 12 aa, Artificial sequence
  Synthetic antibody
QSVLDSSNNKKY

SEQ ID NO: 514 - protein, 9 aa, Artificial sequence
  Synthetic antibody
QQYYSTPPT

SEQ ID NO: 515 - DNA, 18 bp, Artificial sequence
  Synthetic antibody
gagaatatttataagtgg

SEQ ID NO: 516 - DNA, 27 bp, Artificial sequence
  Synthetic antibody
caacattataacagtttccccttcact

SEQ ID NO: 517 - protein, 6 aa, Artificial sequence
  Synthetic antibody
ENIYKW

SEQ ID NO: 518 - protein, 9 aa, Artificial sequence
  Synthetic antibody
QHYNSFPFT

SEQ ID NO: 519 - DNA, 36 bp, Artificial sequence; identical to SEQ ID NO: 511
  Synthetic antibody

SEQ ID NO: 520 - DNA, 27 bp, Artificial sequence
  Synthetic antibody
cagcaatattatactactcctccgacg

SEQ ID NO: 521 - protein, 12 aa, Artificial sequence
  Synthetic antibody
QSVLDSSNNKKY

SEQ ID NO: 522 - protein, 9 aa, Artificial sequence
  Synthetic antibody
QQYYTTPPT

SEQ ID NO: 523 - DNA, 18 bp, Artificial sequence
  Synthetic antibody
aaattgggggccaaattt

SEQ ID NO: 524 - DNA, 27 bp, Artificial sequence
  Synthetic antibody
caggcgtgggaaagtggtattgtggcg

SEQ ID NO: 525 - protein, 6 aa, Artificial sequence
  Synthetic antibody
KLGAKF

SEQ ID NO: 526 - protein, 9 aa, Artificial sequence
  Synthetic antibody
QAWESGIVA

SEQ ID NO: 527 - DNA, 18 bp, Artificial sequence
  Synthetic antibody
caggacattggacactat

SEQ ID NO: 528 - DNA, 30 bp, Artificial sequence
  Synthetic antibody
catcagcatgagaagctttactcgatctcc

SEQ ID NO: 529 - protein, 6 aa, Artificial sequence
  Synthetic antibody
QDIGHY

SEQ ID NO: 530 - protein, 10 aa, Artificial sequence
  Synthetic antibody
HQHEKLYSIS

SEQ ID NO: 531 - DNA, 18 bp, Artificial sequence
  Synthetic antibody
aacattgcaacaaaacgt

SEQ ID NO: 532 - DNA, 33 bp, Artificial sequence
  Synthetic antibody
caggtgtgggatagtctggctgaccaagtggta

SEQ ID NO: 533 - protein, 6 aa, Artificial sequence
  Synthetic antibody
NIATKR

SEQ ID NO: 534 - protein, 11 aa, Artificial sequence
  Synthetic antibody
QVWDSLADQVV

SEQ ID NO: 535 - DNA, 18 bp, Artificial sequence
  Synthetic antibody
cagagtattagccggtgg

SEQ ID NO: 536 - DNA, 27 bp, Artificial sequence
  Synthetic antibody
caagaatatactaattattcggggacg

SEQ ID NO: 537 - protein, 6 aa, Artificial sequence
  Synthetic antibody
QSISRW

SEQ ID NO: 538 - protein, 9 aa, Artificial sequence
  Synthetic antibody
QEYTNYSGT

SEQ ID NO: 539 - DNA, 18 bp, Artificial sequence
  Synthetic antibody
acattgggtgatagatat

SEQ ID NO: 540 - DNA, 27 bp, Artificial sequence
  Synthetic antibody
ctgacgtgggaccgcggcactcctgtc

SEQ ID NO: 541 - protein, 6 aa, Artificial sequence
  Synthetic antibody
TLGDRY

SEQ ID NO: 542 - protein, 9 aa, Artificial sequence
  Synthetic antibody
LTWDRGTPV

SEQ ID NO: 543 - DNA, 18 bp, Artificial sequence
  Synthetic antibody
ggaattggcaggaaacgt

SEQ ID NO: 544 - DNA, 33 bp, Artificial sequence
  Synthetic antibody
caggtgtgggatagtaggactgaccaagtggtg

SEQ ID NO: 545 - protein, 6 aa, Artificial sequence
  Synthetic antibody
GIGRKR

SEQ ID NO: 546 - protein, 11 aa, Artificial sequence
  Synthetic antibody
QVWDSRTDQVV

SEQ ID NO: 547 - DNA, 18 bp, Artificial sequence
  Synthetic antibody
cagagtgttgacacctac

SEQ ID NO: 548 - DNA, 30 bp, Artificial sequence
  Synthetic antibody
cagcagcgtaacaactggcggacgtggacg

SEQ ID NO: 549 - protein, 6 aa, Artificial sequence
  Synthetic antibody
QSVDTY

SEQ ID NO: 550 - protein, 10 aa, Artificial sequence
  Synthetic antibody
QQRNNWRTWT

SEQ ID NO: 551 - DNA, 18 bp, Artificial sequence
  Synthetic antibody
aacattgccaagaaacgt

SEQ ID NO: 552 - DNA, 33 bp, Artificial sequence
  Synthetic antibody
caggtgtgggatagtaagactgaccacgtggtt

SEQ ID NO: 553 - protein, 6 aa, Artificial sequence
  Synthetic antibody
NIAKKR

SEQ ID NO: 554 - protein, 11 aa, Artificial sequence
  Synthetic antibody
QVWDSKTDHVV

SEQ ID NO: 555 - DNA, 33 bp, Artificial sequence
  Synthetic antibody
cagagcctcctccataggaatggatacaactat

SEQ ID NO: 556 - DNA, 24 bp, Artificial sequence
  Synthetic antibody
atgcaagctctacaaacttggacg

SEQ ID NO: 557 - protein, 11 aa, Artificial sequence
  Synthetic antibody
QSLLHRNGYNY

SEQ ID NO: 558 - protein, 8 aa, Artificial sequence
  Synthetic antibody
MQALQTWT

SEQ ID NO: 559 - DNA, 18 bp, Artificial sequence
  Synthetic antibody
aacattgtaagaaaacgt

SEQ ID NO: 560 - DNA, 33 bp, Artificial sequence
  Synthetic antibody
caggtgtgggatagtacgactgaccaggtggta

SEQ ID NO: 561 - protein, 6 aa, Artificial sequence
  Synthetic antibody
NIVRKR

SEQ ID NO: 562 - protein, 11 aa, Artificial sequence
  Synthetic antibody
QVWDSTTDQVV

SEQ ID NO: 563 - DNA, 36 bp, Artificial sequence
  Synthetic antibody
cagaatattttagacaactccaacaataagaacttc

SEQ ID NO: 564 - DNA, 27 bp, Artificial sequence
  Synthetic antibody
ctccaatattatagtcttcctcacact

SEQ ID NO: 565 - protein, 12 aa, Artificial sequence
  Synthetic antibody
QNILDNSNNKNF

SEQ ID NO: 566 - protein, 9 aa, Artificial sequence
  Synthetic antibody
LQYYSLPHT

SEQ ID NO: 567 - DNA, 18 bp, Artificial sequence
  Synthetic antibody
aaattgggtgaaagatat

SEQ ID NO: 568 - DNA, 27 bp, Artificial sequence
  Synthetic antibody
ctgacgtgggaccgcggcactcctgtc

SEQ ID NO: 569 - protein, 6 aa, Artificial sequence
  Synthetic antibody
KLGERY

SEQ ID NO: 570 - protein, 9 aa, Artificial sequence
  Synthetic antibody
LTWDRGTPV

SEQ ID NO: 571 - DNA, 18 bp, Artificial sequence
  Synthetic antibody
cagaccattagtacttat

SEQ ID NO: 572 - DNA, 27 bp, Artificial sequence
  Synthetic antibody
caacagggttacaataacccgtacact

SEQ ID NO: 573 - protein, 6 aa, Artificial sequence
  Synthetic antibody
QTISTY

SEQ ID NO: 574 - protein, 9 aa, Artificial sequence
  Synthetic antibody
QQGYNNPYT

SEQ ID NO: 575 - DNA, 33 bp, Artificial sequence
  Synthetic antibody
cagagcctcctggatcgtaatggatacaactac

SEQ ID NO: 576 - DNA, 27 bp, Artificial sequence
  Synthetic antibody
atgcaagctctacaaattccgatcacc

SEQ ID NO: 577 - protein, 11 aa, Artificial sequence
  Synthetic antibody
QSLLDRNGYNY

SEQ ID NO: 578 - protein, 9 aa, Artificial sequence
  Synthetic antibody
MQALQIPIT

SEQ ID NO: 579 - DNA, 33 bp, Artificial sequence; identical to SEQ ID NO: 575
  Synthetic antibody

SEQ ID NO: 580 - DNA, 27 bp, Artificial sequence
  Synthetic antibody
atgcaagctctacaaattccgatcacc

SEQ ID NO: 581 - protein, 11 aa, Artificial sequence
  Synthetic antibody
QSLLDRNGYNY

SEQ ID NO: 582 - protein, 9 aa, Artificial sequence
  Synthetic antibody
MQALQIPIT

SEQ ID NO: 583 - DNA, 27 bp, Artificial sequence
  Synthetic antibody
aatagtgacattcgtgattatgattat

SEQ ID NO: 584 - DNA, 33 bp, Artificial sequence
  Synthetic antibody
tgttcgtattcagtcacttactctttcgaggtc

SEQ ID NO: 585 - protein, 9 aa, Artificial sequence
  Synthetic antibody
NSDIRDYDY

SEQ ID NO: 586 - protein, 11 aa, Artificial sequence
  Synthetic antibody
CSYSVTYSFEV

SEQ ID NO: 587 - DNA, 33 bp, Artificial sequence; identical to SEQ ID NO: 575
  Synthetic antibody

SEQ ID NO: 588 - DNA, 27 bp, Artificial sequence
  Synthetic antibody
atgcaagctctacaaattccgatcacc

SEQ ID NO: 589 - protein, 11 aa, Artificial sequence
  Synthetic antibody
QSLLDRNGYNY

SEQ ID NO: 590 - protein, 9 aa, Artificial sequence
  Synthetic antibody
MQALQIPIT

SEQ ID NO: 591 - DNA, 18 bp, Artificial sequence
  Synthetic antibody
aacttgggggataagtat

SEQ ID NO: 592 - DNA, 30 bp, Artificial sequence
  Synthetic antibody
caggcgttggacagtggcagtttttgggtg

SEQ ID NO: 593 - protein, 6 aa, Artificial sequence
  Synthetic antibody
NLGDKY

SEQ ID NO: 594 - protein, 10 aa, Artificial sequence
  Synthetic antibody
QALDSGSFWV

SEQ ID NO: 595 - DNA, 33 bp, Artificial sequence; identical to SEQ ID NO: 575
  Synthetic antibody

SEQ ID NO: 596 - DNA, 27 bp, Artificial sequence
  Synthetic antibody
atgcaagctctacaaattccgatcacc

SEQ ID NO: 597 - protein, 11 aa, Artificial sequence
  Synthetic antibody
QSLLDRNGYNY

SEQ ID NO: 598 - protein, 9 aa, Artificial sequence
  Synthetic antibody
MQALQIPIT

SEQ ID NO: 599 - DNA, 18 bp, Artificial sequence
  Synthetic antibody
ccgagtgttggcaggttc

SEQ ID NO: 600 - DNA, 27 bp, Artificial sequence
  Synthetic antibody
cagcaccgtagcaactggccggtcact

SEQ ID NO: 601 - protein, 6 aa, Artificial sequence
  Synthetic antibody
PSVGRF

SEQ ID NO: 602 - protein, 9 aa, Artificial sequence
  Synthetic antibody
QHRSNWPVT

SEQ ID NO: 603 - DNA, 18 bp, Artificial sequence
  Synthetic antibody
cagagcattggtcgctgg

SEQ ID NO: 604 - DNA, 27 bp, Artificial sequence
  Synthetic antibody
caacaatataatactaattcggggaca

SEQ ID NO: 605 - protein, 6 aa, Artificial sequence
  Synthetic antibody
QSIGRW

SEQ ID NO: 606 - protein, 9 aa, Artificial sequence
  Synthetic antibody
QQYNTNSGT

SEQ ID NO: 607 - DNA, 18 bp, Artificial sequence
  Synthetic antibody
cagagtattggtacctgg

SEQ ID NO: 608 - DNA, 27 bp, Artificial sequence
  Synthetic antibody
caacaatataatacttactcggggacg

SEQ ID NO: 609 - protein, 6 aa, Artificial sequence
  Synthetic antibody
QSIGTW

SEQ ID NO: 610 - protein, 9 aa, Artificial sequence
  Synthetic antibody
QQYNTYSGT

SEQ ID NO: 611 - DNA, 18 bp, Artificial sequence
  Synthetic antibody
aaattgggggatagttat

SEQ ID NO: 612 - DNA, 30 bp, Artificial sequence
  Synthetic antibody
caggcgtgggacaccaccacggattgggtg

SEQ ID NO: 613 - protein, 6 aa, Artificial sequence
  Synthetic antibody
KLGDSY

SEQ ID NO: 614 - protein, 10 aa, Artificial sequence
  Synthetic antibody
QAWDTTTDWV

SEQ ID NO: 615 - DNA, 18 bp, Artificial sequence
  Synthetic antibody
ccgagtgctggccgcttc

SEQ ID NO: 616 - DNA, 27 bp, Artificial sequence
  Synthetic antibody
caacaccgtagcaactggccgctcact

SEQ ID NO: 617 - protein, 6 aa, Artificial sequence
  Synthetic antibody
PSAGRF

SEQ ID NO: 618 - protein, 9 aa, Artificial sequence
  Synthetic antibody
QHRSNWPLT

SEQ ID NO: 619 - DNA, 18 bp, Artificial sequence
  Synthetic antibody
tccaacattgataactac

SEQ ID NO: 620 - DNA, 30 bp, Artificial sequence
  Synthetic antibody
caacaatattatactagtcctccggtcact

SEQ ID NO: 621 - protein, 6 aa, Artificial sequence
  Synthetic antibody
SNIDNY

SEQ ID NO: 622 - protein, 10 aa, Artificial sequence
  Synthetic antibody
QQYYTSPPVT

SEQ ID NO: 623 - DNA, 18 bp, Artificial sequence
  Synthetic antibody
cagaatattggtcgctgg

SEQ ID NO: 624 - DNA, 27 bp, Artificial sequence
  Synthetic antibody
cagcaatatactacttattcggggacg

SEQ ID NO: 625 - protein, 6 aa, Artificial sequence
  Synthetic antibody
QNIGRW

SEQ ID NO: 626 - protein, 9 aa, Artificial sequence
  Synthetic antibody
QQYTTYSGT

SEQ ID NO: 627 - DNA, 18 bp, Artificial sequence
  Synthetic antibody
aacactggaagtaaaagt

SEQ ID NO: 628 - DNA, 33 bp, Artificial sequence
  Synthetic antibody
caggtgtgggatagtactattgatcatgtgata

SEQ ID NO: 629 - protein, 6 aa, Artificial sequence
  Synthetic antibody
NTGSKS

SEQ ID NO: 630 - protein, 11 aa, Artificial sequence
  Synthetic antibody
QVWDSTIDHVI

SEQ ID NO: 631 - DNA, 27 bp, Artificial sequence
  Synthetic antibody
agctccaacatcggggcaggttctgat

SEQ ID NO: 632 - DNA, 36 bp, Artificial sequence
  Synthetic antibody
cagtcctatgactacgacctgagtgggtcttgggtc

SEQ ID NO: 633 - protein, 9 aa, Artificial sequence
  Synthetic antibody
SSNIGAGSD

SEQ ID NO: 634 - protein, 12 aa, Artificial sequence
  Synthetic antibody
QSYDYDLSGSWV

SEQ ID NO: 635 - DNA, 18 bp, Artificial sequence
  Synthetic antibody
cagagtattggtacctgg

SEQ ID NO: 636 - DNA, 27 bp, Artificial sequence
  Synthetic antibody
caacaatataatacttactcggggacg

SEQ ID NO: 637 - protein, 6 aa, Artificial sequence
  Synthetic antibody
QSIGTW

SEQ ID NO: 638 - protein, 9 aa, Artificial sequence
  Synthetic antibody
QQYNTYSGT

SEQ ID NO: 639 - DNA, 18 bp, Artificial sequence
  Synthetic antibody
cagagcattaacagctat

SEQ ID NO: 640 - DNA, 27 bp, Artificial sequence
  Synthetic antibody
caacagagttccagtactttttggacg

SEQ ID NO: 641 - protein, 6 aa, Artificial sequence
  Synthetic antibody
QSINSY

SEQ ID NO: 642 - protein, 9 aa, Artificial sequence
  Synthetic antibody
QQSSSTFWT

SEQ ID NO: 643 - DNA, 18 bp, Artificial sequence
  Synthetic antibody
cagactataagtagttgg

SEQ ID NO: 644 - DNA, 24 bp, Artificial sequence
  Synthetic antibody
caacagtatactagttcttggacg

SEQ ID NO: 645 - protein, 6 aa, Artificial sequence
  Synthetic antibody
QTISSW

SEQ ID NO: 646 - protein, 8 aa, Artificial sequence
  Synthetic antibody
QQYTSSWT

SEQ ID NO: 647 - DNA, 18 bp, Artificial sequence
  Synthetic antibody
cagagtgttggcagatac

SEQ ID NO: 648 - DNA, 30 bp, Artificial sequence
  Synthetic antibody
cagcagcgtggcaactggcctccgatcacc

SEQ ID NO: 649 - protein, 6 aa, Artificial sequence
  Synthetic antibody
QSVGRY

SEQ ID NO: 650 - protein, 10 aa, Artificial sequence
  Synthetic antibody
QQRGNWPPIT

SEQ ID NO: 651 - DNA, 24 bp, Artificial sequence
  Synthetic antibody
agatccgacattgggaatagtttt

SEQ ID NO: 652 - DNA, 33 bp, Artificial sequence
  Synthetic antibody
ggaacatgggatatcagcctgagtgcggcggtc

SEQ ID NO: 653 - protein, 8 aa, Artificial sequence
  Synthetic antibody
RSDIGNSF

SEQ ID NO: 654 - protein, 11 aa, Artificial sequence
  Synthetic antibody
GTWDISLSAAV

SEQ ID NO: 655 - DNA, 36 bp, Artificial sequence
  Synthetic antibody
cagaatgttttatacagctccaacaataagaactac

SEQ ID NO: 656 - DNA, 27 bp, Artificial sequence
  Synthetic antibody
cagcaatattatagtatacctccggct

SEQ ID NO: 657 - protein, 12 aa, Artificial sequence
  Synthetic antibody
QNVLYSSNNKNY

SEQ ID NO: 658 - protein, 9 aa, Artificial sequence
  Synthetic antibody
QQYYSIPPA

SEQ ID NO: 659 - DNA, 18 bp, Artificial sequence
  Synthetic antibody
cagaacattgccgactat

SEQ ID NO: 660 - DNA, 30 bp, Artificial sequence
  Synthetic antibody
cagcagagttacacttcacccacgtggacg

SEQ ID NO: 661 - protein, 6 aa, Artificial sequence
  Synthetic antibody
QNIADY

SEQ ID NO: 662 - protein, 10 aa, Artificial sequence
  Synthetic antibody
QQSYTSPTWT

SEQ ID NO: 663 - DNA, 27 bp, Artificial sequence
  Synthetic antibody
tctggcgcagtctctactagttacttc

SEQ ID NO: 664 - DNA, 30 bp, Artificial sequence
  Synthetic antibody
gtgttgtatatggatagtggcgtgatggta

SEQ ID NO: 665 - protein, 9 aa, Artificial sequence
  Synthetic antibody
SGAVSTSYF

SEQ ID NO: 666 - protein, 10 aa, Artificial sequence
  Synthetic antibody
VLYMDSGVMV